(19) United States
(12) Patent Application Publication
(10) Pub. No.: US 2011/0047645 A1
(43) Pub. Date: Feb. 24, 2011

(54) Compositions and Methods of Using RNA Interference for Control of Nematodes

(75) Inventors: Shawn Motyka, Apex, NC (US); Bonnie McCaig, Durham, NC (US); Peifeng Ron, Cary, NC (US); John McMillan, Raleigh, NC (US); Lawrence Talton, Cary, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/989,961

(22) PCT Filed: Apr. 29, 2009
(86) PCT No.: PCT/EP2009/055170

§ 371 Date: Oct. 28, 2010

Related U.S. Application Data
(60) Provisional application No. 61/049,001, filed on Apr. 30, 2008.

(51) Int. Cl. A01H 1/06; C07H 21/02; C40B 40/08; A01H 5/00; C12N 15/63

(52) U.S. Cl. 800/279; 536/23.2; 536/23.5; 506/17; 800/295; 435/320.1

(57) ABSTRACT

The present invention provides double stranded RNA compositions and transgenic plants capable of inhibiting expression of essential genes in parasitic nematodes, and methods associated therewith. Specifically, the invention relates to the use of RNA interference to inhibit expression of a target essential nematode gene, which is a nematode innexin-like, pas-1, tep-1, snurportin-1 like, pol delta S, prs-4, rtp-1 or rpn-5 gene, and relates to the generation of plants that have increased resistance to parasitic nematodes,

The field of this invention is the control of nematodes, in particular the control of soybean cyst nematodes. The invention also relates to the introduction of genetic material into plants that are susceptible to nematodes in order to increase resistance to nematodes.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematode infestation can cause significant yield losses without any obvious above-ground disease symptoms. The primary causes of yield reduction are due to root damage underground. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs were also effective at inducing RNAi in C. elegans. It is known that when hairpin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into 19-24 nucleotide fragments (siRNA) within cells, and that these siRNAs are the actual mediators of the RNAi phenomenon.

In plants, long dsRNA is processed into siRNA duplexes of 21 nucleotides by an RNAse III designated as “Dicer”. The 21-nucleotide siRNA duplex in plants may comprise a 19-nucleotide double stranded portion and a 2-nucleotide overhanging portion at the 3′ end of each RNA strand. It has been shown that the 2-nucleotide overhanging portions do not contribute to sequence-specific gene silencing, and that the 19-nucleotide double stranded portion actually mediates sequence-specific gene silencing. (Elbashir (2001) Nature 411:494-498).

Use of RNAi to target essential nematode genes has been proposed, for example, in PCT Publication WO 01/96584, WO 01/17654, US 2004/0098761, US 2005/0091713, US 2005/0188438, US 2006/0037101, US 2006/0080749, US 2007/0199100, and US 2007/0250947. Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present invention provides nucleic acids, transgenic plants, and methods to overcome or alleviate nematode infestation of valuable agricultural crops such as soybeans. The nucleic acids of the invention are capable of decreasing expression of parasitic nematode target genes by RNAi. In accordance with the invention, the parasitic nematode target gene is selected from a group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to the C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

The nucleic acids of the invention encode double stranded RNA comprising (a) a first strand having a sequence substantially identical to from 19 to about 400 or 500 consecutive nucleotides of a target gene having a sequence selected from the group of SEQ ID NO:1, SEQ ID NO:5, SEQ ID NO:11; SEQ ID NO:19, SEQ ID NO:23, SEQ ID NO:104, SEQ ID NO:39 and SEQ ID NO:57 and (b) a second strand having a sequence substantially complementary to the first strand.

The invention is further embodied as a pool of double stranded RNA molecules comprising a multiplicity of short interfering RNA molecules each comprising a double stranded region having a length of 19 to 24 nucleotides, wherein said RNA molecules are derived from a polynucleotide selected from the group consisting of (a) a polynucleotide having a sequence as set forth in SEQ ID NO:1; (b) a polynucleotide having a sequence as set forth in SEQ ID NO:5; (c) a polynucleotide having a sequence as set forth in SEQ ID NO:11; (d) a polynucleotide having a sequence as set forth in SEQ ID NO:19; (e) a polynucleotide having a sequence as set forth in SEQ ID NO:23; (f) a polynucleotide having a sequence as set forth in SEQ ID NO:104; (g) a polynucleotide having a sequence as set forth in SEQ ID NO:39; (h) a polynucleotide comprising a sequence as set forth in SEQ ID NO:57.

In another embodiment, the invention provides a transgenic plant resistant to parasitic nematode infection, the plant comprising a nucleic acid construct that encodes a dsRNA or siRNA capable of specifically decreasing a parasitic nematode target gene selected from the group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

In another embodiment, the invention provides a transgenic plant capable of expressing a pool of dsRNA molecules, wherein each dsRNA molecule comprises a double stranded region having a length of 19-24 nucleotides and wherein the RNA molecules are derived from polynucleotides substantially identical to a portion of a parasitic nematode target gene selected from the group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

The invention further encompasses a method of making a transgenic plant capable of expressing a dsRNA or siRNA that is substantially identical to portion of a target gene of a parasitic nematode, said method comprising the steps of: (a) selecting a target gene from the group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene; (b) preparing a nucleic acid sequence comprising a region that is substantially identical to a portion of the selected target gene, wherein the nucleic acid is able to form a double-stranded transcript once expressed in the plant; (c) transforming a recipient plant with said nucleic acid; (d) producing one or more transgenic offspring of said recipient plant; and (e) selecting the offspring for nematode resistance.

The invention further provides a method of conferring nematode resistance to a plant, said method comprising the steps of: (a) selecting a target gene from the group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene; (b) preparing a nucleic acid sequence comprising a region that is substantially identical to a portion of the selected target gene, wherein the nucleic acid is able to form a double-stranded RNA once expressed in the plant; (c) transforming a recipient plant with said nucleic acid; (d) producing one or more transgenic offspring of said recipient plant; and (e) selecting the offspring for nematode resistance.

The invention further provides an expression cassette and an expression vector comprising a sequence substantially identical to a portion of a plant parasitic nematode target gene selected from a group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

### BRIEF DESCRIPTION OF THE DRAWINGS

FIGS. 1a-1c show the table of SEQ ID NOs assigned to corresponding nucleotide and amino acid sequences from H. glycines and other nematode species. SEQ ID NOs 1, 5, 11, 19, 23, 104, 39 and 57 correspond to full length H. glycines nucleotide sequences for innexin-like (inx, SEQ ID NO:1), pas-1 (SEQ ID NO:5), T-complex protein 1 (tcp-1, SEQ ID NO:11), snurportin 1 (SEQ ID NO:19), polymerase delta small subunit (Pol DeltaS, SEQ ID NO:23), proteasome regulatory subunit 4 (prs-4, SEQ ID NO: 104), proteasome regulatory particle, ATPase-like (rpt-1, SEQ ID NO:39) and non-ATPase proteasome regulatory subunit 5 (rpn-5, SEQ ID NO: 57) genes. The sense nucleotide fragments synthesized into hairpin expression constructs, as described in Example 2, are indicated by SEQ ID NO:3 (innexin-like), SEQ ID NO:7 (pas-1), SEQ ID NO:13 (tcp-1), SEQ ID NO: 21 (snurportin 1), SEQ ID NO:25 (Pol DeltaS), SEQ ID NO:29 (proteasome regulatory subunit 4, prs-4), SEQ ID NO:41 (rpt-1) and SEQ ID NO:59 (rpn-5). Syncytia-induced promoter sequences are given in SEQ ID NO:69 (TPP-like promoter from Arabidopsis thaliana), SEQ ID NO:70 (MtN3-like promoter from Glycine max) and SEQ ID NO:71 (promoter from locus At5g12170 from A. thaliana). Conserved nucleotide motifs are listed for pas-1 (SEQ ID NOs 72-78), rpn-5 (SEQ ID NOs 79-85), tcp-1 (SEQ ID NOs 86-91), prs-4 (SEQ ID NOs 92, 93, 106, and 107), and rpt-1 (SEQ ID NOs 94-103).

FIG. 2 shows the amino acid alignment of pas-1 like sequences: full length H. glycines pas-1 (SEQ ID NO:6); the H. glycines pas-1 fragment (SEQ ID NO:8) targeted by binary vector RTP1095; and a Globodera rostochiensis partial-length expressed sequence tag (EST) from Genbank accession number BM355389 (SEQ ID NO:10) using the Vector NTI software suite v10.3.0 (gap opening penalty=10, gap extension penalty=0.05, gap separation penalty=8).

FIG. 3 shows the amino acid alignment of tcp-1 like sequences from C. elegans Genbank accession AAA93233 (SEQ ID NO:18); the full length H. glycines tcp-1 (SEQ ID NO:12); the H. glycines tcp-1 fragment targeted by binary vector RSA131 (SEQ ID NO:14); a Heterodera schachtii partial-length expressed sequence tag (EST) from Genbank accession number CF100567 (SEQ ID NO:16), using the Vector NTI software suite v10.3.0 (gap opening penalty=10, gap extension penalty=0.05, gap separation penalty=8).

FIG. 4 shows the amino acid alignment of prs-4 like sequences from C. elegans Genbank accession 016368 (SEQ ID NO:34); C. briggsae EMBL accession CAE64528 (SEQ ID NO:32) the full length H. glycines prs-4 generated via 5′ RACE PCR (SEQ ID NO:105); the synthesized H. glycines prs-4 fragment targeted by binary vector RTP1169 (SEQ ID NO:30); the partial Contig526 assembled from Meloidogyne hapla ESTs (SEQ ID NO:36): and the partial Contig2153 assembled from Meloidogyne incognita ESTs (SEQ ID NO:38), using the Vector NTI software suite v10.3.0 (gap opening penalty=10, gap extension penalty=0.05, gap separation penalty=8).

FIGS. 5a-5b show the amino acid alignment of rpt-1 like sequences from C. elegans EMBL accession CAB01414 (SEQ ID NO:54); C. briggsae EMBL accession CAE75362 (SEQ ID NO:56); the full length H. glycines rpt-1 (SEQ ID NO:40); the H. glycines EST sequence from Genbank accession CB376265 (SEQ ID NO:44); the H. glycines rpt-1 fragment targeted by binary vector RSA012 (SEQ ID NO:42); a H. schachtii EST from Genbank accession CD750393 (SEQ ID NO:46); a G. rostochiensis EST from Genbank accession EE269079 (SEQ ID NO:50); a G. rostochiensis EST from Genbank accession EE269080 (SEQ ID NO:48); and a partial Contig1170 from Meloidogyne hapla ESTs (SEQ ID NO:52), using the Vector NTI software suite v10.3.0 (gap opening penalty=10, gap extension penalty=0.05, gap separation penalty=8).

FIGS. 6a-6b show the amino acid alignment of rpn-5 like proteins from C. elegans Genbank accession AAA81126 (SEQ ID NO:66); C. briggsae EMBL accession CAE60648 (SEQ ID NO:68); the full length H. glycines rpn-5 (SEQ ID NO:58); the H. glycines EST from Genbank accession CA940612 (SEQ ID NO:62); the partial H. glycines EST from Genbank accession CA940612 (SEQ ID NO:62); the H. glycines rpn-5 fragment targeted by binary vector RTP1269 (SEQ ID NO:60;) and a G. rostochiensis EST from Genbank accession EE266903 (SEQ ID NO:64), using the Vector NTI software suite v10.3.0 (gap opening penalty=10, gap extension penalty=0.05, gap separation penalty=8).

FIGS. 7a-7b show the nucleotide alignment of the full length H. glycines pas-1 coding region (SEQ ID NO:5), the synthesized H. glycines pas-1 fragment (SEQ ID NO:7) used in binary vector RTP1095-1 and the partial G. rostochiensis BM355389 EST (SEQ ID NO:9). Conserved motifs are indicated by bold text and are listed in FIG. 12. The alignment was done using the Vector NTI software suite v10.3.0 (gap opening penalty=15, gap extension penalty=6.66, gap separation penalty=8).

FIGS. 8a-8c show the nucleotide alignment of the full length H. glycines tcp-1 coding region (SEQ ID NO:11) and the partial H. schachtii CF100567 EST (SEQ ID NO:15). Conserved motifs are indicated by bold text and are listed in FIG. 12. The alignment was done using the Vector NTI software suite v10.3.0 (gap opening penalty=15, gap extension penalty=6.66, gap separation penalty=8).

FIGS. 9a-9b show the nucleotide alignment of the full length H. glycines prs-4 coding region (SEQ ID NO:104), the partial EST assembly for M. hapla Contig526 (SEQ ID NO:35) and the full length EST assembly for M. incognita Contig2153 (SEQ ID NO:37). Conserved motifs are indicated by bold text and are listed in FIG. 12. The alignment was done using the Vector NTI software suite v10.3.0 (gap opening penalty=15, gap extension penalty=6.66, gap separation penalty=8).

FIGS. 10a-10e show the nucleotide alignment of the full length H. glycines rpt-1 coding region (SEQ ID NO:39), the partial H. glycines CB376265 EST (SEQ ID NO:43), the partial H. schachtii CD750393 EST (SEQ ID NO:45), the partial G. rostochiensis EE269079 EST (SEQ ID NO:49), the partial G. rostochiensis EE269080 EST (SEQ ID NO:47) and the partial EST assembly for M. hapla Contig 1170 (SEQ ID NO:51). Conserved motifs are indicated by bold text and are listed in FIG. 12. The alignment was done using the Vector NTI software suite v10.3.0 (gap opening penalty=15, gap extension penalty=6.66, gap separation penalty=8).

FIGS. 11a-11b show the nucleotide alignment of the full length H. glycines rpn-5 coding region (SEQ ID NO:57) and the partial G. rostochiensis EST EE266903 (SEQ ID NO:63). Conserved motifs are indicated by bold text and are listed in FIG. 12. The alignment was done using the Vector NTI software suite v10.3.0 (gap opening penalty=15, gap extension penalty=6.66, gap separation penalty=8).

FIG. 12 shows a table of conserved nucleotide motifs identified from pas-1, rpn-5, tcp-1, prs-4 and rpt-1 genes as described in FIGS. 7-11.

FIGS. 13a-13j show global percent identity of exemplary pas-1 like sequences (FIG. 13a, amino acid; FIG. 13b, nucleotide), tcp-1 like sequences (FIG. 13c, amino acid; FIG. 13d, nucleotide), prs-4 like sequences (FIG. 13e, amino acid; FIG. 13f, nucleotide), rpt-1 like sequences (FIG. 13g, amino acid; FIG. 13h, nucleotide) and rpn-5 like sequences (FIG. 13i, amino acid; FIG. 13j, nucleotide). Percent identity was calculated from multiple alignments using the Vector NTI software suite v10.3.0.

FIGS. 14a-14l show various 2lmers possible in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67 or 104 by nucleotide position.

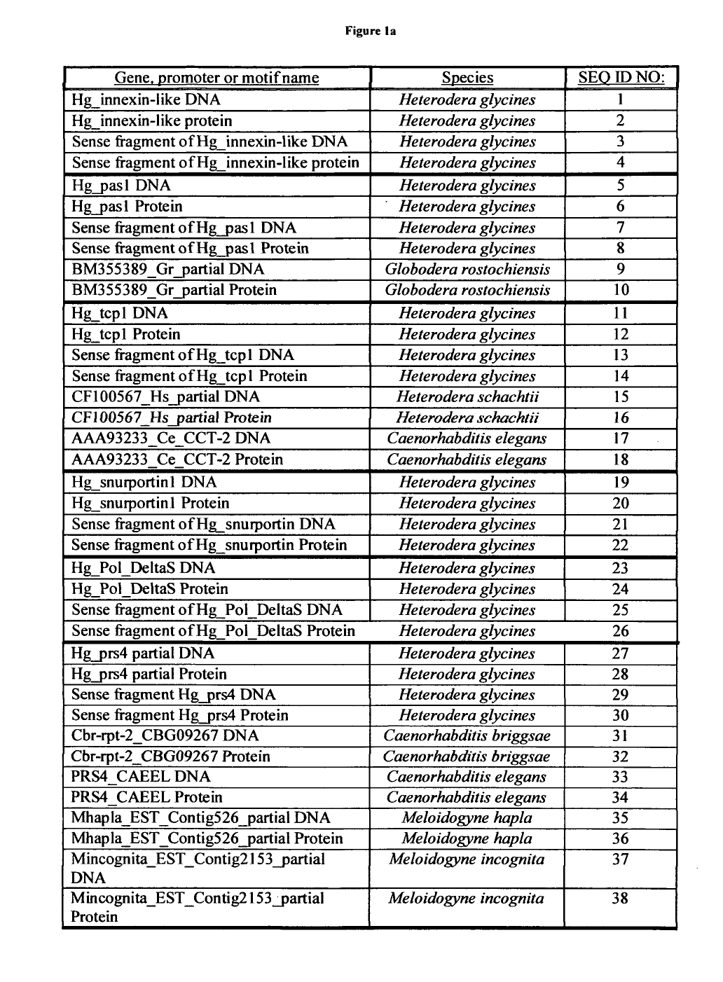

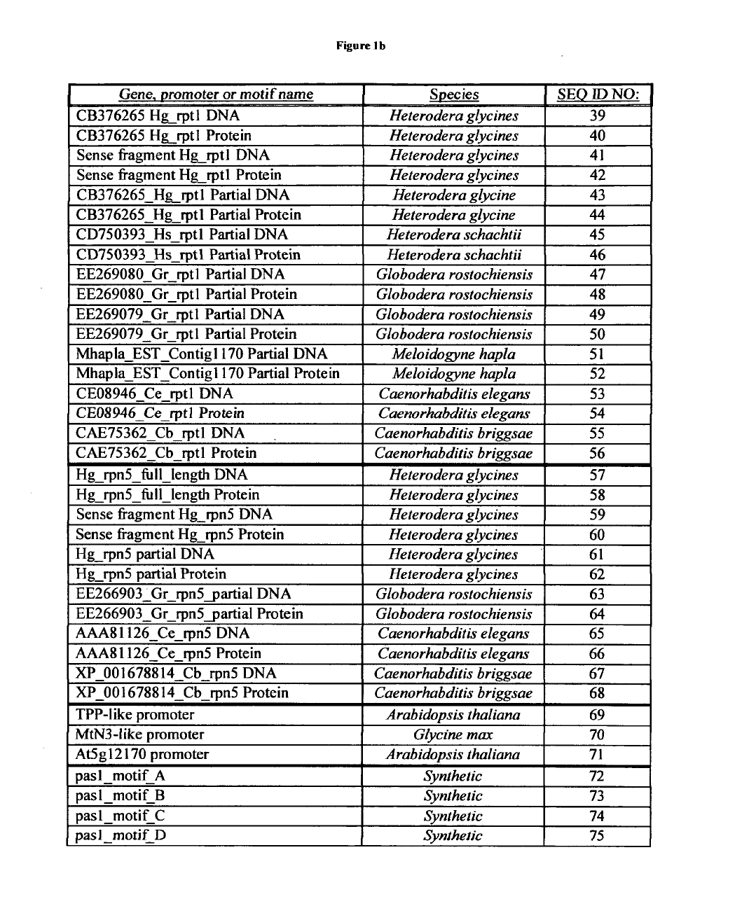

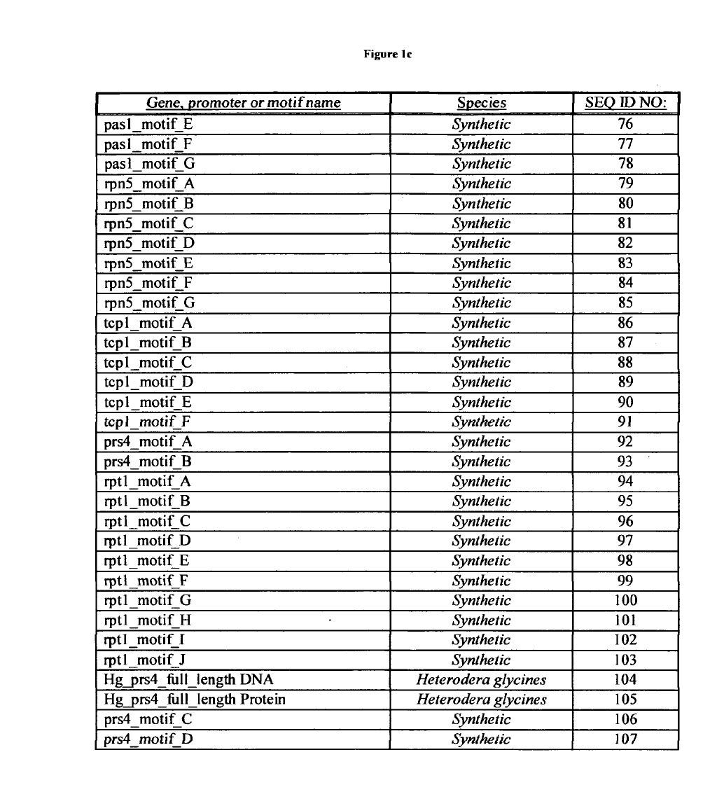

### DETAILED DESCRIPTION OF THE INVENTION

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized It is to be understood that the terminology used herein is for the purpose of describing specific embodiments only and is not intended to be limiting.

Throughout this application, various publications are referenced. The disclosures of all of these publications and those references cited within those publications in their entireties are hereby incorporated by reference into this application in order to more fully describe the state of the art to which this invention pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

As used herein, “RNAi” or “RNA interference” refers to the process of sequence-specific post-transcriptional gene silencing in nematodes, mediated by double-stranded RNA (dsRNA). As used herein, “dsRNA” refers to RNA that is partially or completely double stranded. Double stranded RNA is also referred to as short interfering RNA (siRNA), short interfering nucleic acid (siNA), micro-RNA (miRNA), and the like. In the RNAi process, dsRNA comprising a first strand that is substantially identical to a portion of a target gene and a second strand that is complementary to the first strand is introduced into a nematode, preferably by soaking and more preferably by feeding. After introduction into the nematode, the target gene-specific dsRNA is processed into relatively small fragments (siRNAs) and can subsequently become distributed from intestine to other parts of the nematode, leading to a loss-of-function mutation having a phenotype that, over the period of a generation, may come to closely resemble the phenotype arising from a complete or partial deletion of the target gene. Alternatively, the target gene-specific dsRNA is processed into relatively small fragments by a plant cell containing the RNAi processing machinery; and when the plant-processed small dsRNA is ingested by a parasitic nematode, the loss-of-function phenotype is obtained.

As used herein, taking into consideration the substitution of uracil for thymine when comparing RNA and DNA sequences, the term “substantially identical” as applied to dsRNA means that the nucleotide sequence of one strand of the dsRNA is at least about 80%-90% identical to 19 or more contiguous nucleotides of the target gene, more preferably, at least about 90-95% identical to 19 or more contiguous nucleotides of the target gene, and most preferably at least about 95%, 96%, 97%, 98% or 99% identical or absolutely identical to 19 or more contiguous nucleotides of the target gene. The term “19 or more contiguous nucleotides of the target gene” corresponds to the double-stranded portion of the dsRNA which is complementary to the target gene, being at least about 19, 20, 21, 22, 23, 24, 25, 50, 100, 200, 300, 400, 500, 1000, 1500, consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least at 80% of their nucleotides. Preferably, the two nucleic acid sequences are complementary over at least at 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the terms “contacting” and “administering” are used interchangeably, and refer to a process by which dsRNA of the present invention is delivered to a cell of a parasitic nematode, in order to inhibit expression of an essential target gene in the nematode. The dsRNA may be administered in a number of ways, including, but not limited to, direct introduction into a cell (i.e., intracellularly); or extracellular introduction into a cavity, interstitial space, or into the circulation of the nematode, oral introduction, the dsRNA may be introduced by bathing the nematode in a solution containing dsRNA, or the dsRNA may be present in food source. Methods for oral introduction include direct mixing of dsRNA with food of the nematode, as well as engineered approaches in which a species that is used as food is engineered to express a dsRNA, then fed to the organism to be affected. For example, the dsRNA may be sprayed onto a plant, or the dsRNA may be applied to soil in the vicinity of roots, taken up by the plant and/or the parasitic nematode, or a plant may be genetically engineered to express the dsRNA in an amount sufficient to kill or adversely affect some or all of the parasitic nematode to which the plant is exposed.

As used herein, the term “control,” when used in the context of an infection, refers to the reduction or prevention of an infection. Reducing or preventing an infection by a nematode will cause a plant to have increased resistance to the nematode; however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a dsRNA directed to the target gene. The plant's resistance to infection by the nematode may be due to the death, sterility, arrest in development, or impaired mobility of the nematode upon exposure to the dsRNA specific to an essential gene. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a parasitic nematode. As used herein, “inhibiting expression” refers to the absence or observable decrease in the level of protein and/or mRNA product from a target gene. Inhibition of target gene expression may be lethal to the parasitic nematode, or such inhibition may delay or prevent entry into a particular developmental step (e.g., metamorphosis), if plant disease is associated with a particular stage of the parasitic nematode's life cycle. The consequences of inhibition can be confirmed by examination of the outward properties of the nematode (as presented below in the examples).

In accordance with the invention, a parasitic nematode is contacted with a dsRNA, which specifically inhibits expression of a target gene that is essential for survival, metamorphosis, or reproduction of the nematode. Preferably, the parasitic nematode comes into contact with the dsRNA after entering a plant that expresses the dsRNA. In one embodiment, the dsRNA is encoded by a vector that has been transformed into an ancestor of the infected plant. Preferably, the nucleic acid sequence expressing said dsRNA is under the transcriptional control of a root specific promoter, a parasitic nematode induced feeding cell-specific promoter or a constitutive promoter.

In one embodiment, the parasitic nematode target gene is an innexin-like gene. Innexins comprise a large family of genes that are believed to encode invertebrate gap junction channel-forming proteins. These channel forming proteins allow for transport of ions and other small molecules between adjacent cells. In C. elegans, RNAi targeting innexins results in embryonic and larval lethality in C. elegans. Preferably, the target gene is a homolog of the C. elegans innexin gene family and is derived from a plant parasitic nematode. In this embodiment of the present invention, the parasitic nematode innexin-like target gene comprises a sequence selected from the group consisting of: (a) the sequences set forth in SEQ ID NO:1 or 3 and (b) a polynucleotide having at least 80% sequence identity to SEQ ID NO:1 or 3. As shown in Example 1, the full length H. glycines innexin-like gene was isolated and is represented in SEQ ID NO: 1.

In another embodiment, the parasitic nematode target gene is a gene encoding a polymerase delta small subunit (pol delta S). Polymerase delta is involved in DNA replication, repair, and recombination. The small subunit is non-catalytic. The small subunit is required for functional interaction of the catalytic subunit with proliferating cell nuclear antigen and processive DNA synthesis. In C. elegans, RNAi targeting polymerase delta small subunit (F12F6.7) results in embryonic lethality. Preferably, the target gene is a homolog of the C. elegans polymerase delta small subunit gene and is derived from a plant parasitic nematode. In this embodiment of the present invention, the parasitic nematode polymerase delta small subunit target gene comprises a sequence selected from the group consisting of: (a) the sequences set forth in SEQ ID NO:23 or 25 and (b) a polynucleotide having at least 80% sequence identity to SEQ ID NO:23 or 25. As shown in Example 1, the full length H. glycines polymerase delta small subunit gene was isolated and is represented in SEQ ID NO: 23.

In another embodiment, the parasitic nematode target gene is a homolog of the C. elegans tcp-1 gene T21B10.7 (Genbank accession AAA93233) which encodes a putative alpha subunit of the eukaryotic cytosolic (‘T complex’) chaperonin. This T-complex protein is required for normal pronuclear-centrosome rotation, positioning of the mitotic spindle, meiosis, and distal tip cell migration; it is also required for fertility and viability in the C. elegans. Preferably the target gene is a homolog of the C. elegans tcp-1 gene and is derived from a plant parasitic nematode. In this embodiment of the present invention, the parasitic nematode tcp-1 target gene comprises a sequence selected from the group consisting of: (a) the sequences set forth in SEQ ID NO:11 or 13 and (b) a polynucleotide having at least 80% sequence identity to SEQ ID NO:11 or 13. As shown in Example 1, the full length H. glycines tcp-1 like gene was isolated and is represented in SEQ ID NO: 11.

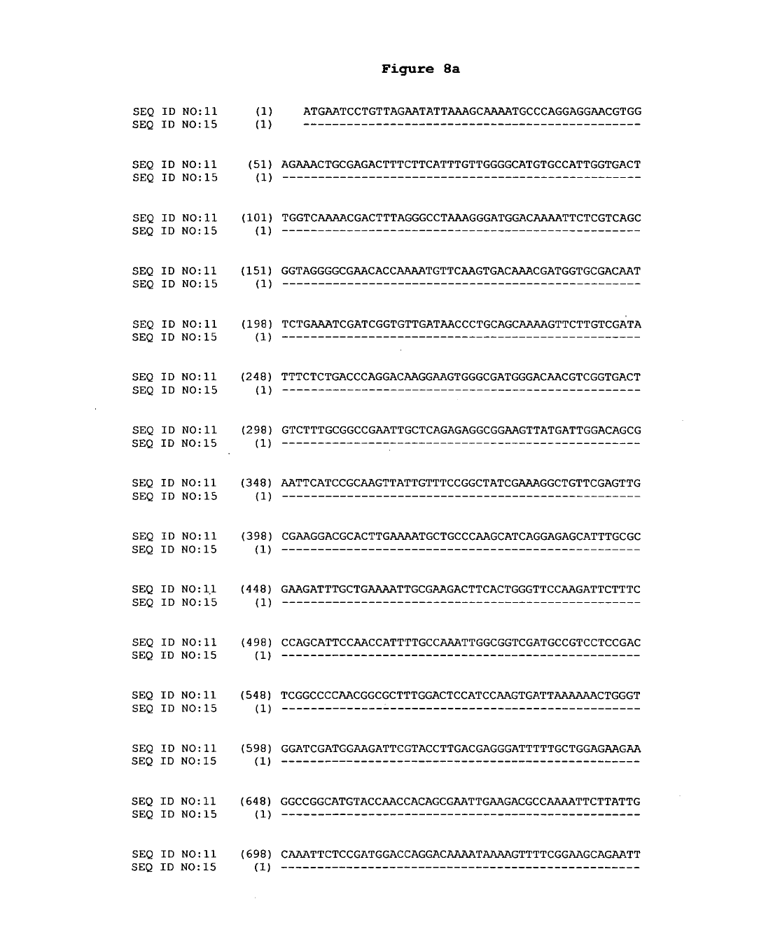

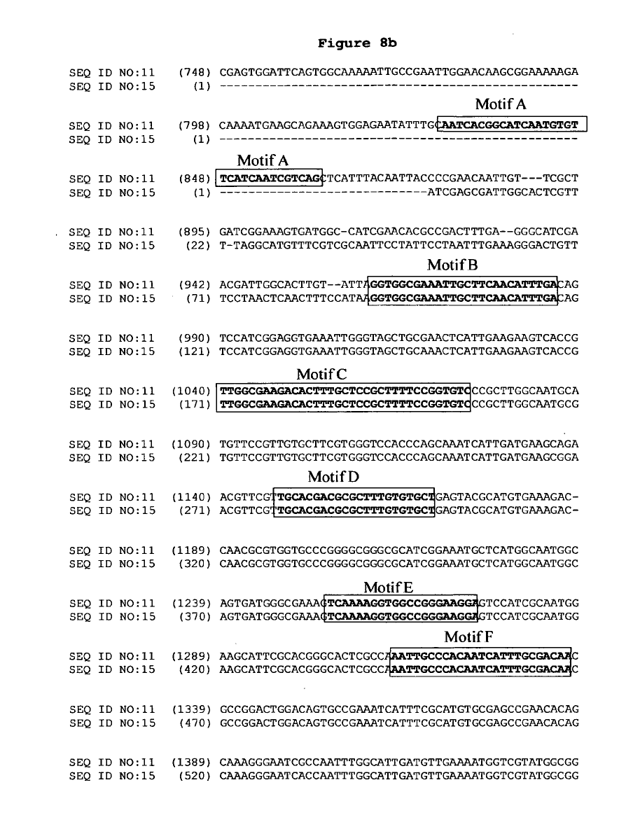

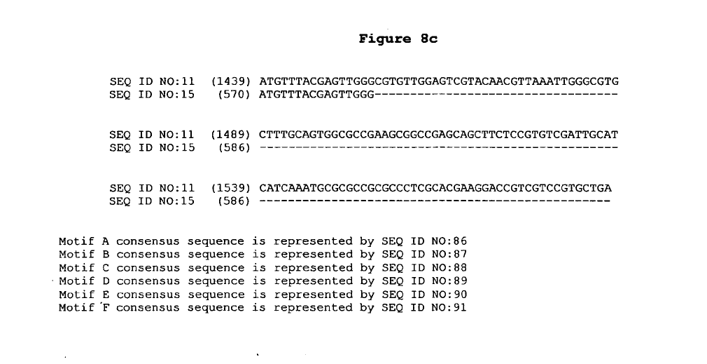

In another embodiment, the parasitic nematode target gene is a homolog of the C. elegans tcp-1 gene T21B10.7 (Genbank accession AAA93233) or a sequence fragment motif derived using the DNA sequence corresponding to amino acid sequence homologous to the C. elegans tcp-1 gene. As disclosed in Example 1, the full length transcript of the H. glycines tcp-1 like gene was isolated and is represented in SEQ ID NO:11. The sequence described by SEQ ID NO:11 contains an open reading frame with the amino acid sequence disclosed as SEQ ID NO:12. As disclosed in Example 4, the amino acid sequence described by SEQ ID NO:12 was used to identify homologous gene amino acid sequences. The corresponding homologous DNA sequence is described by SEQ ID NO:15. The DNA sequence alignment of the identified homolog described by SEQ ID NO:15 to SEQ ID NO:11 is shown in FIG. 8a-c. Regions of high sequence homology over 21 nucleotides or more are marked as Motif A through Motif F in FIG. 8a-c. The motif sequences corresponding to Motif A through Motif F are described by SEQ ID NOs 86-91. In this embodiment of the present invention, the homologous sequence or sequence fragment motif of the parasitic nematode tcp-1 target gene comprises a sequence selected from the group consisting of: (a) the sequence set forth in SEQ ID NO:15, (b) a polynucleotide having at least 80% sequence identity to SEQ ID NO:15, and (c) the sequences set forth in SEQ ID NO:86, 87, 88, 89, 90, or 91.

In another embodiment, the parasitic nematode target gene is a homolog of the C. elegans pas-1 gene which encodes a proteasome alpha subunit. Proteasome alpha subunits are part of the 26S proteasome's 20S protease core particle. They act as a gate through which tagged proteins enter the proteasome for degradation. Preferably, the target gene is a homolog of the C. elegans pas-1 gene and is derived from a plant parasitic nematode. In this embodiment of the present invention, the parasitic nematodepas-1 target gene comprises a sequence selected from the group consisting of: (a) the sequences set forth in SEQ ID NO:5 or 7 and (b) a polynucleotide having at least 80% sequence identity to SEQ ID NO:5 or 7. As shown in Example 1, the full length H. glycines pas-1 gene was isolated and is represented in SEQ ID NO: 5.

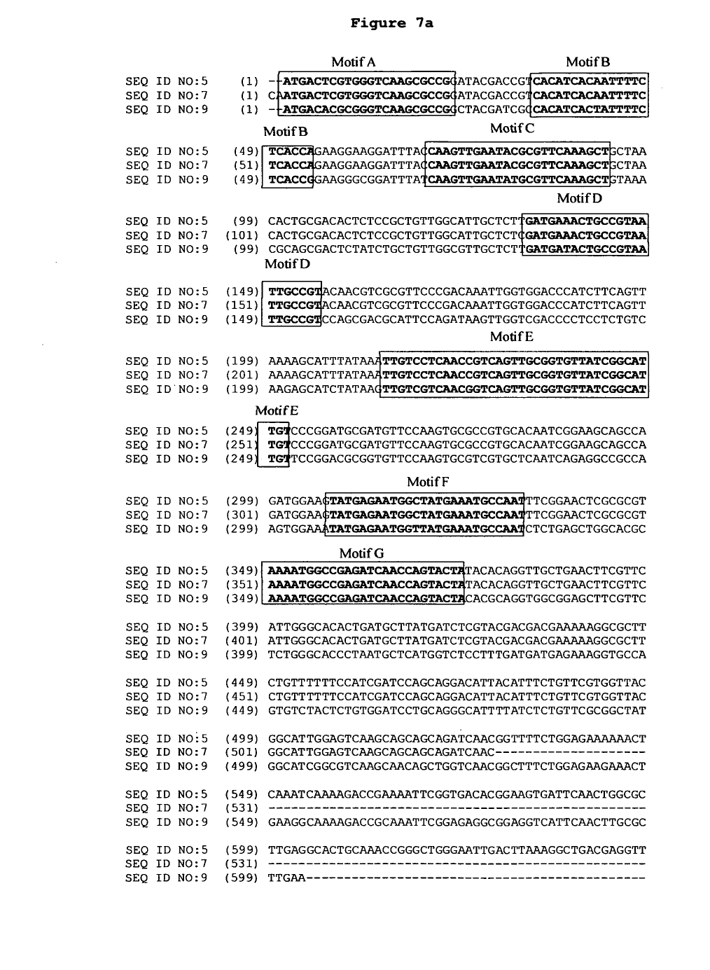

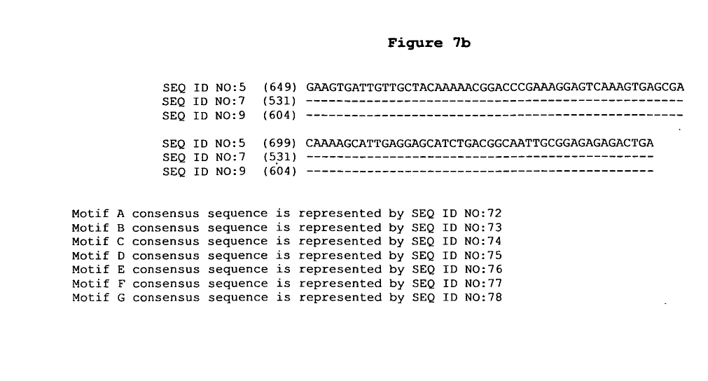

In another embodiment, the parasitic nematode target gene is a homolog of the C. elegans pas-1 gene or a sequence fragment motif derived using the DNA sequence corresponding to the amino acid sequence homologous to the C. elegans pas-1 gene. As disclosed in Example 1, the full length transcript of the H. glycines pas-1 like gene was isolated and is represented in SEQ ID NO:5. The sequence described by SEQ ID NO:5 contains an open reading frame with the amino acid sequence disclosed as SEQ ID NO:6. As disclosed in Example 4, the amino acid sequence described by SEQ ID NO:6 was used to identify homologous gene amino acid sequences. The corresponding homologous DNA sequence is described by SEQ ID NO:9. The DNA sequence alignment of the identified homolog described by SEQ ID NO:9 to SEQ ID NO:5 is shown in FIG. 7a-b. Regions of high sequence homology over 21 nucleotides or more are marked as Motif A through Motif G in FIG. 7a-b. The motif sequences corresponding to Motif A through Motif G are described by SEQ ID NOs 72-78. In this embodiment of the present invention, the homolous sequence or sequence fragment motif of the parasitic nematodepas-1 target gene comprises a sequence selected from the group consisting of: (a) the sequence set forth in SEQ ID NO:9, (b) a polynucleotide having at least 80% sequence identity to SEQ ID NO:9, and (c) the sequences set forth in SEQ ID NO:72, 73, 74, 75, 76, 77, or 78.

In another embodiment, the parasitic nematode target is a parasitic nematode snurportin-1 like gene. Snurportins are nuclear import receptors involved in importing m3G-capped U snRNPs (Small nuclear ribonucleoprotein), used for splicing, into the nucleus. In C. elegans, RNAi targeting snurportin-1 (F23F1.5) results in embryonic lethality. Preferably, the target gene is a homolog of the C. elegans snurportin-1 gene F23G1.5 (Genbank accession AAB70323) and is derived from a plant parasitic nematode. In this embodiment of the present invention, the parasitic nematode snurportin-1 target gene comprises a sequence selected from the group consisting of: (a) the sequences set forth in SEQ ID NO:19 or 21 and (b) a polynucleotide having at least 80% sequence identity to SEQ ID NO:19 or 21. As shown in Example 1, the full length H. glycines snurportin-1 gene was isolated and is represented in SEQ ID NO: 19.

In another embodiment, the parasitic nematode target gene is a homolog of the C. elegans rpt-1 gene which encodes a predicted ATPase subunit of the 19S regulatory complex of the proteasome that affects fertility and embryonic viability. Preferably, the target gene is a homolog of the C. elegans rpt-1 gene C52E4.4 (EMBL accession CAB01414) and is derived from a plant parasitic nematode. In this embodiment of the present invention, the parasitic nematode rpt-1 target gene comprises a sequence selected from the group consisting of: (a) the sequences set forth in SEQ ID NO:39, 41 or 43 and (b) a polynucleotide having at least 80% sequence identity to SEQ ID NO: 39, 41 or 43. As shown in Example 1, the full length H. glycines rpt-1 like gene was isolated and is represented in SEQ ID NO: 39.

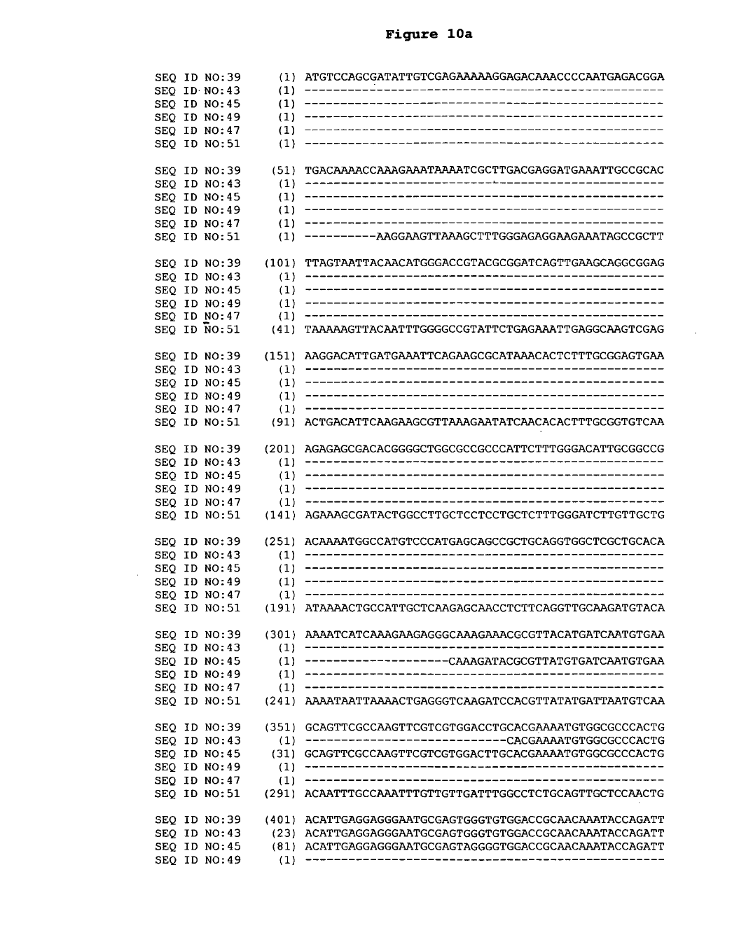

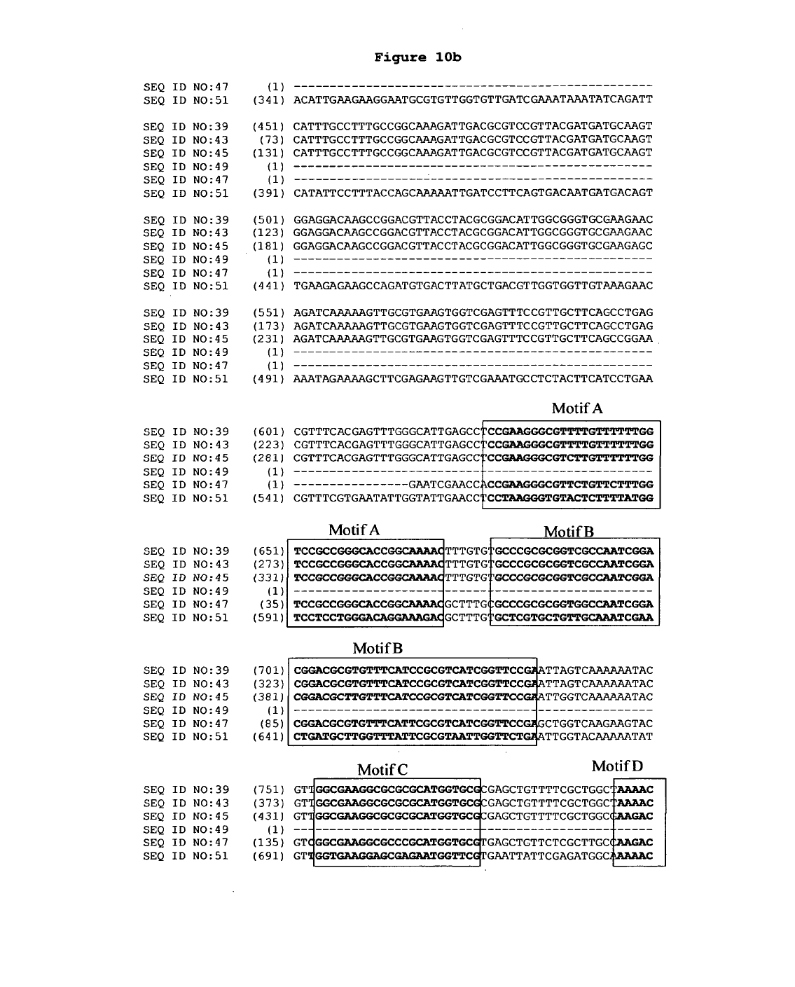

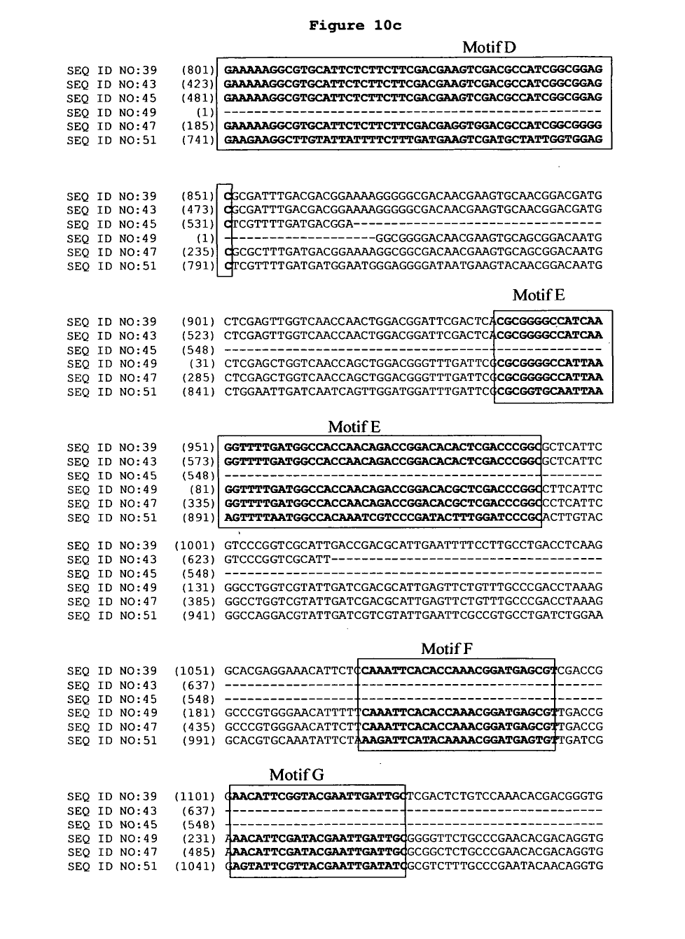

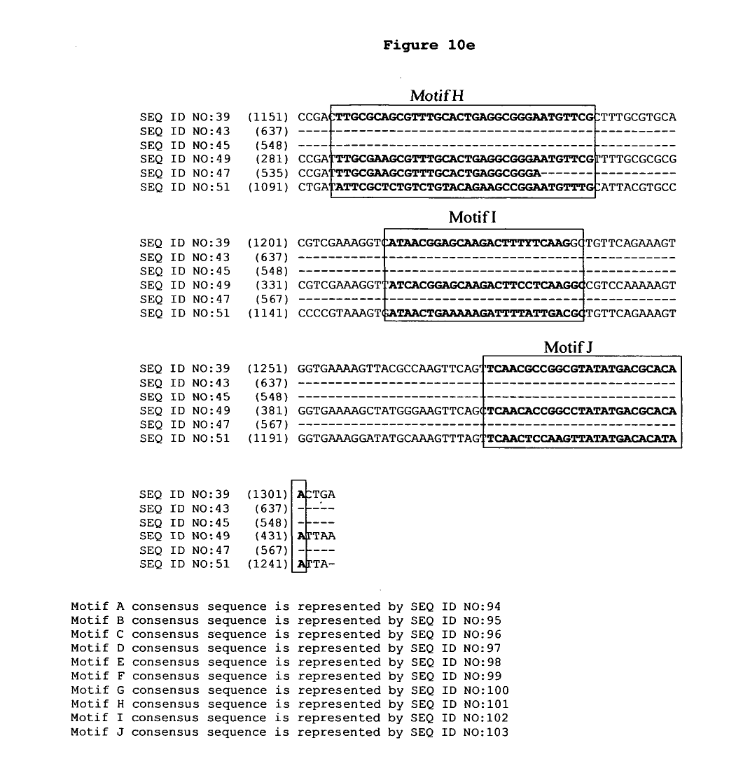

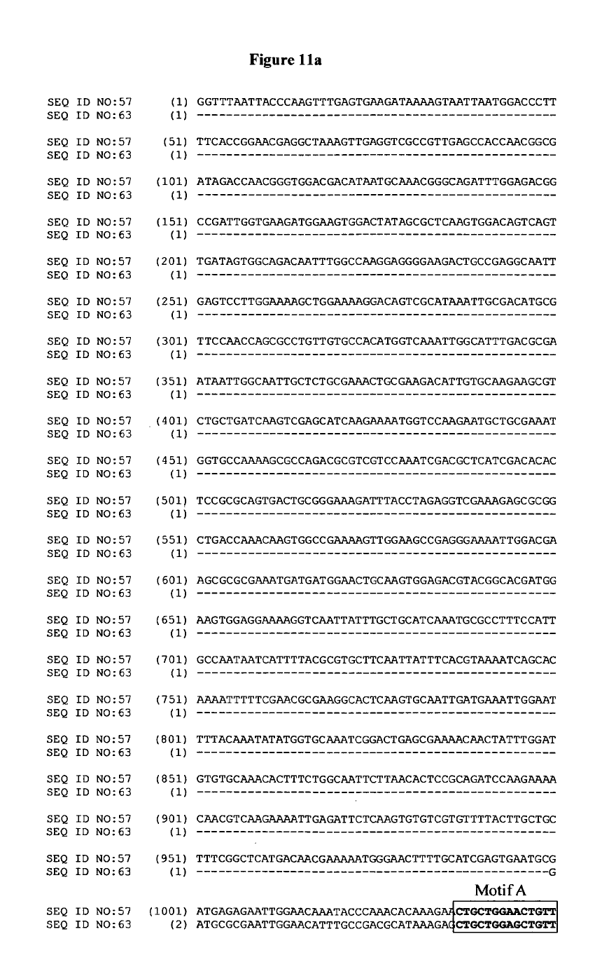

In another embodiment, the parasitic nematode target gene is a homolog of the C. elegans rpt-1 gene C52E4.4 (EMBL accession CAB01414) or a sequence fragment motif derived using the DNA sequence corresponding to the amino acid sequence homologous to the C. elegans rpt-1 gene. As disclosed in Example 1, the full length transcript of the H. glycines rpt-1 like gene was isolated and is represented in SEQ ID NO:39. The sequence described by SEQ ID NO:39 contains an open reading frame with the amino acid sequence disclosed as SEQ ID NO:40. As disclosed in Example 4, the amino acid sequence described by SEQ ID NO:40 was used to identify homologous gene amino acid sequences. The corresponding homologous DNA sequences are described by SEQ ID NO:45, 47, 49, 51, 53, and 55. The DNA sequence alignment of the identified plant parasitic nematode homologs described by SEQ ID NO:45, 47, 49, and 51 to SEQ ID NO:39 is shown in FIG. 10a-e. Regions of high sequence homology over 21 nucleotides or more are marked as Motif A through Motif J in FIG. 10a-e. The motif sequences corresponding to Motif A through Motif J are described by SEQ ID NOs 94-103. In this embodiment of the present invention, the homolous sequences or sequence fragment motifs of the parasitic nematode rpt-1 target gene comprises a sequence selected from the group consisting of: (a) the sequence set forth in SEQ ID NO:45, 47, 49, or 51, (b) a polynucleotide having at least 80% sequence identity to SEQ ID NO:45, 47, 49, or 51, and (c) the sequences set forth in SEQ ID NO:94, 95, 96, 97, 98, 99, 100, 101, 102, or 103.

In another embodiment, target is a gene encoding a parasitic nematode 26S proteasome regulatory subunit 4 (prs-4). The subunit 4 protein is part of the 19S regulatory complex of the 26S proteasome and contains an ATPase domain. Disruption of this gene in parasitic nematodes with RNAi would lead to potential defects in the proteasome and death. Preferably, the target gene is a homolog of the C. elegans 26S proteasome regulatory subunit 4 gene gene F29G9.5, Swiss-Prot entry 016368 and is derived from a plant parasitic nematode. In this embodiment of the present invention, the parasitic nematode 26S proteasome regulatory subunit 4 target gene comprises a sequence selected from the group consisting of: (a) the sequence set forth in SEQ ID NO:27, 29 or 104, (b) a polynucleotide having at least 80% sequence identity to SEQ ID NO:27, 29 or 104 and (c) a polynucleotide from a parasitic nematode that hybridizes under stringent conditions to the sequence set forth in SEQ ID NO:27, 29 or 104. As shown in Example 1, a full length H. glycines 26S proteasome regulatory subunit 4 gene sequence was isolated and is represented in SEQ ID NO: 104.

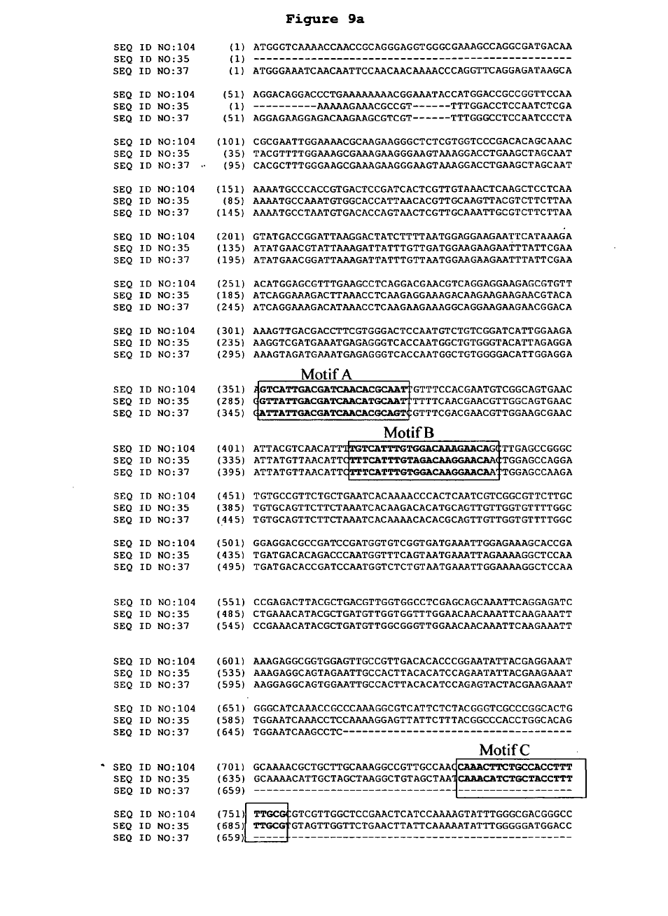

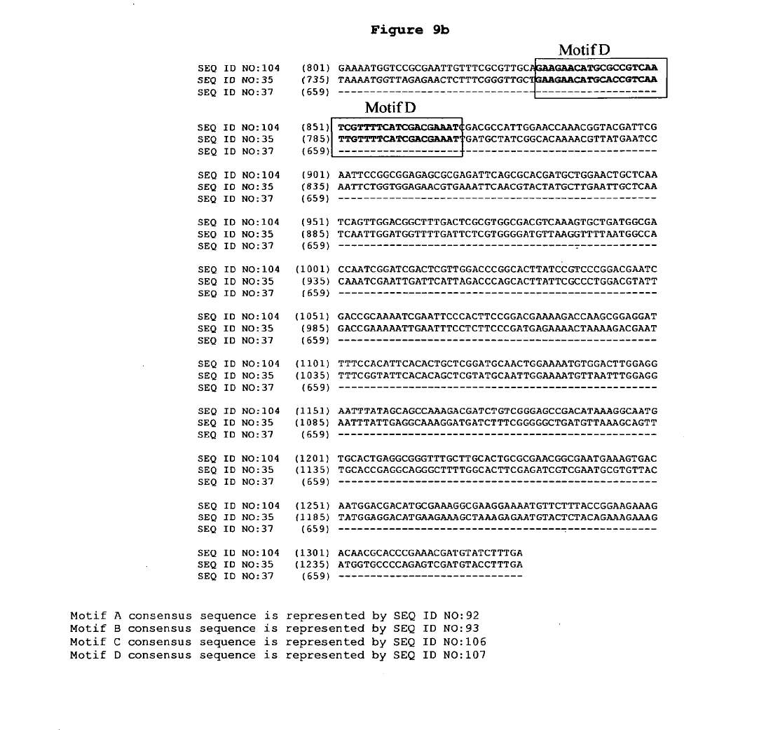

In another embodiment, the parasitic nematode target gene is a parasitic nematode 26S proteasome regulatory subunit 4 (prs-4) or a sequence fragment motif derived using the DNA sequence corresponding to the amino acid sequence homologous to the parasitic nematode 26S proteasome regulatory subunit 4 (prs-4) sequence. As disclosed in Example 1, a full length H. glycines 26S proteasome regulatory subunit 4 gene sequence was isolated and is represented in SEQ ID NO:104. The sequence described by SEQ ID NO:104 contains an open reading frame with the amino acid sequence disclosed as SEQ ID NO:105. As disclosed in Example 4, the amino acid sequence described by SEQ ID NO:105 was used to identify homologous gene amino acid sequences. The corresponding homologous DNA sequences are described by SEQ ID NO:31, 33, 35, and 37. The DNA sequence alignment of the identified homologs described by SEQ ID NO:35 and SEQ ID NO:37 to SEQ ID NO:104 is shown in FIG. 9a-b. Regions of high sequence homology over 21 nucleotides or more are marked as Motif A through Motif D in FIG. 9a-b. The motif sequences corresponding to Motif A through Motif D are described by SEQ ID NO:92, 93, 106, and 107. In this embodiment of the present invention, the homologous sequence or sequence fragment motif of the parasitic nematode prs-4 target gene comprises a sequence selected from the group consisting of: (a) the sequence set forth in SEQ ID NO:35 or 37, (b) a polynucleotide having at least 80% sequence identity to SEQ ID NO: 35 or 37, and (c) the sequences set forth in SEQ ID NO:92, 93, 106, or 107.

In another embodiment, the parasitic nematode target gene is a homolog of the C. elegans rpn-5 gene which encodes a proteasome regulatory particle. The protein is part of the 26S proteasome regulatory complex and contains a non-ATPase domain. RNAi studies in C. elegans feeding assays have shown embryonic lethal phenotypes. Preferably, the target gene is a homolog of the C. elegans rpn-5 gene F1OG7.8 (Genbank accession AAA81126) and is derived from a plant parasitic nematode. In this embodiment of the present invention, the parasitic nematode rpn-5 gene comprises a sequence selected from the group consisting of: (a) the sequences set forth in SEQ ID NO:57, 59 or 61, (b) a polynucleotide having at least 80% sequence identity to SEQ ID NO: 57, 59 or 61 and (c) a polynucleotide from a parasitic nematode that hybridizes under stringent conditions to the sequence set forth in SEQ ID NO: 57, 59 or 61. As shown in Example 1, the full length H. glycines rpn-5 gene was isolated and is represented in SEQ ID NO: 57.

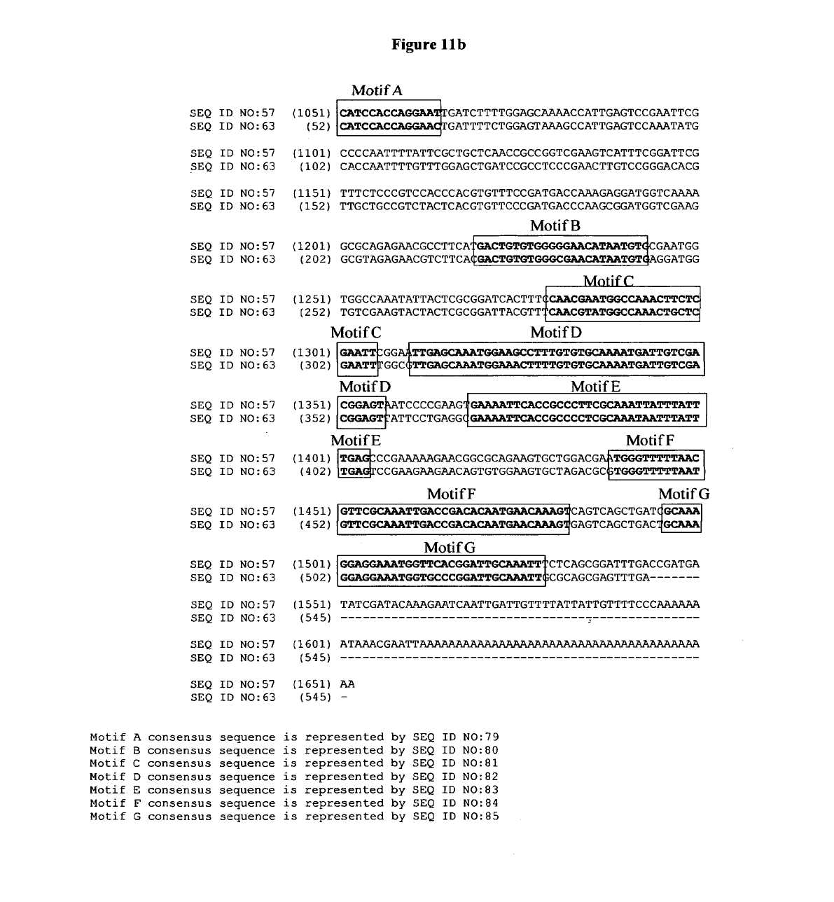

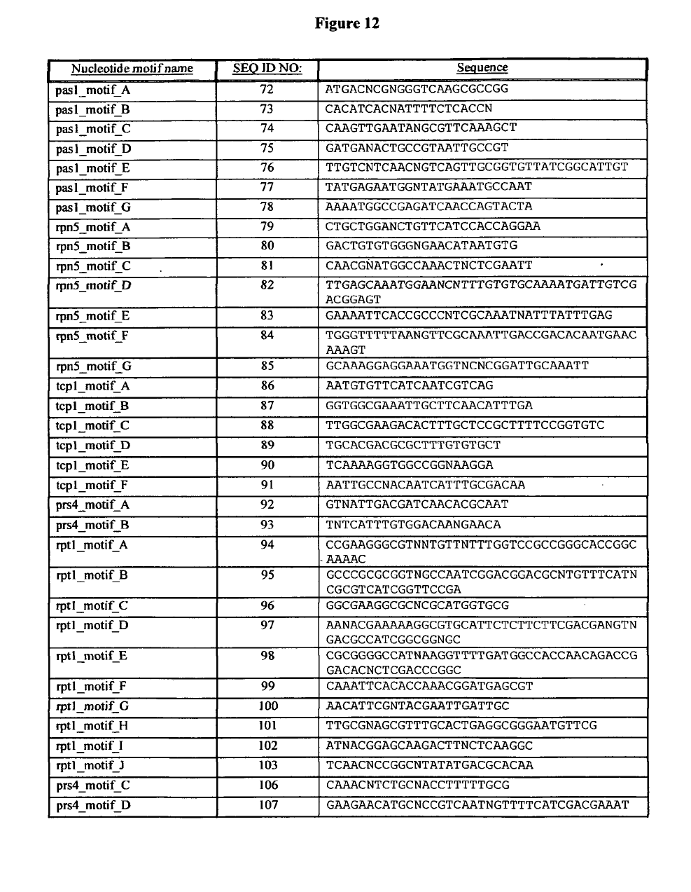

In another embodiment, the parasitic nematode target gene is a parasitic nematode rpn-5 gene or a sequence fragment motif derived using the DNA sequence corresponding to the amino acid sequence homologous to the parasitic nematode rpn-5 gene. As disclosed in Example 1, a full length H. glycines rpn-5 gene was isolated and is represented in SEQ ID NO:57. The sequence described by SEQ ID NO:57 contains an open reading frame with the amino acid sequence disclosed as SEQ ID NO:58. As disclosed in Example 4, the amino acid sequence described by SEQ ID NO:58 was used to identify homologous gene amino acid sequences. The corresponding homologous DNA sequence is described by SEQ ID NO:63. The DNA sequence alignment of the identified homolog described by SEQ ID NO:63 to SEQ ID NO:57 is shown in FIG. 11a-b. Regions of high sequence homology over 21 nucleotides or more are marked as Motif A through Motif G in FIG. 11a-b. The motif sequences corresponding to Motif A through Motif G are described by SEQ ID NOs 79-85. In this embodiment of the present invention, the homologous sequence or sequence fragment motif of the parasitic nematode rpn-5 target gene comprises a sequence selected from the group consisting of: (a) the sequence set forth in SEQ ID NO:63, (b) a polynucleotide having at least 80% sequence identity to SEQ ID NO:63, and (c) the sequences set forth in SEQ ID NO:79, 80, 81, 82, 83, 84, or 85.

Complete cDNAs corresponding to the parasitic nematode target genes of the invention may be isolated from parasitic nematodes other than H. glycines using the information provided herein and techniques known to those of skill in the art of biotechnology. For example, a nucleic acid molecule from a parasitic nematode that hybridizes under stringent conditions to a nucleotide sequence of SEQ ID NO:1, 3, 5, 7, 11, 13, 19, 21, 23, 25, 27, 104, 29, 39, 41, 43, 57, 59 or 61 can be isolated from parasitic nematode cDNA libraries. As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; well known in the art. Alternatively, mRNA can be isolated from parasitic nematode cells, and cDNA can be prepared using reverse transcriptase. Synthetic oligonucleotide primers for polymerase chain reaction amplification can be designed based upon the nucleotide sequence shown in SEQ ID NO:1, 3, 5, 7, 11, 13, 19, 21, 23, 25, 27, 104, 29, 39, 41, 43, 57, 59 or 61. Nucleic acid molecules corresponding to the parasitic nematode target genes of the invention can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

Accordingly, in one embodiment the dsRNA of the invention comprises a first strand that is substantially identical to a portion of the innexin-like target gene of a plant parasitic nematode genome and a second strand that is substantially complementary to the first strand. In preferred embodiments, the target gene is selected from the group consisting of: (a) a polynucleotide having the sequence set forth in SEQ ID NO:1 or 3; (b) a polynucleotide having at least 80% sequence identity to SEQ ID NO:1 or 3; and (c) a polynucleotide from a parasitic nematode that hybridizes under stringent conditions to a polynucleotide having the sequence set forth in SEQ ID NO:1 or 3.

In another embodiment, the dsRNA of the invention comprises a first strand that is substantially identical to a portion of the pas-1 target gene of a plant parasitic nematode genome and a second strand that is substantially complementary to the first strand. In preferred embodiments, the target gene is selected from the group consisting of: (a) a polynucleotide having the sequence set forth in SEQ ID NO:5, 7, 9, 72, 73, 74, 75, 76, 77, or 78; (b) a polynucleotide having at least 80% sequence identity to SEQ ID NO:5, 7, 9, 72, 73, 74, 75, 76, 77, or 78; and (c) a polynucleotide from a parasitic nematode that hybridizes under stringent conditions to a polynucleotide having the sequence set forth in SEQ ID NO:5, 7, 9, 72, 73, 74, 75, 76, 77, or 78.

In another embodiment, the dsRNA of the invention comprises a first strand that is substantially identical to a portion of the tcp-1 target gene of a plant parasitic nematode genome and a second strand that is substantially complementary to the first strand. In preferred embodiments, the target gene is selected from the group consisting of: (a) a polynucleotide having the sequence set forth in SEQ ID NO:11, 13, 15, 86, 87, 88, 89, 90, or 91; (b) a polynucleotide having at least 80% sequence identity to SEQ ID NO:11, 13, 15, 86, 87, 88, 89, 90, or 91; and (c) a polynucleotide from a parasitic nematode that hybridizes under stringent conditions to a polynucleotide having the sequence set forth in SEQ ID NO:11, 13, 15, 86, 87, 88, 89, 90, or 91.

In another embodiment, the dsRNA of the invention comprises a first strand that is substantially identical to a portion of the snurportin-1 like target gene of a plant parasitic nematode genome and a second strand that is substantially complementary to the first strand. In preferred embodiments, the target gene is selected from the group consisting of: (a) a polynucleotide having the sequence set forth in SEQ ID NO:19 or 21; (b) a polynucleotide having at least 80% sequence identity to SEQ ID NO: 19 or 21; and (c) a polynucleotide from a parasitic nematode that hybridizes under stringent conditions to a polynucleotide having the sequence set forth in SEQ ID NO: 19 or 21.

In another embodiment, the dsRNA of the invention comprises a first strand that is substantially identical to a portion of the polymerase delta small subunit target gene of a plant parasitic nematode genome and a second strand that is substantially complementary to the first strand. In preferred embodiments, the target gene is selected from the group consisting of: (a) a polynucleotide having the sequence set forth in SEQ ID NO:23 or 25; (b) a polynucleotide having at least 80% sequence identity to SEQ ID NO: 23 or 25; and (c) a polynucleotide from a parasitic nematode that hybridizes under stringent conditions to a polynucleotide having the sequence set forth in SEQ ID NO: 23 or 25.

In another embodiment, the dsRNA of the invention comprises a first strand that is substantially identical to a portion of the prs-4 target gene of a plant parasitic nematode genome and a second strand that is substantially complementary to the first strand. In preferred embodiments, the target gene is selected from the group consisting of: (a) a polynucleotide having the sequence set forth in SEQ ID NO:27, 104, 29, 92, 93, 106, or 107; (b) a polynucleotide having at least 80% sequence identity to SEQ ID NO: 27, 104, 29, 92, 93, 106, or 107; and (c) a polynucleotide from a parasitic nematode that hybridizes under stringent conditions to a polynucleotide having the sequence set forth in SEQ ID NO: 27, 104, 29, 92, 93, 106, or 107.

In another embodiment, the dsRNA of the invention comprises a first strand that is substantially identical to a portion of the rpt-1 target gene of a plant parasitic nematode genome and a second strand that is substantially complementary to the first strand. In preferred embodiments, the target gene is selected from the group consisting of: (a) a polynucleotide having the sequence set forth in SEQ ID NO:39, 41, 43, 94, 95, 96, 97, 98, 99, 100, 101, 102, or 103; (b) a polynucleotide having at least 80% sequence identity to SEQ ID NO:39, 41, 43, 94, 95, 96, 97, 98, 99, 100, 101, 102, or 103; and (c) a polynucleotide from a parasitic nematode that hybridizes under stringent conditions to a polynucleotide having the sequence set forth in SEQ ID NO: 39, 41, 43, 94, 95, 96, 97, 98, 99, 100, 101, 102, or 103.

In another embodiment, the dsRNA of the invention comprises a first strand that is substantially identical to a portion of the rpn-5 target gene of a plant parasitic nematode genome and a second strand that is substantially complementary to the first strand. In preferred embodiments, the target gene is selected from the group consisting of: (a) a polynucleotide having the sequence set forth in SEQ ID NO:57, 59, 61, 63, 79, 80, 81, 82, 83, 84, or 85; (b) a polynucleotide having at least 80% sequence identity to SEQ ID NO: 57, 59, 61, 63, 79, 80, 81, 82, 83, 84, or 85; and (c) a polynucleotide from a parasitic nematode that hybridizes under stringent conditions to a polynucleotide having the sequence set forth in SEQ ID NO: : 57, 59, 61, 63, 79, 80, 81, 82, 83, 84, or 85.

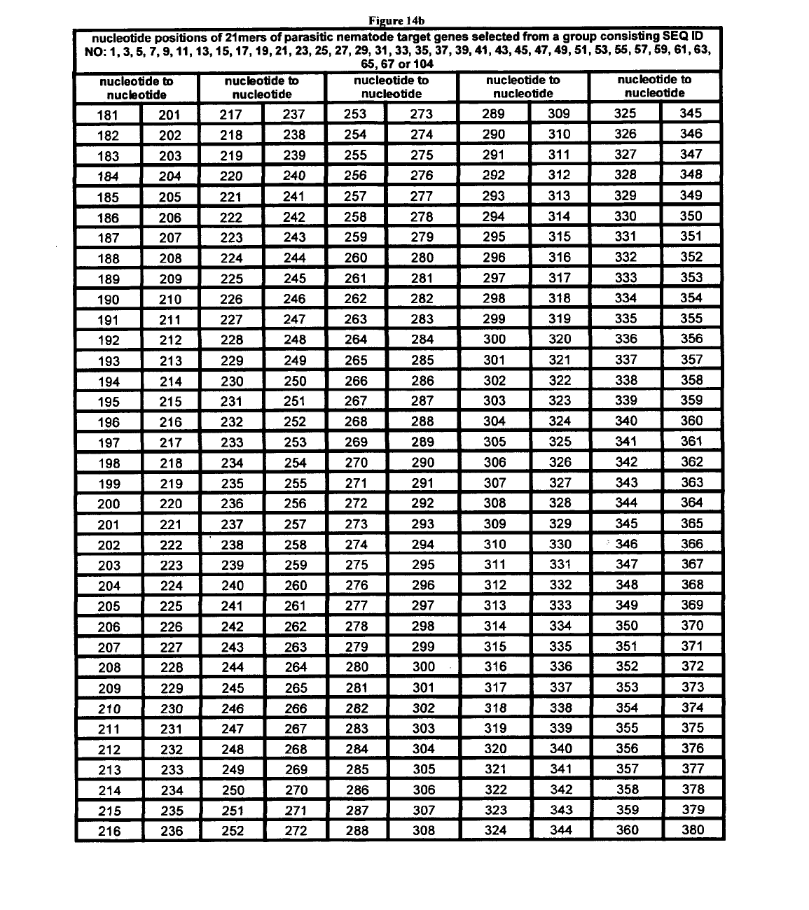

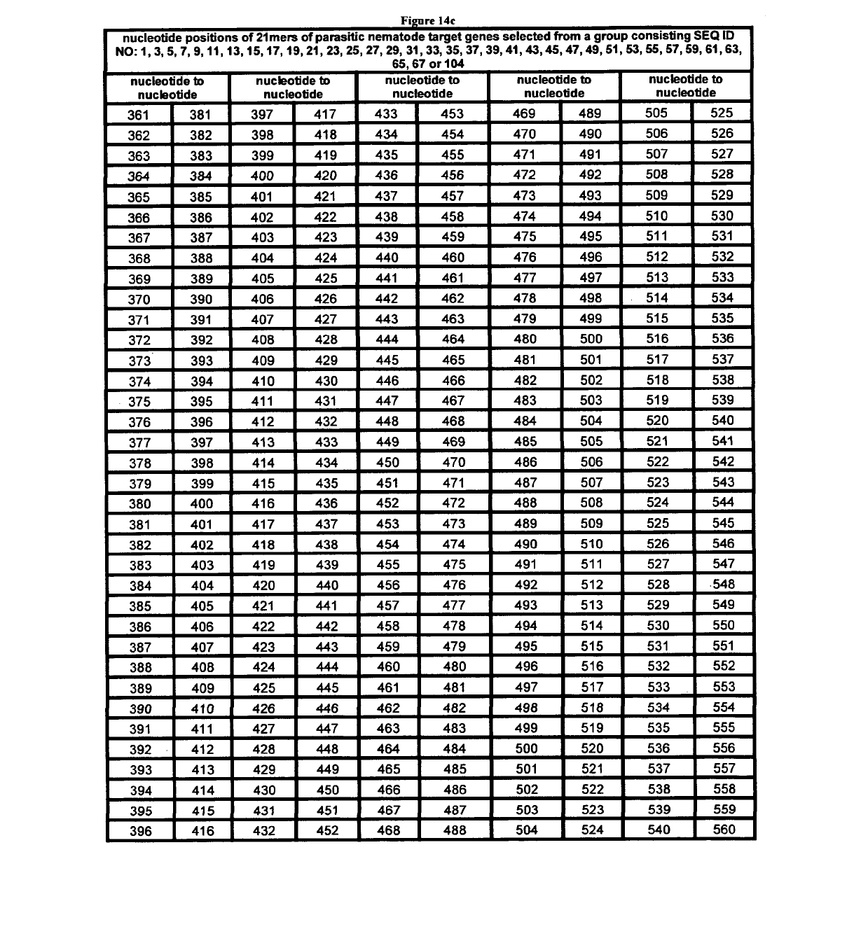

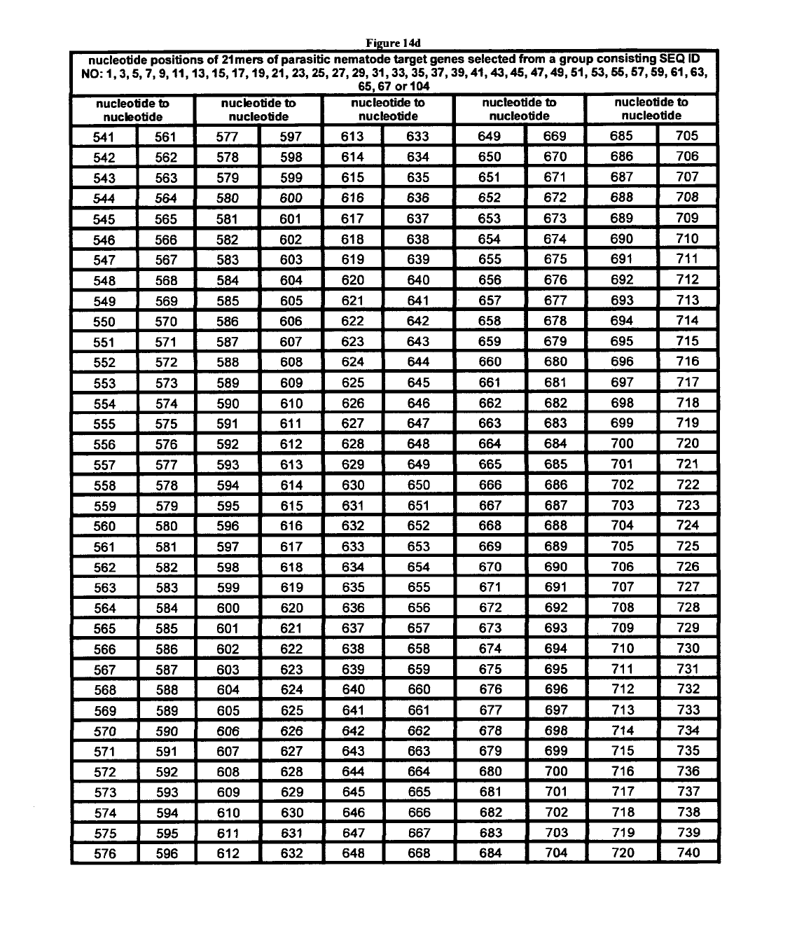

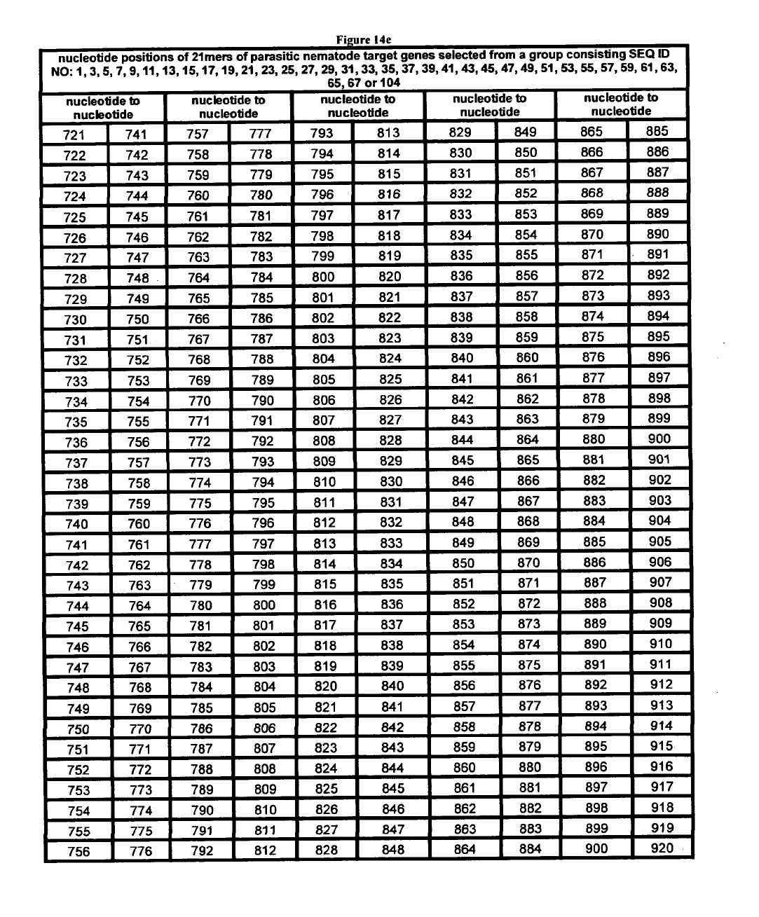

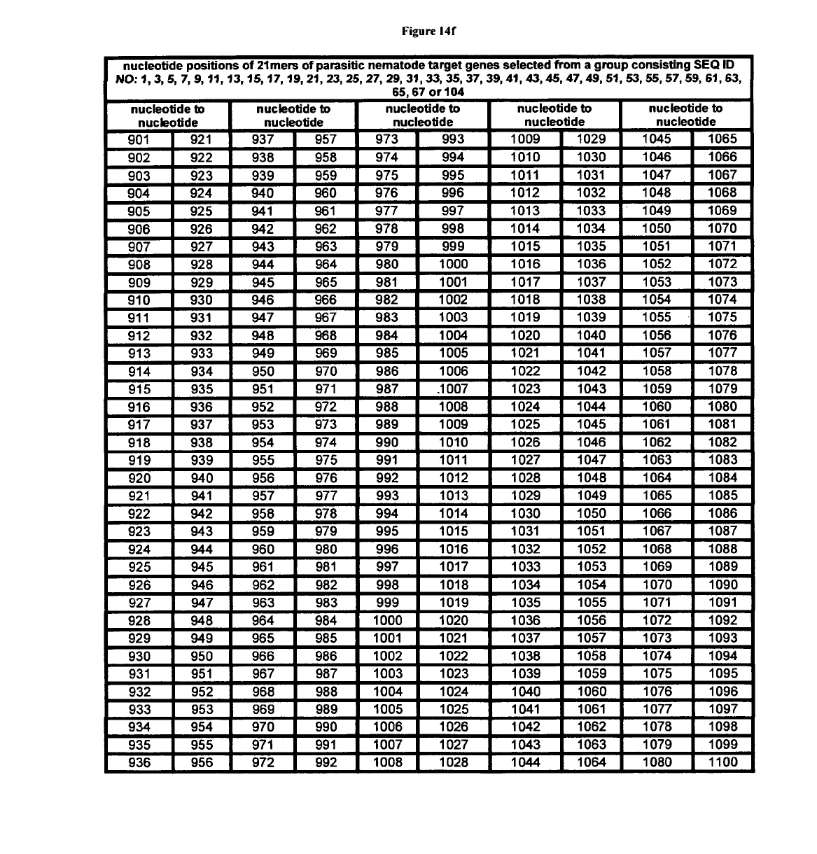

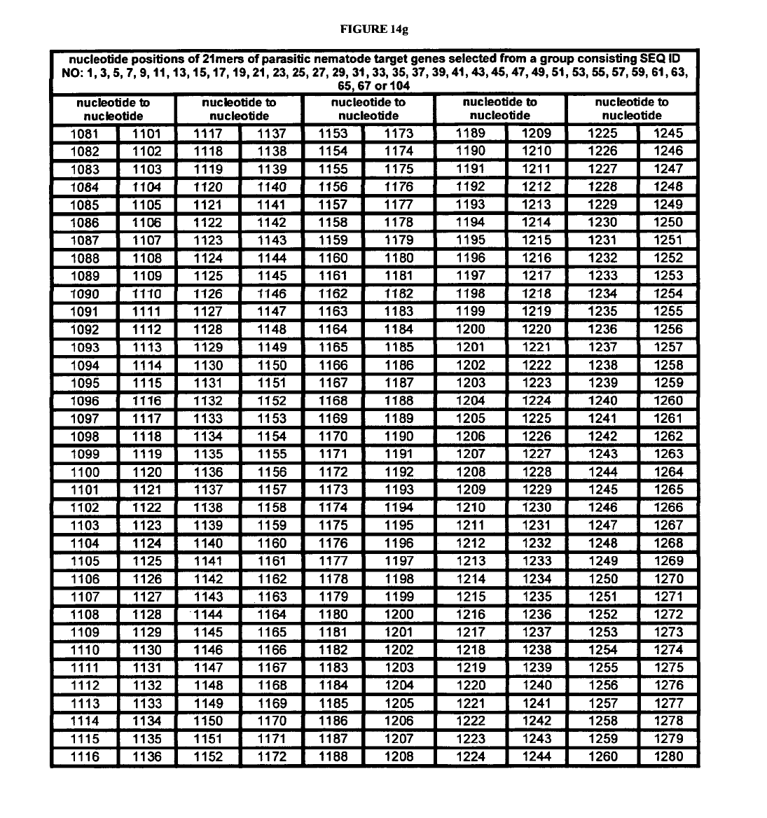

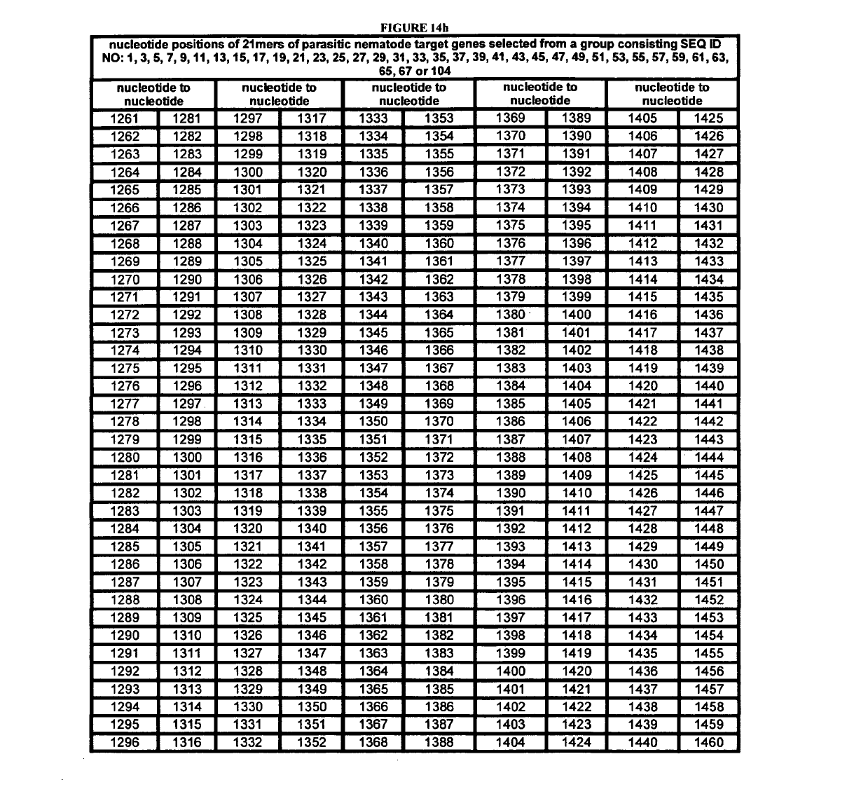

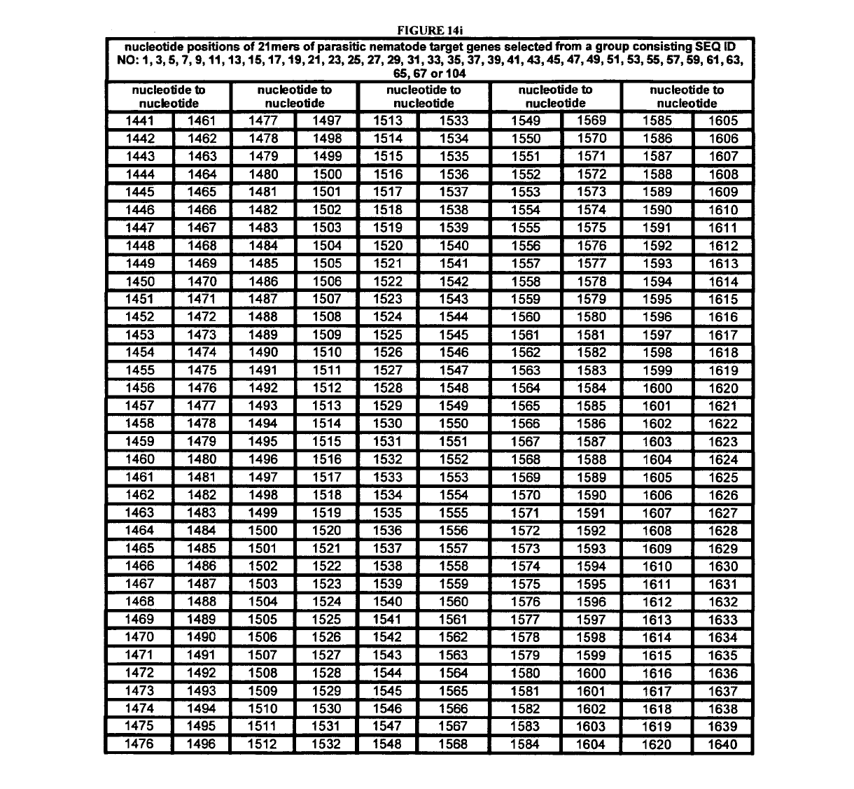

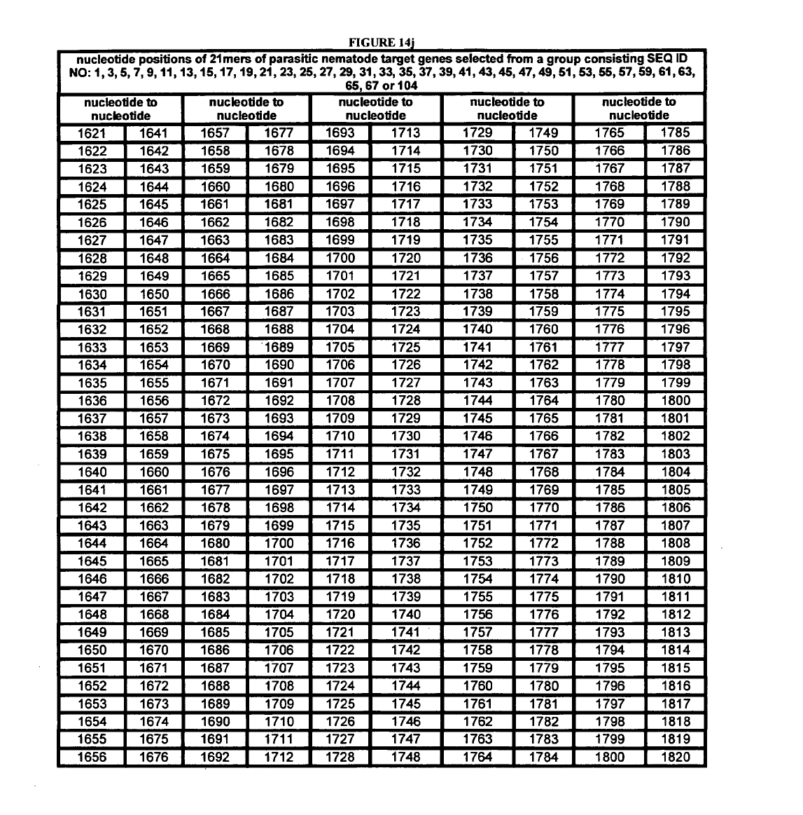

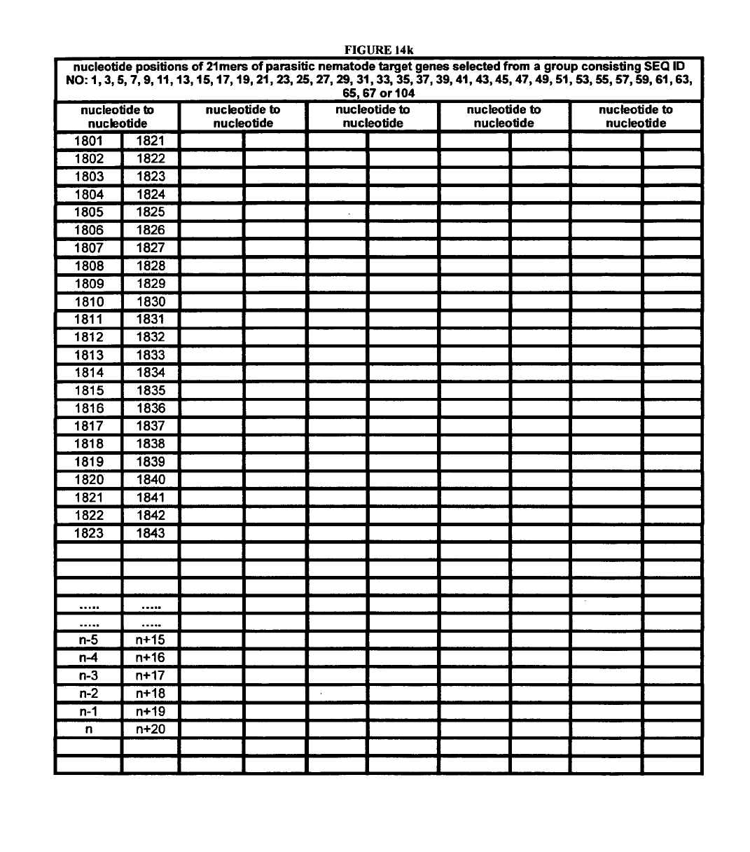

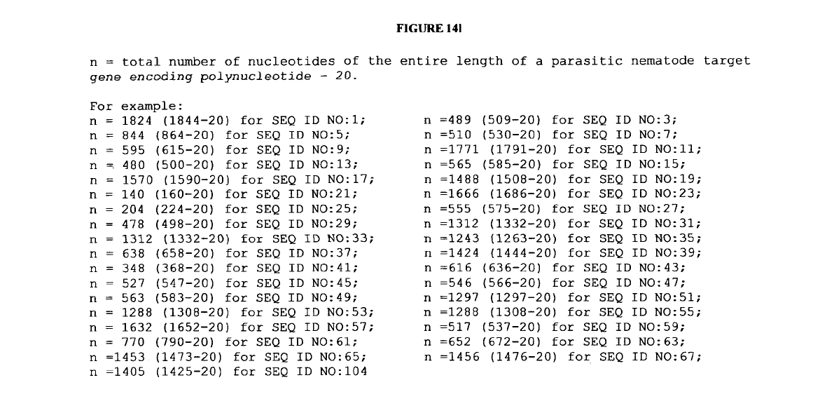

As discussed above, fragments of dsRNA larger than 19-24 nucleotides in length are cleaved intracellularly by nematodes and plants to siRNAs of 19-24 nucleotides in length, and these siRNAs are the actual mediators of the RNAi phenomenon. The table in FIGS. 14a-14l sets forth exemplary 21-mers of the SCN innexin-like gene, SEQ ID NO:1, pas-1 gene, SEQ ID NO:5, tcp-1 gene, SEQ ID NO:11, snurportin-1 like gene, SEQ ID NO:19,pol delta S gene, SEQ ID NO:23, prs-4 gene, SEQ ID NO:104, rpt-1 gene, SEQ ID NO:39, and rpn-5 gene, SEQ ID NO:57.and the respective fragments and homologs thereof, as indicated by SEQ ID NOs set forth in the table. This table can also be used to calculate the 19, 20, 22, 23, or 24-mers by adding or subtracting the appropriate number of nucleotides from each 21mer. Thus the dsRNA of the present invention may range in length from 19 nucleotides to about 500 consecutive nucleotides or up to the whole length of the target gene. The dsRNA of the invention may be embodied as a miRNA which targets a single site within a parasitic nematode target gene. Alternatively, the dsRNA of the invention has a length from about 19 nucleotides to about 600 consecutive nucleotides. In another embodiment, the dsRNA of the invention has a length from about 20 nucleotides to about 400 consecutive nucleotides, or from about 21 nucleotides to about 300 consecutive nucleotides.

As disclosed herein, 100% sequence identity between the dsRNA and the target gene is not required to practice the present invention. Preferably, the dsRNA of the invention comprises a 19-nucleotide portion which is substantially identical to at least 19 contiguous nucleotides of the target gene. While a dsRNA comprising a nucleotide sequence identical to a portion of the parasitic nematode target genes of the invention is preferred for inhibition, the invention can tolerate sequence variations that might be expected due to gene manipulation or synthesis, genetic mutation, strain polymorphism, or evolutionary divergence. Thus the dsRNAs of the invention also encompass dsRNAs comprising a mismatch with the target gene of at least 1, 2, or more nucleotides. For example, it is contemplated in the present invention that the 21mer dsRNA sequences exemplified in FIGS. 14a-14l may contain an addition, deletion or substitution of 1, 2, or more nucleotides, so long as the resulting sequence still interferes with the parasitic nematode target gene function.

Sequence identity between the dsRNAs of the invention and the parasitic nematode target genes may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and at least 19 contiguous nucleotides of the target gene is preferred.

When dsRNA of the invention has a length longer than about 21 nucleotides, for example from about 50 nucleotides to about 1000 nucleotides, it will be cleaved randomly to dsRNAs of about 21 nucleotides within the plant or parasitic nematode cell, the siRNAs. The cleavage of a longer dsRNA of the invention will yield a pool of 21 mer dsRNAs, derived from the longer dsRNA. This pool of 21 mer dsRNAs is also encompassed within the scope of the present invention, whether generated intracellularly within the plant or nematode or synthetically using known methods of oligonucleotide synthesis.

The siRNAs of the invention have sequences corresponding to fragments of 19-24 contiguous nucleotides across the entire sequence of the parasitic nematode target gene. For example, a pool of siRNA of the invention derived from the H. glycines target gene as set forth in SEQ ID NO:1, 3, 5, 7, 11, 13, 19, 21, 23, 25, 27, 104, 29, 39, 41, 43, 57, 59 or 61 may comprise a multiplicity of RNA molecules which are selected from the group consisting of oligonucleotides substantially identical to the 21mer nucleotides of SEQ ID NO: 1, 3, 5, 7, 11, 13, 19, 21, 23, 25, 104, 29, 39, 41, 43, 57, 59 or 61 found in FIGS. 14a-14l Similarly, the pool of siRNAs of the invention is also embodied in pools of 21mers of fragments and homologs of the H. glycines target genes as set forth in the table of FIGS. 14a-14l. One of skill in the art would recognize that the siRNA can have a mismatch with the target gene of at least 1, 2, or more nucleotides. Further, these mismatches are intended to be included in the present invention. For example, it is contemplated in the present invention that the 21mer dsRNA sequences exemplified in FIGS. 14a-14l may contain an addition, deletion or substitution of 1, 2, or more nucleotides and the resulting sequence still interferes with the nematode gene function. A pool of siRNA of the invention derived from the H. glycines target gene of SEQ ID NO: 1, 3, 5, 7, 11, 13, 19, 21, 23, 25, 27, 104, 29, 39, 41, 43, 57, 59 or 61 may also comprise any combination of the specific RNA molecules having any of the 21 contiguous nucleotide sequences derived from SEQ ID NO: 1, 3, 5, 7, 11, 13, 19, 21, 23, 25, 104, 29, 39, 41, 43, 57, 59 or 61 set forth in FIGS. 14a-14l. Further, as multiple specialized Dicers in plants generate siRNAs typically ranging in size from 19nt to 24nt (See Henderson et al., 2006. Nature Genetics 38:721-725.), the siRNAs of the present invention can may range from 19 contiguous nucleotide sequences to about 24 contiguous nucleotide sequences. Similarly, a pool of siRNA of the invention may comprise a multiplicity of RNA molecules having any 19, 20, 21, 22, 23, or 24 contiguous nucleotide sequences derived from SEQ ID NO: 1, 3, 5, 7, 11, 13, 19, 21, 23, 25, 27, 104, 29, 39, 41, 43, 57, 59 or 61. Alternatively, the pool of siRNA of the invention may comprise a multiplicity of RNA molecules having a combination of any 19, 20, 21, 22, 23,and/or 24 contiguous nucleotide sequences derived from SEQ ID NO: 1, 3, 5, 7, 11, 13, 19, 21, 23, 25, 27, 104, 29, 39, 41, 43, 57, 59 or 61.

The dsRNA of the invention may optionally comprise a single stranded overhang at either or both ends. Preferably, the single stranded overhang comprises at least two nucleotides at the 3′ end of each strand of the dsRNA molecule. Synthetic siRNAs may comprise 2′-deoxythymidine (TT) or ribo-uridine (UU) in the two-nucleotide overhanging portion. The double-stranded structure may be formed by a single self-complementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

In another embodiment, the invention provides an isolated recombinant expression vector comprising a nucleic acid encoding a dsRNA molecule as described above, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Gemini virus), which serve equivalent functions.

The recombinant expression vectors of the invention comprise a nucleic acid of the invention in a form suitable for expression of the nucleic acid in a host plant cell, which means that the recombinant expression vector includes one or more regulatory sequences, e.g. promoters, selected on the basis of the host plant cells to be used for expression, which is operatively linked to the nucleic acid sequence to be expressed. With respect to a recombinant expression vector, the terms “operatively linked” and “in operative association” are interchangeable and are intended to mean that the nucleotide sequence of interest is linked to the regulatory sequence(s) in a manner which allows for expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, and the like. The expression vectors of the invention can be introduced into plant host cells to thereby produce dsRNA molecules of the invention encoded by nucleic acids as described herein.

In accordance with the invention, the recombinant expression vector comprises a regulatory sequence operatively linked to a nucleotide sequence that is a template for one or both strands of the dsRNA molecules of the invention. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 base or more pairs, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M.W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter, or a nematode inducible promoter. More preferably the nematode inducible promoter is or a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its un-induced state. A promoter is cell-, tissue- or organ-specific, if its activity, measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leaves, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sepl promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode, in particular cells of, or close by nematode feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Geminil9, and AtHMG1 have been shown to be induced by nematodes (for a review of nematode-inducible promoters, see Ann. Rev. Phytopathol. (2002) 40:191-219; see also U.S. Pat. No. 6,593,513).

Preferred nematode-inducible promoters are disclosed in commonly-assigned copending applications PCT/EP2007/, PCT/EP2007/, PCT/EP2007/, and PCT/EP2008/. Most preferably, the nematode-inducible promoters having SEQ ID NOs:69, 70, and 71 are employed in the expression vector of the invention.

Methods for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adhl promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the 1pt2 or 1ptl-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15kD zein promoter, the 22kD zein promoter, the 27kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

In accordance with the present invention, the expression cassette comprises an expression control sequence operatively linked to a nucleotide sequence that is a template for one or both strands of the dsRNA. The dsRNA template comprises (a) a first stand having a sequence substantially identical to from 19 to about 400-500, or up to the full length, consecutive nucleotides of SEQ ID NO:1, 3, 5, 7, 9, 11, 13, 15, 19, 21, 23, 25, 27, 104, 29, 35, 37, 39, 41, 43, 45, 47, 49, 51, 57, 59, 61, 63, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 106, or 107 and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 base pairs, and wherein after transcription, the RNA transcript folds on itself to form a hairpin.

In another embodiment, the vector contains a bidirectional promoter, driving expression of two nucleic acid molecules, whereby one nucleic acid molecule codes for the sequence substantially identical to a portion of a parasitic nematode innexin-like, pas-1, tcp-1, snurportin-1 like, pol delta S, prs-4, rtp-1 or rpn-5 target gene and the other nucleic acid molecule codes for a second sequence being substantially complementary to the first strand and capable of forming a dsRNA, when both sequences are transcribed. A bidirectional promoter is a promoter capable of mediating expression in two directions.

In another embodiment, the vector contains two promoters, one mediating transcription of the sequence substantially identical to a portion of a parasitic nematode innexin-like, pas-1, tcp-1, snurportin-1 like, pol delta S, prs-4, rtp-1 or rpn-5 target gene and another promoter mediating transcription of a second sequence being substantially complementary to the first strand and capable of forming a dsRNA, when both sequences are transcribed. The second promoter might be a different promoter.

A different promoter means a promoter having a different activity in regard to cell or tissue specificity, or showing expression on different inducers for example, pathogens, abiotic stress or chemicals. For example, one promoter might by constitutive or tissue specific and another might be tissue specific or inducible by pathogens. In one embodiment one promoter mediates the transcription of one nucleic acid molecule suitable for over expression of an innexin-like, pas-1, tcp-1, snurportin-1 like, pol delta S, prs-4, rtp-1 or rpn-5 gene, while another promoter mediates tissue- or cell-specific transcription or pathogen inducible expression of the complementary nucleic acid.

The invention is also embodied in a transgenic plant capable of expressing the dsRNA of the invention and thereby inhibiting the innexin-like, pas-1, tcp-1, snurportin-1 like, pol delta S, prs-4, rtp-1 and rpn-5 genes in parasitic nematodes. The plant or transgenic plant may be any plant, such like, but not limited to trees, cut flowers, ornamentals, vegetables or crop plants. The plant may be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium, or the plant may be selected from a genus selected from the group consisting of Arabidopsis, Medicago, Lycopersicon, Brassica, Cucumis, Solanum, Juglans, Gossypium, Malus, Vitis, Antirrhinum, Brachipodium, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panicum, Pennisetum, Ranunculus, Senecio, Salpiglossis, Browaalia, Phaseolus, Avena, and Allium. In one embodiment the plant is a monocotyledonous plant or a dicotyledonous plant.

Preferably the plant is a crop plant. Crop plants are all plants, used in agriculture. Accordingly in one embodiment the plant is a monocotyledonous plant, preferably a plant of the family Poaceae, Musaceae, Liliaceae or Bromeliaceae, preferably of the family Poaceae. Accordingly, in yet another embodiment the plant is a Poaceae plant of the genus Zea, Triticum, Oryza, Hordeum. Secale, Avena, Saccharum, Sorghum, Pennisetum, Setaria, Panicum, Eleusine, Miscanthus, Brachypodium, Festuca or Lolium. When the plant is of the genus Zea, the preferred species is Z. mays. When the plant is of the genus Triticum, the preferred species is T. aestivum, T. speltae or T. durum. When the plant is of the genus Oryza, the preferred species is O. sativa. When the plant is of the genus Hordeum, the preferred species is H. vulgare. When the plant is of the genus Secale, the preferred species S. cereale. When the plant is of the genus Avena, the preferred species is A. sativa. When the plant is of the genus Saccarum, the preferred species is S. officinarum. When the plant is of the genus Sorghum, the preferred species is S. vulgare, S. bicolor or S. sudanense. When the plant is of the genus Pennisetum, the preferred species is P. glaucum. When the plant is of the genus Setaria, the preferred species is S. italica. When the plant is of the genus Panicum, the preferred species is P. miliaceum or P. virgatum. When the plant is of the genus Eleusine, the preferred species is E. coracana. When the plant is of the genus Miscanthus, the preferred species is M. sinensis. When the plant is a plant of the genus Festuca, the preferred species is F. arundinaria, F. rubra or F. pratensis. When the plant is of the genus Lolium, the preferred species is L. perenne or L. multiflorum. Alternatively, the plant may be Triticosecale.

Alternatively, in one embodiment the plant is a dicotyledonous plant, preferably a plant of the family Fabaceae, Solanaceae, Brassicaceae, Chenopodiaceae, Asteraceae, Malvaceae, Linacea, Euphorbiaceae, Convolvulaceae Rosaceae, Cucurbitaceae, Theaceae, Rubiaceae, Sterculiaceae or Citrus. In one embodiment the plant is a plant of the family Fabaceae, Solanaceae or Brassicaceae. Accordingly, in one embodiment the plant is of the family Fabaceae, preferably of the genus Glycine, Pisum, Arachis, Cicer, Vicia, Phaseolus, Lupinus, Medicago or Lens. Preferred species of the family Fabaceae are M. truncatula, M, sativa, G. max, P. sativum, A. hypogea, C. arietinum, V. faba, P. vulgaris, Lupinus albus, Lupinus luteus, Lupinus angustifolius or Lens culinaris. More preferred are the species G. max A. hypogea and M. sativa. Most preferred is the species G. max. When the plant is of the family Solanaceae, the preferred genus is Solanum, Lycopersicon, Nicotiana or Capsicum. Preferred species of the family Solanaceae are S. tuberosum, L. esculentum (also known as Solanum lycopersicon), N. tabaccum or C. chinense. More preferred is S. tuberosum. Accordingly, in one embodiment the plant is of the family Brassicaceae, preferably of the genus Brassica or Raphanus. Preferred species of the family Brassicaceae are the species B. napus, B. oleracea, B. juncea or B. rapa. More preferred is the species B. napus. When the plant is of the family Chenopodiaceae, the preferred genus is Beta and the preferred species is the B. vulgaris. When the plant is of the family Asteraceae, the preferred genus is Helianthus and the preferred species is H. annuus. When the plant is of the family Malvaceae, the preferred genus is Gossypium or Abelmoschus. When the genus is Gossypium, the preferred species is G. hirsutum or G. barbadense and the most preferred species is G. hirsutum. A preferred species of the genus Abelmoschus is the species A. esculentus. When the plant is of the family Linacea, the preferred genus is Linum and the preferred species is L. usitatissimum. When the plant is of the family Euphorbiaceae, the preferred genus is Manihot, Jatropa or Rhizinus and the preferred species are M. esculenta, J. curcas or R. comunis. When the plant is of the family Convolvulaceae, the preferred genus is Ipomea and the preferred species is I. batatas. When the plant is of the family Rosaceae, the preferred genus is Rosa, Malus, Pyrus, Prunus, Rubus, Ribes, Vaccinium or Fragaria and the preferred species is the hybrid Fragaria x ananassa. When the plant is of the family Cucurbitaceae, the preferred genus is Cucumis, Citrullus or Cucurbita and the preferred species is Cucumis sativus, Citrullus lanatus or Cucurbita pepo. When the plant is of the family Theaceae, the preferred genus is Camellia and the preferred species is C. sinensis. When the plant is of the family Rubiaceae, the preferred genus is Coffea and the preferred species is C. arabica or C. canephora. When the plant is of the family Sterculiaceae, the preferred genus is Theobroma and the preferred species is T. cacao. When the plant is of the genus Citrus, the preferred species is C. sinensis, C. limon, C. reticulata, C. maxima and hybrids of Citrus species, or the like. In a preferred embodiment of the invention, the plant is a soybean, a potato or a corn plant

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used. Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm ME et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, Ml3mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The Agrobacterium-mediated transformation is best suited to dicotyledonous plants but has also been adapted to monocotyledonous plants. The transformation of plants by Agrobacteria is described in, for example, White FF, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225. Transformation may result in transient or stable transformation and expression. Although a nucleotide sequence of the present invention can be inserted into any plant and plant cell falling within these broad classes, it is particularly useful in crop plant cells.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In accordance with this embodiment, the transgenic plant of the invention is produced by a method comprising the steps of providing a parasitic nematode innexin-like, pas-1, tcp-1, snurportin-1 like, pol delta S, prs-4, rtp-1 or rpn-5 target gene, preparing an expression cassette having a first region that is substantially identical to a portion of the selected innexin-like, pas-1, tcp-1, snurportin-1 like, pol delta S, prs-4, rtp-1 or rpn-5 gene and a second region which is complementary to the first region, transforming the expression cassette into a plant, and selecting progeny of the transformed plant which express the dsRNA construct of the invention.

As increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells. Nematodes inducing giant or syncytial cells are found in the families Longidoridae, Trichodoridae, Heterodidae, Meloidogynidae, Pratylenchidae or Tylenchulidae. In particular in the families Heterodidae and Meloidogynidae.

Accordingly, parasitic nematodes targeted by the present invention belong to one or more genus selected from the group ofNaccobus, Cactodera, Dolichodera, Globodera, Heterodera, Punctodera, Longidorus or Meloidogyne. In a preferred embodiment the parasitic nematodes belong to one or more genus selected from the group of Naccobus, Cactodera, Dolichodera, Globodera, Heterodera, Punctodera or Meloidogyne. In a more preferred embodiment the parasitic nematodes belong to one or more genus selected from the group of Globodera, Heterodera, or Meloidogyne. In an even more preferred embodiment the parasitic nematodes belong to one or both genera selected from the group of Globodera or Heterodera. In another embodiment the parasitic nematodes belong to the genus Meloidogyne.

When the parasitic nematodes are of the genus Globodera, the species are preferably from the group consisting of G. achilleae, G. artemisiae, G. hypolysi, G. mexicana, G. millefolii, G. mall, G. pallida, G. rostochiensis, G. tabacum, and G. virginiae. In another preferred embodiment the parasitic Globodera nematodes includes at least one of the species G. pallida, G. tabacum, or G. rostochiensis. When the parasitic nematodes are of the genus Heterodera, the species may be preferably from the group consisting of H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. In another preferred embodiment the parasitic Heterodera nematodes include at least one of the species H. glycines, H. avenae, H. cajani, H. gottingiana, H. trifolii, H. zeae or H. schachtii. In a more preferred embodiment the parasitic nematodes includes at least one of the species H. glycines or H. schachtii. In a most preferred embodiment the parasitic nematode is the species H. glycines. When the parasitic nematodes are of the genus Meloidogyne, the parasitic nematode may be selected from the group consisting of M. acronea, M arabica, M. arenaria, M artiellia, M brevicauda, M. camelliae, M. chitwoodi, M. cofeicola, M. esigua, M graminicola, M hapla, M. incognita, M. indica, M. inornata, M. javanica, M lini, M. mali, M microcephala, M microtyla, M. naasi, M. salasi and M. thamesi. In a preferred embodiment the parasitic nematodes includes at least one of the species M. javanica, M incognita, M. hapla, M. arenaria or M. chitwoodi.

The following examples are not intended to limit the scope of the claims to the invention, but are rather intended to be exemplary of certain embodiments. Any variations in the exemplified methods that occur to the skilled artisan are intended to fall within the scope of the present invention.

### EXAMPLE 1

### Identification and Isolation of H. Glycines Rnai Target Genes

Using total RNA isolated from SCN J2 stage, RT-PCR was used to isolate cDNA fragments approximately 400-500 by in length that were used to construct the binary vectors discussed in Example 2. The PCR products were cloned into TOPO pCR2.1 vector (Invitrogen, Carlsbad, Calif.) and inserts were confirmed by sequencing. Gene fragments for all eight target genes were isolated using this method.

In order to obtain full-length cDNA for H. glycines target genes, a RT-PCR method, based on highly conserved spliced leader sequence (SL1) present in many nematode species, was used. The reactions were conducted using Superscript One-Step kit (Invitrogen, Carlsbad, Calif., catalog no. 10928-034) and a primer set. The forward primer consisted of a 22-mer SL1 sequence, and reverse primers were gene specific and located in the previously cloned cDNA region. PCR products were cloned into a pCR4-TOPO vector (Invitrogen, Carlsbad, Calif.) and sequenced.

3′cDNA ends were amplified using the GeneRacer Kit (Invitrogen, Carlsbad, Calif., catalog No. L1500-01). The first-strand cDNAs were generated through reverse transcription using total RNA and the GeneRacer Oligo dT Primer. The 3′ RACE PCR was performed with the GeneRacer 3′ Primer and a gene-specific forward primer. Nested PCR reactions were subsequently conducted using the GeneRacer 3′ Nested Primer and a gene-specific forward primer. PCR products were cloned into a pCR4-TOPO (Invitrogen, Carlsbad, Calif.) and sequenced.

The full length sequences for each of the eight SCN target genes were assembled into cDNAs corresponding to the eight gene targets, designated as SEQ ID NO:1, SEQ ID NO:5, SEQ ID NO:11, SEQ ID NO:19, SEQ ID NO:23, SEQ ID NO:39, SEQ ID NO:57 and SEQ ID NO:104.

### EXAMPLE 2

### Binary Vector Construction for Soybean Transformation.

In order to evaluate whether the SCN targets are effective in vivo, cDNA fragments for eight SCN target genes were used to make binary vectors. The vectors consist of an antisense fragment of the target (e.g. H. glycines tcp-1), a spacer fragment, a sense fragment of target (e.g. H. glycines tcp-1) and a vector backbone. In this vector, dsRNA for the target gene was expressed under a constitutive Super Promoter (see U.S. Pat. No. 5955,646, incorporated herein by reference). The selection marker for transformation was a mutated acetohydroxyacid synthase (AHAS) gene from Arabidopsis thaliana conferring resistance to the herbicide ARSENAL (Imazapyr, BASF Corporation, Florham Park, N.J.). The expression of the mutated AHAS was driven by a ubiquitin promoter.

A gene fragment corresponding to SEQ ID NO:3 was used to construct the binary vector RTP1030. A gene fragment corresponding to SEQ ID NO:7 was used to construct the binary vector RTP1095. A gene fragment corresponding to SEQ ID NO:13 was used to construct the binary vector RSA131. A gene fragment corresponding to SEQ ID NO:21 was used to construct the binary vector RSA123. A gene fragment corresponding to SEQ ID NO:25 was used to construct the binary vector RCB987. A gene fragment corresponding to SEQ ID NO:29 was used to construct the binary vector RTP1169. A gene fragment corresponding to SEQ ID NO:41 was used to construct the binary vector RSA012. A gene fragment corresponding to SEQ ID NO:59 was used to construct the binary vector RTP1269.

### EXAMPLE 3

### Bioassay of Dsrna Targeted to H. Glycines Target Genes

A rooted explant assay was employed to demonstrate dsRNA expression and the resulting nematode resistance. Details of this assay can be found in co-pending application U.S. Ser. No. 12/001,234, the contents of which are incorporated herein by reference. The binary vectors RTP1030, RCB987, RSA131, RTP1095, RSA123, RSA012, RTP1169, RTP1269 described in Example 2 were transfected into the disarmed A. rhizogenes strain K599, and soybean cotyledons containing the proximal end from its connection with the seedlings were used as the explant for transformation. Two to three weeks after inoculation and root induction in accordance with the method of U.S. Ser. No. 12/001,234, transformed roots were formed on the cut ends of the explants. Soybean roots were excised from the rooted explants, subcultured, and one to five days after subculturing the roots were inoculated with surface sterilized SCN J2 juveniles in multi-well plates for the gene of interest construct assay. As controls, soybean cultivar Williams 82 control vector and Jack control vector roots were used. Four weeks after nematode inoculation, the cysts in each well were counted. Bioassay results for constructs RTP1030, RCB987, RSA131, RTP1095, RSA123, RSA012, RTP1169, and RTP1269 resulted in multiple lines with reduced cyst count showing a general trend of reduced cyst count over many of the lines tested.

### EXAMPLE 4

### Description of Homologs and Dna Sequence Motifs

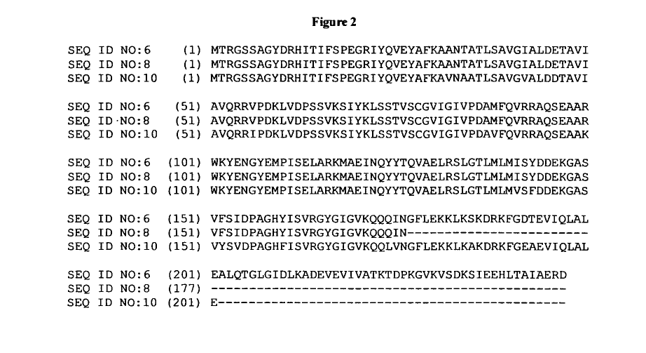

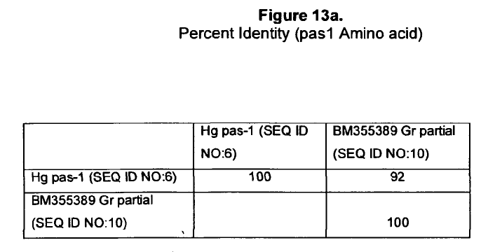

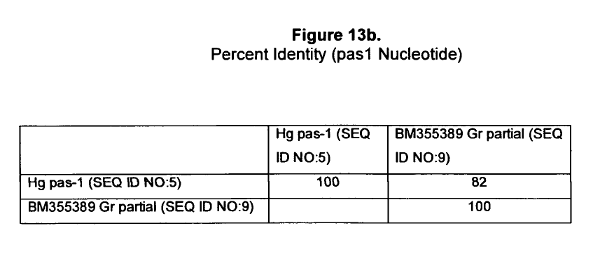

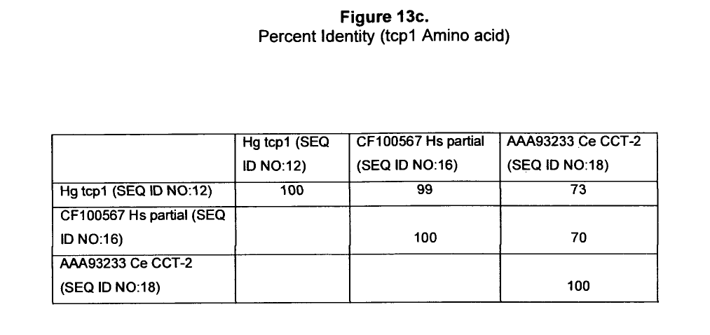

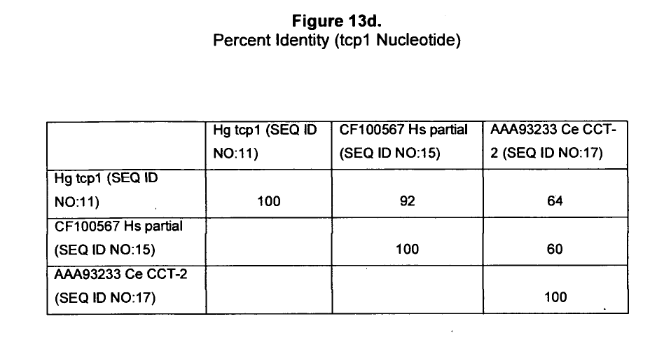

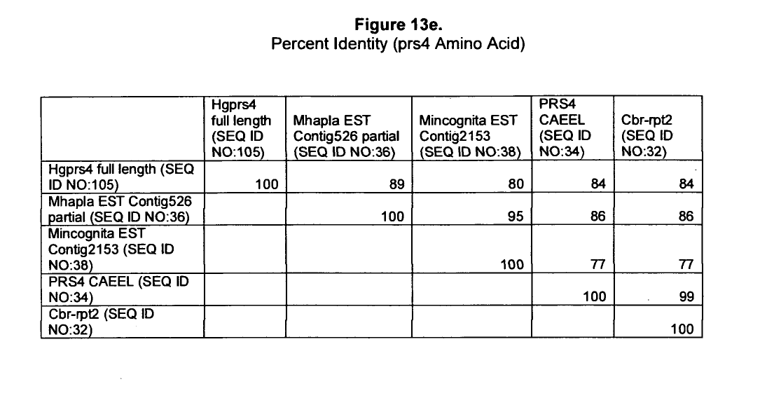

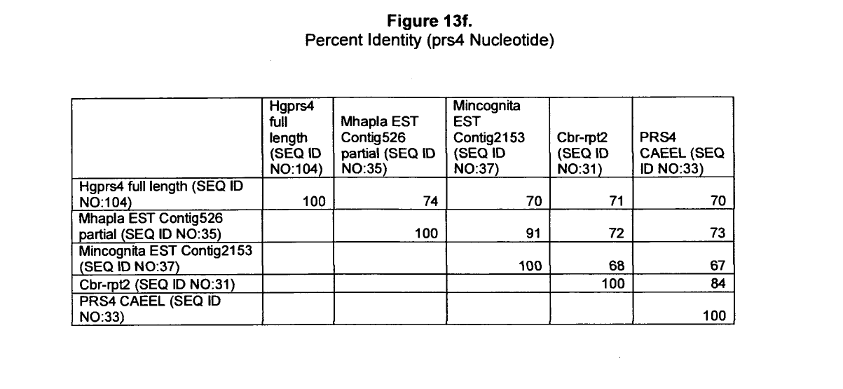

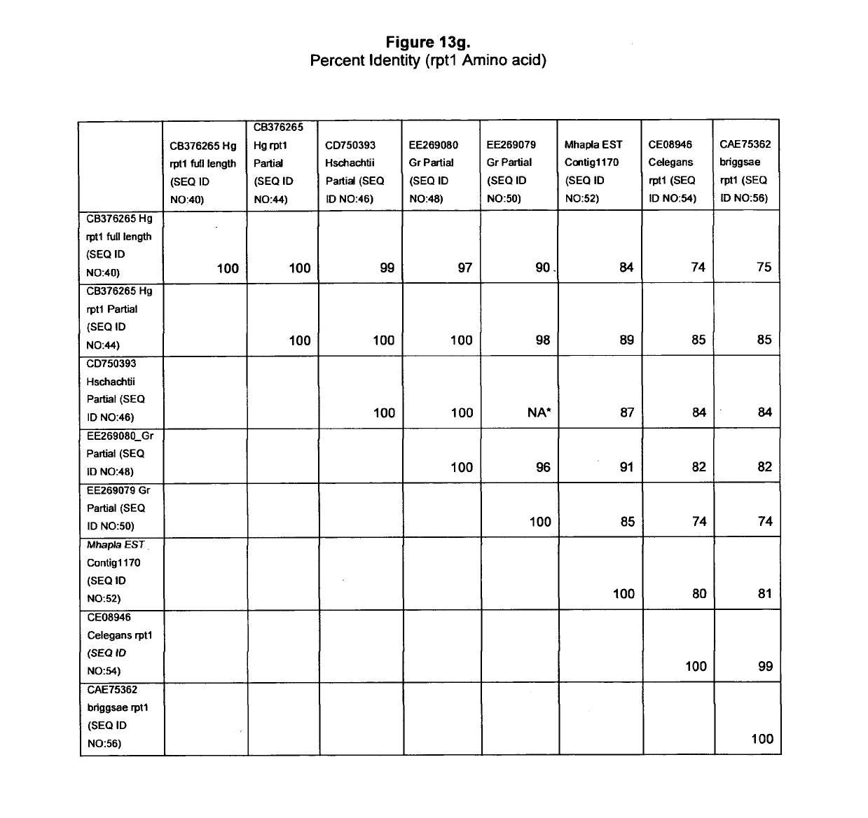

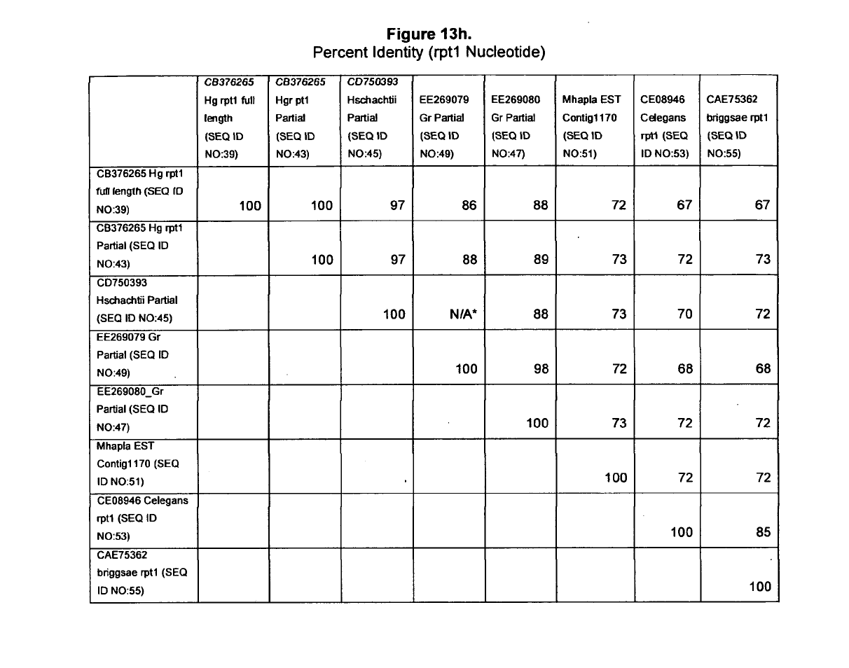

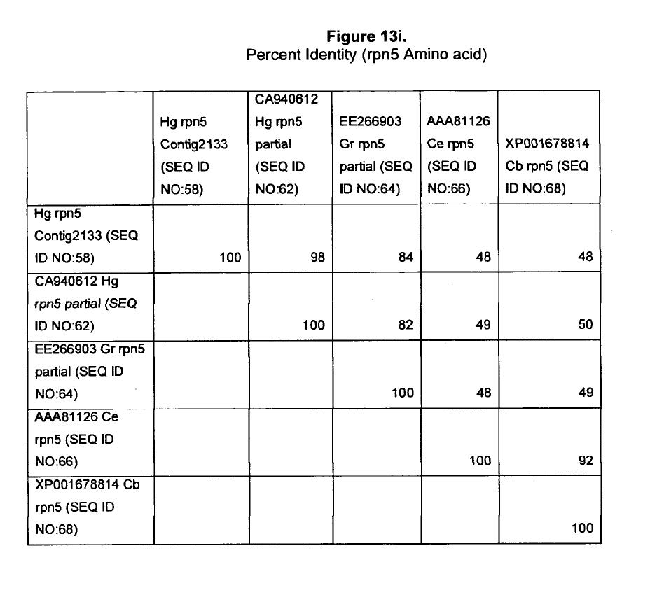

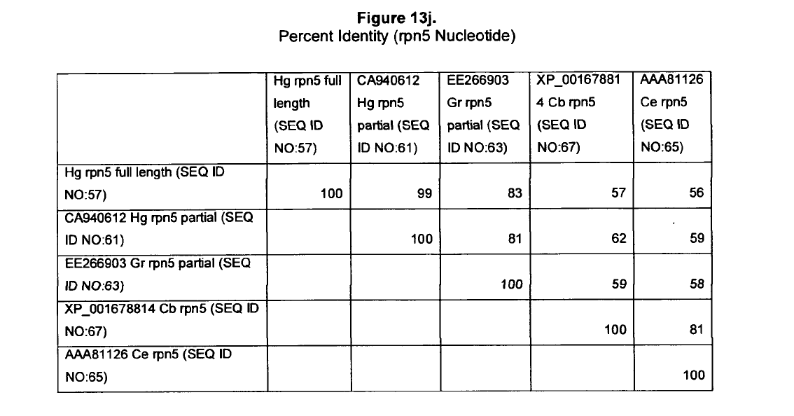

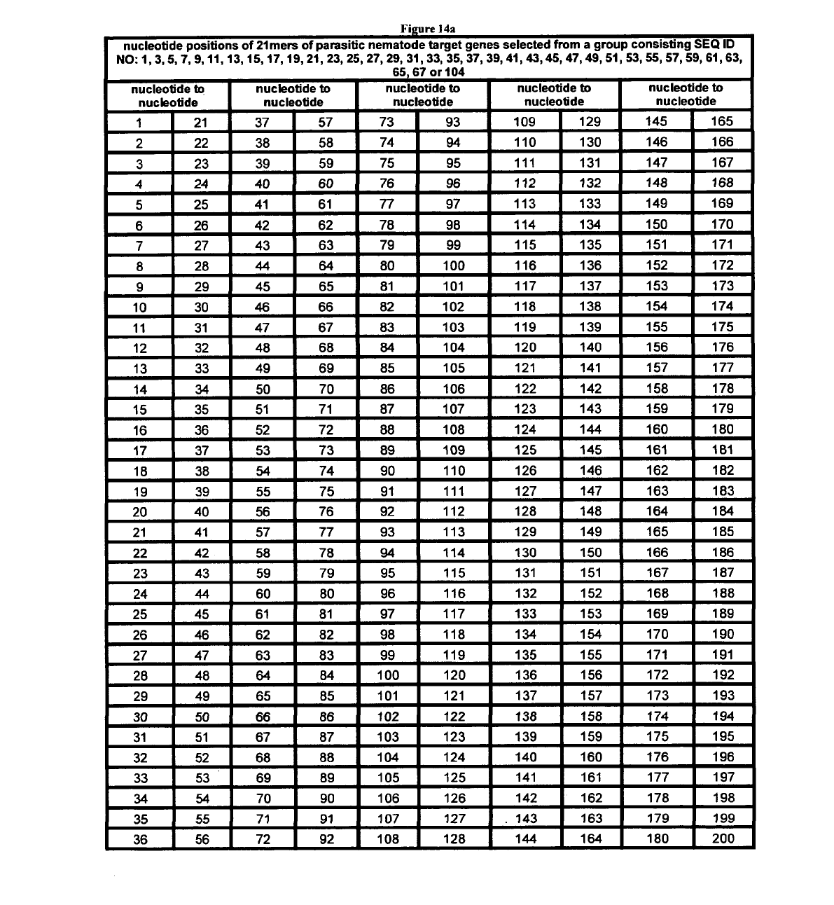

As disclosed in Example 3, the construct RTP1095 results in the expression of a double stranded RNA molecule that targets SEQ ID NO:5 and results in reduced cyst count when operably linked to a constitutive promoter and expressed in soybean roots. As disclosed in Example 1, the putative full length transcript sequence of the gene described by SEQ ID NO:5 contains an open reading frame with the amino acid sequence disclosed as SEQ ID NO:6. The amino acid sequence described by SEQ ID NO:6 was used to identify homologous genes. A sample gene with DNA and amino acid sequences homologous to SEQ ID NO:5 and SEQ ID NO:6, respectively, were identified and are described by SEQ ID NO:9 and SEQ ID NO:10. The amino acid alignment of the identified homolog to SEQ ID NO:6 is shown in FIG. 2. A matrix table showing the amino acid percent identity of the identified homolog and SEQ ID NO:6 to each other is shown in FIG. 13a. The DNA sequence alignment of the identified homolog SEQ ID NO:9 to SEQ ID NO:5 is shown in FIG. 7a-b. Regions of high homology alignment over 21 nucleotides or more are marked as Motif A through Motif G in FIG. 7a-b. The motif sequences corresponding to Motif A through Motif G are described by SEQ ID NOs 72-78. A matrix table showing the DNA sequence percent identity of SEQ ID NO:5 and the identified homolog SEQ ID NO:9 to each other is shown in FIG. 13b.

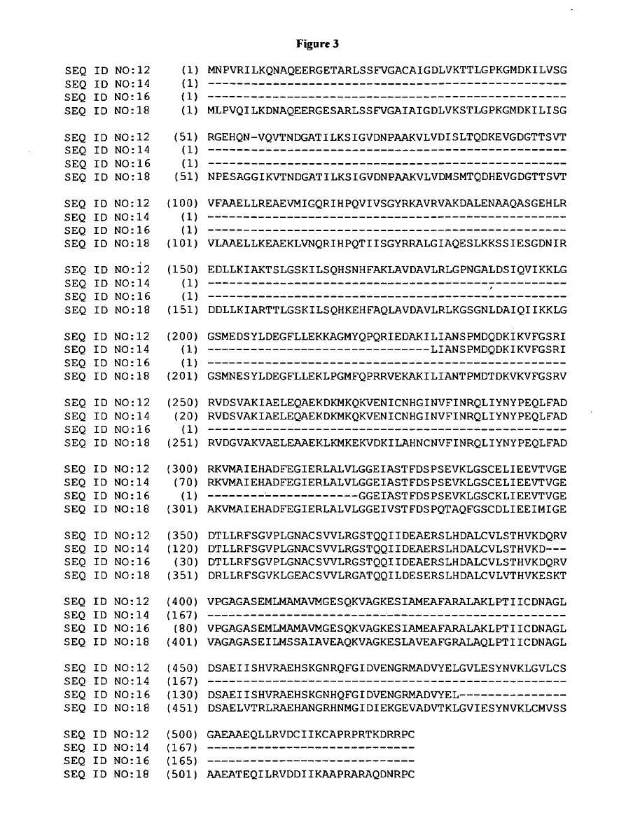

As disclosed in Example 3, the construct RSA131 results in the expression of a double stranded RNA molecule that targets SEQ ID NO:11 and results in reduced cyst count when operably linked to a constitutive promoter and expressed in soybean roots. As disclosed in Example 1, the putative full length transcript sequence of the gene described by SEQ ID NO:11 contains an open reading frame with the amino acid sequence disclosed as SEQ ID NO:12. The amino acid sequence described by SEQ ID NO:12 was used to identify homologous genes. Plant parasitic nematode genes with DNA and amino acid sequences homologous to SEQ ID NO:11 and SEQ ID NO:12, respectively, were identified and are described by SEQ ID NOs 15-18. The amino acid alignment of the identified homolog to SEQ ID NO:12 is shown in FIG. 3. A matrix table showing the amino acid percent identity of the identified homolog and SEQ ID NO:6 to each other is shown in FIG. 13c. The DNA sequence alignment of the identified homolog described by SEQ ID NO:15 to SEQ ID NO:11 is shown in FIG. 8a-c. Regions of high homology alignment over 21 nucleotides or more are marked as Motif A through Motif G in FIG. 8a-c. The motif sequences corresponding to Motif A through Motif F are described by SEQ ID NOs 86-91. A matrix table showing the DNA sequence percent identity of SEQ ID NO:11 and the identified homologs to each other is shown in FIG. 13d.

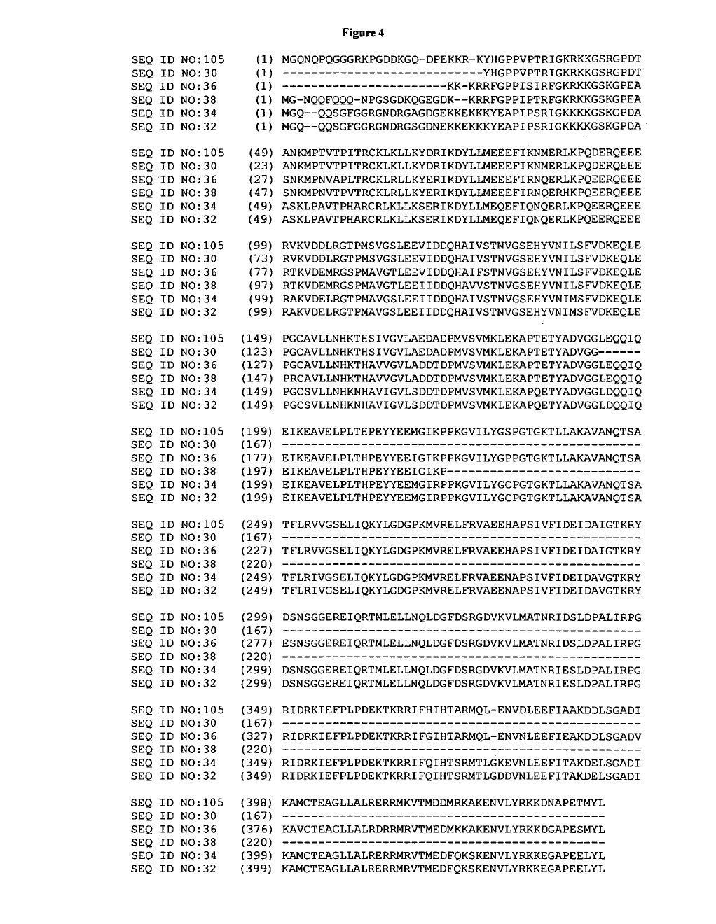

As disclosed in Example 3, the construct RTP1169 results in the expression of a double stranded RNA molecule that targets SEQ ID NO:27 and SEQ ID NO:104 and results in reduced cyst count when operably linked to a constitutive promoter and expressed in soybean roots. The sequence described by SEQ ID NO:27 is a partial DNA sequence and does not represent the full length coding sequence of the associated gene. The amino acid sequence of this partial DNA sequence is represented by SEQ ID NO:28. The putative full length sequence of the associated gene described by SEQ ID NO:27 was derived using 5′ and 3′ RACE and is described by SEQ ID NO:104. The amino acid sequence of the putative full length sequence is described by SEQ ID NO:105. The amino acid sequence described by SEQ ID NO:105 was used to identify homologous genes. Plant parasitic nematode genes with DNA and amino acid sequences homologous to SEQ ID NO:104 and SEQ ID NO:105, respectively, were identified and are described by SEQ ID NOs 31-38. The amino acid alignment of the identified homologs to SEQ ID NO:105 is shown in FIG. 4. A matrix table showing the amino acid percent identity of the identified homolog and SEQ ID NO:105 to each other is shown in FIG. 13e. The DNA sequence alignment of the identified homologs to SEQ ID NO:104 is shown in FIG. 9a-b. Regions of high homology alignment over 21 nucleotides or more are marked as Motif A through Motif D in FIG. 9a-b. The motif sequences corresponding to Motif A and Motif B are described by SEQ ID NOs 92, 93, 106, and 107. A matrix table showing the DNA sequence percent identity of SEQ ID NO:104 and the identified homologs to each other is shown in FIG. 13f.

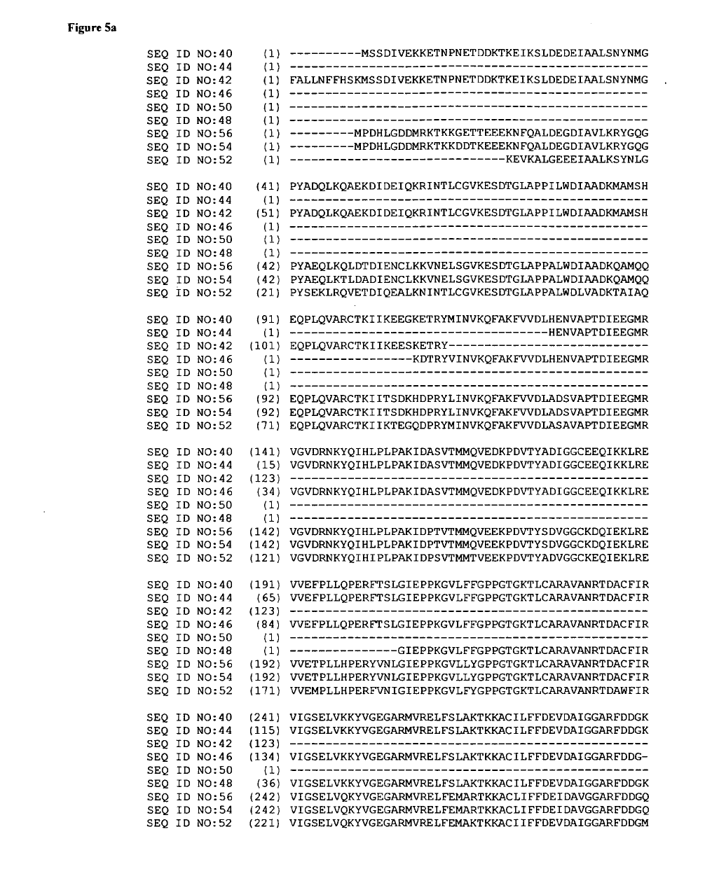

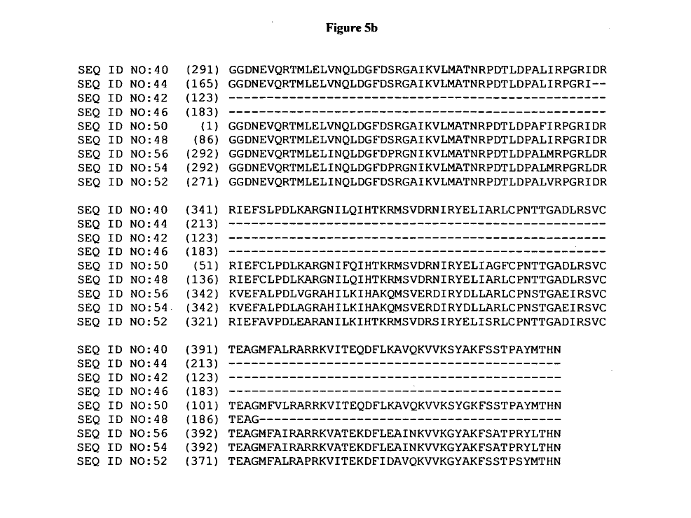

As disclosed in Example 3, the construct RSA012 results in the expression of a double stranded RNA molecule that targets SEQ ID NO:39 and results in reduced cyst count when operably linked to a constitutive promoter and expressed in soybean roots. As disclosed in Example 1, the putative full length transcript sequence of the gene described by SEQ ID NO:39 contains an open reading frame with the amino acid sequence disclosed as SEQ ID NO:40. The amino acid sequence described by SEQ ID NO:40 was used to identify homologous genes. Plant parasitic nematode genes with DNA and amino acid sequences homologous to SEQ ID NO:39 and SEQ ID NO:40, respectively, were identified and are described by SEQ ID NOs 43-56. The amino acid alignment of the identified homologs to SEQ ID NO:40 is shown in FIG. 5a-b. A matrix table showing the amino acid percent identity of the identified homolog and SEQ ID NO:40 to each other is shown in FIG. 13g. The DNA sequence alignment of the identified homologs of SEQ ID NO:39 are shown in FIG. 10a-e. Regions of high homology alignment over 21 nucleotides or more are marked as Motif A through Motif J in FIG. 10a-e. The motif sequences corresponding to Motif A through Motif J are described by SEQ ID NOs 94-103. A matrix table showing the DNA sequence percent identity of SEQ ID NO:39 and the identified homologs to each other is shown in FIG. 13h.

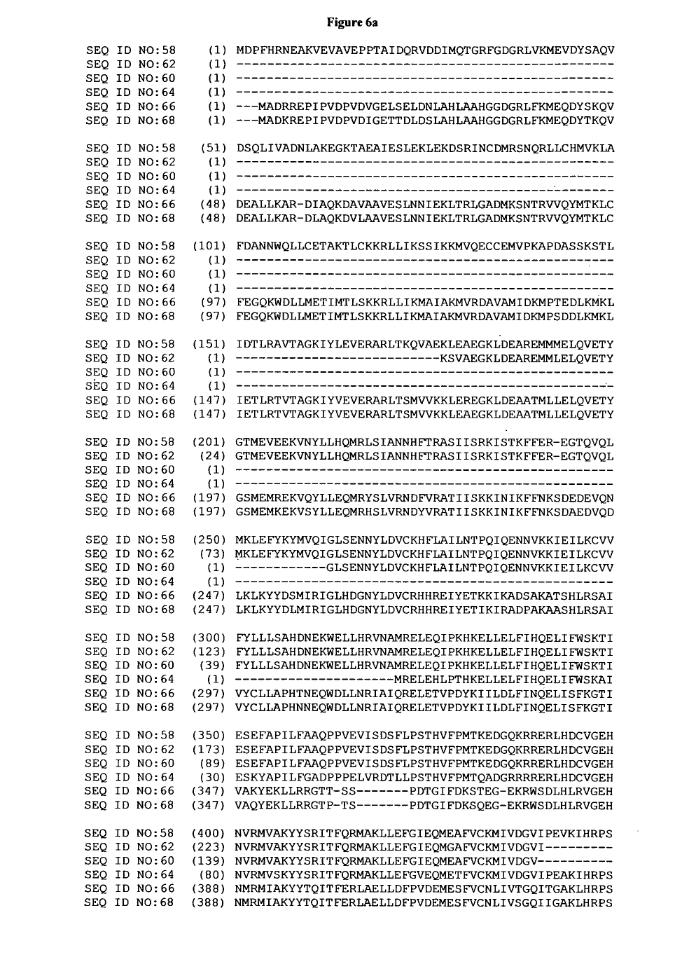

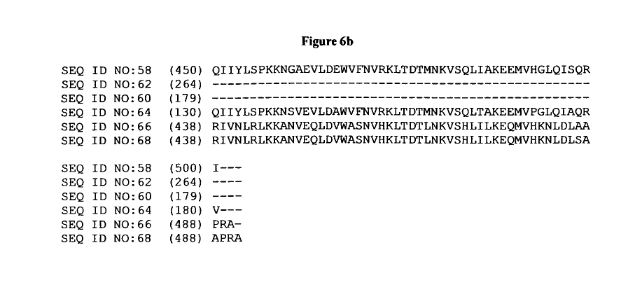

As disclosed in Example 3, the construct RTP1269 results in the expression of a double stranded RNA molecule that targets SEQ ID NO:57 and results in reduced cyst count when operably linked to a constitutive promoter and expressed in soybean roots. As disclosed in Example 1, the putative full length transcript sequence of the gene described by SEQ ID NO:57 contains an open reading frame with the amino acid sequence disclosed as SEQ ID NO:58. The amino acid sequence described by SEQ ID NO:58 was used to identify homologous genes. Plant parasitic nematode genes with DNA and amino acid sequences homologous to SEQ ID NO:57 and SEQ ID NO:58, respectively, were identified and are described by SEQ ID NOs 61-68. The amino acid alignment of the identified homolog to SEQ ID NO:58 is shown in FIG. 6a-b. A matrix table showing the amino acid percent identity of the identified homologs and SEQ ID NO:58 to each other is shown in FIG. 13i. The DNA sequence alignment of the identified homologs to SEQ ID NO:57 are shown in FIG. 11a-b. Regions of high homology alignment over 21 nucleotides or more are marked as Motif A through Motif G in FIG. 11a-b. The motif sequences corresponding to Motif A through Motif G are described by SEQ ID NOs 79-85. A matrix table showing the DNA sequence percent identity of SEQ ID NO:57 and the identified homologs to each other is shown in FIG. 13j.

1. A double stranded RNA molecule comprising (a) a first strand having a sequence substantially identical to from 19 to about 400 or 500 consecutive nucleotides of a plant parasitic nematode target gene selected from a group consisting of a parasitic nematode innexin-like gene, a parasitic nematode gene encoding a polymerase delta small subunit (pol delta 5), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

2. The double stranded RNA of claim 1, wherein the first strand has a sequence substantially identical to from 19 to about 400 or 500 consecutive nucleotides of a target gene having a sequence selected from the group of NO:1, 3, 5, 7, 9, 11, 13, 15, 19, 21, 23, 25, 27, 104, 29, 35, 37, 39, 41, 43, 45, 47, 49, 51, 57, 59, 61, 63, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 106, or 107 and (b) a second strand having a sequence substantially complementary to the first strand.

3. A pool of double stranded RNA molecules comprising a multiplicity of short interfering RNA molecules each comprising a double stranded region having a length of 19 to 24 nucleotides, wherein said RNA molecules are derived from a polynucleotide selected from the group consisting of a parasitic nematode innexin-like gene, a parasitic nematode gene encoding a polymerase delta small subunit (pol delta 5), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

4. The pool of double stranded RNA molecules of claim 3, wherein the RNA molecules are derived from the polynucleotide selected from the group consisting of (a) a polynucleotide having a sequence as set forth in SEQ ID NO:1 and 3; (b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.

5. A transgenic plant resistant to parasitic nematode infection, the plant comprising a nucleic acid construct that encodes a dsRNA capable of specifically decreasing expression of a parasitic nematode innexin-like gene, a parasitic nematode gene encoding a polymerase delta small subunit (pol delta 5), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), or a parasitic nematode gene homologous to a C. elegans rpn-5 gene.

6. The transgenic plant of claim 5, wherein the dsRNA targets a polynucleotide selected from the group consisting of (a) a polynucleotide having a sequence as set forth in SEQ ID NO:1 and 3;
(b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.

7. A transgenic plant capable of expressing a pool of dsRNA molecules, wherein each dsRNA molecule comprises a double stranded region having a length of 19-24 nucleotides and wherein the RNA molecules are derived from polynucleotides substantially identical to a portion of a parasitic nematode target gene selected from the group consisting of a parasitic nematode innexin-like gene, a parasitic nematode gene encoding a polymerase delta small subunit (pol delta 5), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

8. The transgenic plant of claim 7, wherein the pool of dsRNA targets a polynucleotide selected from the group consisting of (a) a polynucleotide having a sequence as set forth in SEQ ID NO:1 and 3; (b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.

9. A method of making a transgenic plant capable of expressing a dsRNA that is substantially identical to a target gene in a parasitic nematode, said method comprising the steps of: (a) selecting a target gene from the group consisting of a parasitic nematode innexin-like gene, a parasitic nematode gene encoding a polymerase delta small subunit (pol delta 5), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene; (b) preparing a nucleic acid sequence comprising a region that is substantially identical to a portion of the selected target gene, wherein the nucleic acid is able to form a double-stranded transcript once expressed in the plant; (c) transforming a recipient plant with said nucleic acid; (d) producing one or more transgenic offspring of said recipient plant; and (e) selecting the offspring for nematode resistance.

10. The method of claim 9, wherein the dsRNA targets a polynucleotide selected from the group consisting of (a) a polynucleotide having a sequence as set forth in SEQ ID NO:1 and 3; (b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.

11. A method of conferring nematode resistance to a plant, said method comprising the steps of: (a) selecting a target gene from the group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene; (b) preparing a nucleic acid sequence comprising a region that is substantially identical to a portion of the selected target gene, wherein the nucleic acid is able to form a double-stranded transcript once expressed in the plant; (c) transforming a recipient plant with said nucleic acid; (d) producing one or more transgenic offspring of said recipient plant; and (e) selecting the offspring for nematode resistance.

12. The method of claim 11, wherein the target gene is a polynucleotide selected from the group consisting of (a) a polynucleotide having a sequence as set forth in SEQ ID NO:1 and 3; (b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.

13. An expression cassette comprising a sequence substantially identical to a portion of a plant parasitic nematode target gene selected from a group consisting of a parasitic nematode innexin-like gene, a parasitic nematode gene encoding a polymerase delta small subunit (pol delta S), a parasitic nematode gene homologous to the C. elegans tcp-1 gene, a parasitic nematode gene homologous to a C. elegans pas-1 gene, a parasitic nematode snurportin-1 like gene, a parasitic nematode gene homologous to the C. elegans rpt-1 gene, a parasitic nematode gene encoding a 26S proteasome regulatory subunit 4 (prs-4), and a parasitic nematode gene homologous to a C. elegans rpn-5 gene.

14. The expression cassette of claim 13, wherein the target gene is a polynucleotide selected from the group consisting of (a) a polynucleotide having a sequence as set forth in SEQ ID NO:1 and 3;
(b) a polynucleotide having a sequence as set forth in SEQ ID NO:5, 7, 9, 72, 73, 74, 75, 76, 77 and 78; (c) a polynucleotide having a sequence as set forth in SEQ ID NO:11, 13, 15, 86, 87, 88, 89, 90 and 91; (d) a polynucleotide having a sequence as set forth in SEQ ID NO:19 and 21; (e) a polynucleotide having a sequence as set forth in SEQ ID NO:23 and 25; (f) a polynucleotide having a sequence as set forth in SEQ ID NO:104, 27, 29, 35, 37, 92, 93, 106 and 107; (g) a polynucleotide having a sequence as set forth in SEQ ID NO:39, 41, 43, 45, 47, 49, 51, 94, 95, 96, 97, 98, 99, 100, 101, 102 and 103; (h) a polynucleotide comprising a sequence as set forth in SEQ ID NO:57, 59, 61, 63, 79, 80, 81, 82, 83, 84 and 85.


SEQUENCE LISTING (107 sequences)

SEQ ID NO: 1 - DNA, 1844 bp, Heterodera glycines
ggacactgacatggactgaaggagtagaaaggtttaattacccaagtttgagcgccgctgtcactggcacagagccgctgacccgtacggcggccgctgcaacagcgggcgccaccaccgacatgttcttccacgcgacgctggcgcgcactttcatttccgcgctgcgcgttcgcggcgacgacgatgtggtggaccgtctaaactactactacacgccgatcatgttggccatcgcgtgccttgtcgtgtccgccaagcagttcggcggctcgcccatcgaatgttgggtgaacccgcactcgcgcgaaagcatggaggagtacatagaggcattctgctggatccagaacacttattgggtgccaatgtatgagcacattccggacagtcatgaagcgcgagaaggacagcaaattggctattaccaatgggtgccgttcattctgatcgcccaagcactgatgttctctttgccatgcattctgtggcgattgctcaattggcaaaatggcaccaacattcagcaactgatttcggctgcttgtgaggcgcgttcagtgatcgacgcggatgagagggaacgcgtggtgggcgcggtggcgcggacattcgtcgaaatgttggacctgcgcgaaattcaaaatcggccccacccttacgcttcatccctcgcccgtttcaacccaattcggctgatgaatggccatttggtctgttccctgtatttgttcaccaaagtgtgctattccgtcaacattatgctccaatttgctcttctcaatgccgcactgacctcaaaagaccattttctgtttgggtttcaagttctgtccgacctttacgagggaaaaccgtggacaaagtcgggccattttccacgagtgactctctgtgacttcgaagtgcgttatttggccaatttgaacagatacactgtacaatgcgctctgatcattaacattatcaacgaaaaggtgttcgctttcttttggctttggtactgtctgcttttatgtgccacaacctgttccgccctgttttggctcagcaacattctgttgcacatcgcccgggtggactacgtgctgaagtttatgcaaattgctgaacacagtgaacaacagcgaagcagcggcagaacgccaaaactatcgcaacaaaagtgggcgatggtggaggagggcgaaatgccacaattcaccaaaagaccatttcgggtgccgagtgcgcattcggtggacaaatttgtggacgaattcctcaaatcggacgggctgttcattctccgattggtggccacaaatgcgggcgaattggtcgtcgttgacattgtcaagtgcctttggagagagttttcttcccgtcagtttcatattcgtccgttggtctatgaaaacgaactgagcgaagaacggaggcgcgaggatgaagacagtcaccacagccttctgctgaatgtgtacagcagcagagggaacggaccgacccatcaacagcagcagaggaagcaaagccaacaacttcgatattccactaatggcaacagcctcggccttccactcccgacaatgtctcggccgccttctgtggtcgcattggacgattctgtcggcacaccgtcgcccgtctgatgaggaggagaaagaagcaaatggccgaagacattctcaacgaatggatgcgatgccatcagattaagaacaacattattattttaatgcgaccttttcacattttgtacataaatgtaattaaatacaatgtcagtcaaattgttaaagataataatttcaatttgaatataaagtgattggttgtcaaaaaaaaaaaaaaaaaaaaaa

SEQ ID NO: 2 - protein, 503 aa, Heterodera glycines
MFFHATLARTFISALRVRGDDDVVDRLNYYYTPIMLAIACLVVSAKQFGGSPIECWVNPHSRESMEEYIEAFCWIQNTYWVPMYEHIPDSHEAREGQQIGYYQWVPFILIAQALMFSLPCILWRLLNWQNGTNIQQLISAACEARSVIDADERERVVGAVARTFVEMLDLREIQNRPHPYASSLARFNPIRLMNGHLVCSLYLFTKVCYSVNIMLQFALLNAALTSKDHFLFGFQVLSDLYEGKPWTKSGHFPRVTLCDFEVRYLANLNRYTVQCALIINIINEKVFAFFWLWYCLLLCATTCSALFWLSNILLHIARVDYVLKFMQIAEHSEQQRSSGRTPKLSQQKWAMVEEGEMPQFTKRPFRVPSAHSVDKFVDEFLKSDGLFILRLVATNAGELVVVDIVKCLWREFSSRQFHIRPLVYENELSEERRREDEDSHHSLLLNVYSSRGNGPTHQQQQRKQSQQLRYSTNGNSLGLPLPTMSRPPSVVALDDSVGTPSPV

SEQ ID NO: 3 - DNA, 509 bp, Heterodera glycines
acattcgtcgaaatgttggacctgcgcgaaattcaaaatcggccccacccttacgcttcatccctcgcccgtttcaacccaattcggctgatgaatggccatttggtctgttccctgtatttgttcaccaaagtgtgctattccgtcaacattatgctccaatttgctcttctcaatgccgcactgacctcaaaagaccattttctgtttgggtttcaagttctgtccgacctttacgagggaaaaccgtggacaaagtcgggccattttccacgagtgactctctgtgacttcgaagtgcgttatttggccaatttgaacagatacactgtacaatgcgctctgatcattaacattatcaacgaaaaggtgttcgctttcttttggctttggtactgtctgcttttatgtgccacaacctgttccgccctgttttggctcagcaacattctgttgcacatcgcccgggtggactacgtgctgaagtttatgcaaattgctgaacacag

SEQ ID NO: 4 - protein, 169 aa, Heterodera glycines
TFVEMLDLREIQNRPHPYASSLARFNPIRLMNGHLVCSLYLFTKVCYSVNIMLQFALLNAALTSKDHFLFGFQVLSDLYEGKPWTKSGHFPRVTLCDFEVRYLANLNRYTVQCALIINIINEKVFAFFWLWYCLLLCATTCSALFWLSNILLHIARVDYVLKFMQIAEH

SEQ ID NO: 5 - DNA, 864 bp, Heterodera glycines
tgaaaacaatgactcgtgggtcaagcgccggatacgaccgtcacatcacaattttctcaccagaaggaaggatttaccaagttgaatacgcgttcaaagctgctaacactgcgacactctccgctgttggcattgctcttgatgaaactgccgtaattgccgtacaacgtcgcgttcccgacaaattggtggacccatcttcagttaaaagcatttataaattgtcctcaaccgtcagttgcggtgttatcggcattgtcccggatgcgatgttccaagtgcgccgtgcacaatcggaagcagccagatggaagtatgagaatggctatgaaatgccaatttcggaactcgcgcgtaaaatggccgagatcaaccagtactatacacaggttgctgaacttcgttcattgggcacactgatgcttatgatctcgtacgacgacgaaaaaggcgcttctgttttttccatcgatccagcaggacattacatttctgttcgtggttacggcattggagtcaagcagcagcagatcaacggttttctggagaaaaaactcaaatcaaaagaccgaaaattcggtgacacggaagtgattcaactggcgcttgaggcactgcaaaccgggctgggaattgacttaaaggctgacgaggttgaagtgattgttgctacaaaaacggacccgaaaggagtcaaagtgagcgacaaaagcattgaggagcatctgacggcaattgcggagagagactgaggacgaaagtgatttaaaaacaacattttgtggtgttatttttcgctcttctaattatggttttaacagaaaaaaatttgtttttcaaaaaaaaaaaaaaaaaaaaaaaaaa

SEQ ID NO: 6 - protein, 247 aa, Heterodera glycines
MTRGSSAGYDRHITIFSPEGRIYQVEYAFKAANTATLSAVGIALDETAVIAVQRRVPDKLVDPSSVKSIYKLSSTVSCGVIGIVPDAMFQVRRAQSEAARWKYENGYEMPISELARKMAEINQYYTQVAELRSLGTLMLMISYDDEKGASVFSIDPAGHYISVRGYGIGVKQQQINGFLEKKLKSKDRKFGDTEVIQLALEALQTGLGIDLKADEVEVIVATKTDPKGVKVSDKSIEEHLTAIAERD

SEQ ID NO: 7 - DNA, 530 bp, Heterodera glycines
caatgactcgtgggtcaagcgccggatacgaccgtcacatcacaattttctcaccagaaggaaggatttaccaagttgaatacgcgttcaaagctgctaacactgcgacactctccgctgttggcattgctctcgatgaaactgccgtaattgccgtacaacgtcgcgttcccgacaaattggtggacccatcttcagttaaaagcatttataaattgtcctcaaccgtcagttgcggtgttatcggcattgtcccggatgcgatgttccaagtgcgccgtgcacaatcggaagcagccagatggaagtatgagaatggctatgaaatgccaatttcggaactcgcgcgtaaaatggccgagatcaaccagtactatacacaggttgctgaacttcgttcattgggcacactgatgcttatgatctcgtacgacgacgaaaaaggcgcttctgttttttccatcgatccagcaggacattacatttctgttcgtggttacggcattggagtcaagcagcagcagatcaac

SEQ ID NO: 8 - protein, 176 aa, Heterodera glycines
MTRGSSAGYDRHITIFSPEGRIYQVEYAFKAANTATLSAVGIALDETAVIAVQRRVPDKLVDPSSVKSIYKLSSTVSCGVIGIVPDAMFQVRRAQSEAARWKYENGYEMPISELARKMAEINQYYTQVAELRSLGTLMLMISYDDEKGASVFSIDPAGHYISVRGYGIGVKQQQIN

SEQ ID NO: 9 - DNA, 615 bp, Globodera rostochiensis
atttgttaaaaaatgacacgcgggtcaagcgccggctacgatcgccacatcactattttctcaccggaagggcggatttatcaagttgaatatgcgttcaaagctgtaaacgcagcgactctatctgctgttggcgttgctcttgatgatactgccgtaattgccgtccagcgacgcattccagataagttggtcgacccctcctctgtcaagagcatctataagttgtcgtcaacggtcagttgcggtgttatcggcattgttccggacgcggtgttccaagtgcgtcgtgctcaatcagaggccgccaagtggaaatatgagaatggttatgaaatgccaatctctgagctggcacgcaaaatggccgagatcaaccagtactacacgcaggtggcggagcttcgttctctgggcaccctaatgctcatggtctcctttgatgatgagaaaggtgccagtgtctactctgtggatcctgcagggcattttatctctgttcgcggctatggcatcggcgtcaagcaacagctggtcaacggctttctggagaagaaactgaaggcaaaagaccgcaaattcggagaggcggaggtcattcaacttgcgcttgaa

SEQ ID NO: 10 - protein, 201 aa, Globodera rostochiensis
MTRGSSAGYDRHITIFSPEGRIYQVEYAFKAVNAATLSAVGVALDDTAVIAVQRRIPDKLVDPSSVKSIYKLSSTVSCGVIGIVPDAVFQVRRAQSEAAKWKYENGYEMPISELARKMAEINQYYTQVAELRSLGTLMLMVSFDDEKGASVYSVDPAGHFISVRGYGIGVKQQLVNGFLEKKLKAKDRKFGEAEVIQLALE

SEQ ID NO: 11 - DNA, 1791 bp, Heterodera glycines
aggctcttctgtagtccttaagcaacaaaaaaagtcgtagcttacttattattaatttgcattcaatgaatcctgttagaatattaaagcaaaatgcccaggaggaacgtggagaaactgcgagactttcttcatttgttggggcatgtgccattggtgacttggtcaaaacgactttagggcctaaagggatggacaaaattctcgtcagcggtaggggcgaacaccaaaatgttcaagtgacaaacgatggtgcgacaattctgaaatcgatcggtgttgataaccctgcagcaaaagttcttgtcgatatttctctgacccaggacaaggaagtgggcgatgggacaacgtcggtgactgtctttgcggccgaattgctcagagaggcggaagttatgattggacagcgaattcatccgcaagttattgtttccggctatcgaaaggctgttcgagttgcgaaggacgcacttgaaaatgctgcccaagcatcaggagagcatttgcgcgaagatttgctgaaaattgcgaagacttcactgggttccaagattctttcccagcattccaaccattttgccaaattggcggtcgatgccgtcctccgactcggccccaacggcgctttggactccatccaagtgattaaaaaactgggtggatcgatggaagattcgtaccttgacgagggatttttgctggagaagaaggccggcatgtaccaaccacagcgaattgaagacgccaaaattcttattgcaaattctccgatggaccaggacaaaataaaagttttcggaagcagaattcgagtggattcagtggcaaaaattgccgaattggaacaagcggaaaaagacaaaatgaagcagaaagtggagaatatttgcaatcacggcatcaatgtgttcatcaatcgtcagctcatttacaattaccccgaacaattgttcgctgatcggaaagtgatggccatcgaacacgccgactttgagggcatcgaacgattggcacttgtattaggtggcgaaattgcttcaacatttgacagtccatcggaggtgaaattgggtagctgcgaactcattgaagaagtcaccgttggcgaagacactttgctccgcttttccggtgtcccgcttggcaatgcatgttccgttgtgcttcgtgggtccacccagcaaatcattgatgaagcagaacgttcgttgcacgacgcgctttgtgtgctgagtacgcatgtgaaagaccaacgcgtggtgcccggggcgggcgcatcggaaatgctcatggcaatggcagtgatgggcgaaagtcaaaaggtggccgggaaggagtccatcgcaatggaagcattcgcacgggcactcgccaaattgcccacaatcatttgcgacaacgccggactggacagtgccgaaatcatttcgcatgtgcgagccgaacacagcaaagggaatcgccaatttggcattgatgttgaaaatggtcgtatggcggatgtttacgagttgggcgtgttggagtcgtacaacgttaaattgggcgtgctttgcagtggcgccgaagcggccgagcagcttctccgtgtcgattgcatcatcaaatgcgcgccgcgccctcgcacgaaggaccgtcgtccgtgctgagaaaacgcagctagcgaatgcgaattattgaagacgcgcacaggaaaaggaatacttcattgcttgctttaccgtactttgttattgttttttgacatgttaaaaccttaaaaaccgaaaaaaaaaaaaaaaaaaaaaa

SEQ ID NO: 12 - protein, 528 aa, Heterodera glycines
MNPVRILKQNAQEERGETARLSSFVGACAIGDLVKTTLGPKGMDKILVSGRGEHQNVQVTNDGATILKSIGVDNPAAKVLVDISLTQDKEVGDGTTSVTVFAAELLREAEVMIGQRIHPQVIVSGYRKAVRVAKDALENAAQASGEHLREDLLKIAKTSLGSKILSQHSNHFAKLAVDAVLRLGPNGALDSIQVIKKLGGSMEDSYLDEGFLLEKKAGMYQPQRIEDAKILIANSPMDQDKIKVFGSRIRVDSVAKIAELEQAEKDKMKQKVENICNHGINVFINRQLIYNYPEQLFADRKVMAIEHADFEGIERLALVLGGEIASTFDSPSEVKLGSCELIEEVTVGEDTLLRFSGVPLGNACSVVLRGSTQQIIDEAERSLHDALCVLSTHVKDQRVVPGAGASEMLMAMAVMGESQKVAGKESIAMEAFARALAKLPTIICDNAGLDSAEIISHVRAEHSKGNRQFGIDVENGRMADVYELGVLESYNVKLGVLCSGAEAAEQLLRVDCIIKCAPRPRTKDRRPC

SEQ ID NO: 13 - DNA, 500 bp, Heterodera glycines
ttcttattgcaaattctccgatggaccaggacaaaataaaagttttcggaagcagaattcgagtggattcagtggcaaaaattgccgaattggaacaagcggaaaaagacaaaatgaagcagaaagtggagaatatttgcaatcacggcatcaatgtgttcatcaatcgtcagctcatttacaattaccccgaacaattgttcgctgatcggaaagtgatggccatcgaacacgccgactttgagggcatcgaacgattggcacttgtattaggtggcgaaattgcttcaacatttgacagtccatcggaggtgaaattgggtagctgcgaactcattgaagaagtcaccgttggcgaagacactttgctccgcttttccggtgtcccgcttggcaatgcatgttccgttgtgcttcgtgggtccacccagcaaatcattgatgaagcagaacgttcgttgcacgacgcgctttgtgtgctgagtacgcatgtgaaagac

SEQ ID NO: 14 - protein, 166 aa, Heterodera glycines
LIANSPMDQDKIKVFGSRIRVDSVAKIAELEQAEKDKMKQKVENICNHGINVFINRQLIYNYPEQLFADRKVMAIEHADFEGIERLALVLGGEIASTFDSPSEVKLGSCELIEEVTVGEDTLLRFSGVPLGNACSVVLRGSTQQIIDEAERSLHDALCVLSTHVKD

SEQ ID NO: 15 - DNA, 585 bp, Heterodera schachtii
atcgagcgattggcactcgttttaggcatgtttcgtcgcaattcctattcctaatttgaaagggactgtttcctaactcaactttccataaggtggcgaaattgcttcaacatttgacagtccatcggaggtgaaattgggtagctgcaaactcattgaagaagtcaccgttggcgaagacactttgctccgcttttccggtgtcccgcttggcaatgcgtgttccgttgtgcttcgtgggtccacccagcaaatcattgatgaagcggaacgttcgttgcacgacgcgctttgtgtgctgagtacgcatgtgaaagaccaacgcgtggtgcccggggcgggcgcatcggaaatgctcatggcaatggcagtgatgggcgaaagtcaaaaggtggccgggaaggagtccatcgcaatggaagcattcgcacgggcactcgccaaattgcccacaatcatttgcgacaacgccggactggacagtgccgaaatcatttcgcatgtgcgagccgaacacagcaaagggaatcaccaatttggcattgatgttgaaaatggtcgtatggcggatgtttacgagttggg

SEQ ID NO: 16 - protein, 164 aa, Heterodera schachtii
GGEIASTFDSPSEVKLGSCKLIEEVTVGEDTLLRFSGVPLGNACSVVLRGSTQQIIDEAERSLHDALCVLSTHVKDQRVVPGAGASEMLMAMAVMGESQKVAGKESIAMEAFARALAKLPTIICDNAGLDSAEIISHVRAEHSKGNHQFGIDVENGRMADVYEL

SEQ ID NO: 17 - DNA, 1590 bp, Caenorhabditis elegans
atgcttccagtccaaattttaaaggacaatgctcaagaggaacgaggagagagcgctcgtctcagctcattcgtgggtgcaatcgccatcggagatcttgtcaagtctactcttggaccaaagggaatggacaaaattctcatcagtggaaacccagaaagcgcaggaggaatcaaagttaccaacgatggagcaacgatcctgaagtcaatcggtgttgacaatccagctgctaaggttcttgttgatatgtcaatgacacaggatcacgaagttggagatggtactacttcagtaactgtcctggccgcagagctcctcaaagaagccgagaaacttgtcaatcaacgtattcatccacagacgatcatttctggatatagacgtgctctcgggattgctcaagaatccttgaaaaagtccagcattgaatccggagataatattcgtgatgatcttttgaagattgcccgtactactcttggatctaagattctcagtcaacacaaagagcactttgctcaactcgccgttgatgcagttttgagactcaagggatctggaaatttggatgccattcaaattatcaagaagctcggaggatccatgaatgaatcttatctcgacgagggtttccttctcgaaaaactccctggaatgttccaaccaagaagagttgagaaagcaaagattctcattgccaacacaccaatggacactgacaaagtgaaagttttcggatcaagagtgagagtagatggtgttgccaaggttgctgaactcgaagctgctgagaaattgaagatgaaagaaaaagttgacaaaattctggcccataactgtaatgtcttcatcaaccgtcagttgatctacaactacccagagcaactttttgccgatgctaaggttatggctattgaacacgccgattttgaaggaatcgagaggcttgctcttgttctcggaggagaaattgtttccacatttgattcaccacaaactgctcaattcggatcttgtgatctgattgaagagattatgattggagaagatagacttcttcggttctcgggagtcaaattgggtgaagcttgctcagtcgttcttcgtggagcaactcagcagattcttgatgagtccgaaagatccctccatgatgccctttgtgttctcgttactcacgtgaaagaatcaaagactgttgccggagctggagcgagcgaaattttgatgagttcagccatcgccgtcgaagctcaaaaggttgcgggtaaggaatcccttgccgttgaagccttcggaagagcccttgctcaattgccaaccatcatttgcgacaatgctggacttgactcggctgaacttgtcacaagactccgtgcagagcacgccaatggacgtcacaatatgggaatcgacatcgagaaaggagaggttgctgatgttacgaaattgggcgttattgagtcttacaatgtgaagttgtgcatggtttcttccgccgctgaagccacggaacaaattcttcgcgtcgacgatatcatcaaggcagctccacgtgcccgtgctcaagataaccgaccatgctaa

SEQ ID NO: 18 - protein, 529 aa, Caenorhabditis elegans
MLPVQILKDNAQEERGESARLSSFVGAIAIGDLVKSTLGPKGMDKILISGNPESAGGIKVTNDGATILKSIGVDNPAAKVLVDMSMTQDHEVGDGTTSVTVLAAELLKEAEKLVNQRIHPQTIISGYRRALGIAQESLKKSSIESGDNIRDDLLKIARTTLGSKILSQHKEHFAQLAVDAVLRLKGSGNLDAIQIIKKLGGSMNESYLDEGFLLEKLPGMFQPRRVEKAKILIANTPMDTDKVKVFGSRVRVDGVAKVAELEAAEKLKMKEKVDKILAHNCNVFINRQLIYNYPEQLFADAKVMAIEHADFEGIERLALVLGGEIVSTFDSPQTAQFGSCDLIEEIMIGEDRLLRFSGVKLGEACSVVLRGATQQILDESERSLHDALCVLVTHVKESKTVAGAGASEILMSSAIAVEAQKVAGKESLAVEAFGRALAQLPTIICDNAGLDSAELVTRLRAEHANGRHNMGIDIEKGEVADVTKLGVIESYNVKLCMVSSAAEATEQILRVDDIIKAAPRARAQDNRPC

SEQ ID NO: 19 - DNA, 1508 bp, Heterodera glycines
ggacactgacatggactgaaggagtagaaaggtttaattacccaagtttgagcattttcatcaaacaaaaacctattattctctaaaaccaattaatggacgtggacgatttgctagattccttttccaaatcaacggtcacagcggacaacgaattcggtgtcaccagtgccaccgccgttggtggtgggggtgctgaggatggcattggcgcccagcagcagcagcagtgggaggcggcacagcacccgcattttggccgcaactacaaaaatgagggccgagtggccgcgctgcagcgccaacgccgtcaggaacacttggagagacagcatttggctcgcgaggattggctgaggcgccgccgtgaaattgaagatgatgagtcgtcgtcatttttgcgtcgtgtgcggcagaaaaggcagaacccgtacaaggacatgttgatgttcagcgattggcttgttgacattccaggcactttgtccaccgagtggacaatgcttccgtcgccggtcggacgtcgcactttggttgtggccaacagaggcgaaacgcgagtgtacttcaaaaatgggcatttggccaccaatttccattcacttttgccgggaggaaatgccaaaacgaaaggttctctgaccattttggacgccatttttgacgcgaagaagcggaagttgtacctgcttgatttgctctggtggaacaagctgatgtacacggacatggaattcaccgcgcgtcgcttctttctccagtcgcgcattgacgaaatgaacgaggacattgagcggaaaaacagcagggccagcatcagcaaaaatcaggaacgaaatggttgcaaaattgcggagcaggacaaaatgtcgtcttccgaaatctcgccgtacgaaatgtcaccgccgagcgacacgtcgcctgaacaaaacgccgtcgagccaaaatctcgccgtgacattaaatttgtgcctgtgccctcttgcgcttgttctccggacgaaattggccaatttatgcgcaccctttttacattccgtatcgacgggctgctgttctactacaactcggccttttacatccctgaacaggtcgccgaattttgttttcaattgtttctttttgaatttttcctcgtttttcgagacgcccctcgttggttggcttaagccgtggatgcttccagaggtccttggagtgccggtgcccgagctgtacaaggaggaaatggcatgcggaagttcccaggagttcattgaccagttcaacaaggagcacgggcacgtttcgtcggcggaaaaatatcggcagaaagcgcagtcgccggacatgacaatggacgaggcgaacgcaaatgccgacgaatggacggagggcaaagagatgggcacggcatggaaggaggagggagaagagcagagcaaaaatggaggataaagagatgaaaacggcacaagagaagcggacggaccgagacttcggcactttttgacccaaaaaaaaaaaaa

SEQ ID NO: 20 - protein, 357 aa, Heterodera glycines
MDVDDLLDSFSKSTVTADNEFGVTSATAVGGGGAEDGIGAQQQQQWEAAQHPHFGRNYKNEGRVAALQRQRRQEHLERQHLAREDWLRRRREIEDDESSSFLRRVRQKRQNPYKDMLMFSDWLVDIPGTLSTEWTMLPSPVGRRTLVVANRGETRVYFKNGHLATNFHSLLPGGNAKTKGSLTILDAIFDAKKRKLYLLDLLWWNKLMYTDMEFTARRFFLQSRIDEMNEDIERKNSRASISKNQERNGCKIAEQDKMSSSEISPYEMSPPSDTSPEQNAVEPKSRRDIKFVPVPSCACSPDEIGQFMRTLFTFRIDGLLFYYNSAFYIPEQVAEFCFQLFLFEFFLVFRDAPRWLA

SEQ ID NO: 21 - DNA, 160 bp, Heterodera glycines
gaattcaccgcgcgtcgcttctttctccagtcgcgcattgacgaaatgaacgaggacattgagcggaaaaacagcagggccagcatcagcaaaaatcaggaacgaaatggttgcaaaattgcggagcaggacaaaatgtcgtcttccgaaatctcgccgt

SEQ ID NO: 22 - protein, 53 aa, Heterodera glycines
EFTARRFFLQSRIDEMNEDIERKNSRASISKNQERNGCKIAEQDKMSSSEISP

SEQ ID NO: 23 - DNA, 1686 bp, Heterodera glycines
ttgtcatccatttatttgtgcttatattgtgcaatcaatatttgccctttgccctccgtccctcttcgcttatttttttattttgtgtcttcacaacttgaataaaataattgtttatacacttcgccgctgacttatggtgaagcaacaggaacgcatatcttttcccttctcagttgactcaaagcgttttttacttttgcccaccgattttgtggatgaaaaaagcaaatttttcaaccgtcagtttctcaccatttatcgggcacggattaattgtcttaaagacttaattaaaaaaaatgcgcgaaatattcttgcttctacctccaataattctgttcaaatcgatgatttatcaaatttttctgccggcaatgatattttgctgatcggtgttgttttcaagaaaatgaaattccgtcaaagcattctttacgagttttcggatgattcgaatgttcccattaaaatggggagaaaagcgggcgacaacctttgcgatgacgaggacatactccaattggaggacgaccagcagactgttaaattgcttggaaacattgacaaacattgttttgtcactggcgatgtcattggagtgatcggctgtcaggaggatgtatcagacaattttgaagtgcgaatgattatttacccggaaatgagccctcaattggaatggcctttggttgagcatgattgttatattgtttttatgtccggcatttcattagttggcaattttgacaacgatgtccaaacgttttcggcactgatgcagtttcagcggtggataaacggggaggtggaggtgtcaaaggacggcactgatttgagtgatgagggcgaggacgagtcggacactttgcgcaacatcgccagacttgtcattgctggtgattttgcgcgttttgcacagaatgacattgaaactcagcgagtttcgatgattggcgccgaacttgactcggacatggactctttttcgcaatttgacaaatttttggccactcttttgcaaaatttgagagttgacttaatgccaggagccagtgatcccgtccagtgcatgatcccccaacagccaattccccccgcggtgttcactttggcggcgccgttccaaccaatgcttaacacagtgaccaatccctacagtttcgagctcaacggagtccgttttttggggacatcaggtcaaaacatcaatgatttgcggcggttgacacgcggaaaggacacattagcattgatggaacgcacgttggaaatgggctatattttcccgacagtccccgacactcttcccggatttcctttttctggacgcgacccgcttgttttggaccaaattccgcacatttatttcgtcgggaatcagccaacctttgaaaaacgaatcgttgaatttggcggaaaaccaacgaaaagatgttgccttttggctgttcctaagttttgcaagacaaaatcagttattctgctgaatttacggactcttgaatccaatgaatactgttttggtgcaaatttcaatgaatcaggtcaatagaagttcgagaagggtcatttgatctcaattttatgcttgcatttatttaaaagcgattggccaattactccaaacgtcactttcattgataaacacataaaattgcaaaaaaaaaaaaaaaaaaaaaaaaa

SEQ ID NO: 24 - protein, 472 aa, Heterodera glycines
MVKQQERISFPFSVDSKRFLLLPTDFVDEKSKFFNRQFLTIYRARINCLKDLIKKNARNILASTSNNSVQIDDLSNFSAGNDILLIGVVFKKMKFRQSILYEFSDDSNVPIKMGRKAGDNLCDDEDILQLEDDQQTVKLLGNIDKHCFVTGDVIGVIGCQEDVSDNFEVRMIIYPEMSPQLEWPLVEHDCYIVFMSGISLVGNFDNDVQTFSALMQFQRWINGEVEVSKDGTDLSDEGEDESDTLRNIARLVIAGDFARFAQNDIETQRVSMIGAELDSDMDSFSQFDKFLATLLQNLRVDLMPGASDPVQCMIPQQPIPPAVFTLAAPFQPMLNTVTNPYSFELNGVRFLGTSGQNINDLRRLTRGKDTLALMERTLEMGYIFPTVPDTLPGFPFSGRDPLVLDQIPHIYFVGNQPTFEKRIVEFGGKPTKRCCLLAVPKFCKTKSVILLNLRTLESNEYCFGANFNESGQ

SEQ ID NO: 25 - DNA, 224 bp, Heterodera glycines
cgcgacccgcttgttttggaccaaattccgcacatttatttcgtcgggaatcagccaacctttgaaaaacgaatcgttgaatttggcggaaaaccaacgaaaagatgttgccttttggctgttcctaagttttgcaagacaaaatcagttattctgctgaatttacggactcttgaatccaatgaatactgttttggtgcaaatttcaatgaatcaggtcaata

SEQ ID NO: 26 - protein, 74 aa, Heterodera glycines
RDPLVLDQIPHIYFVGNQPTFEKRIVEFGGKPTKRCCLLAVPKFCKTKSVILLNLRTLESNEYCFGANFNESGQ

SEQ ID NO: 27 - DNA, 575 bp, Heterodera glycines
aaggacaggaccctgaaaaaaaacggaaataccatggaccgccggttccaacgcgaattggaaaacgcaagaagggctctcgtggtcccgacacagcaaacaaaatgcccaccgtgactccgatcactcgttgtaaactcaagctcctcaagtatgaccggattaaggactatcttttaatggaggaagaattcataaagaacatggagcgtttgaagcctcaggacgaacgtcaggaggaagagcgtgttaaagttgacgaccttcgtgggactccaatgtctgtcggatcattggaagaagtcattgacgatcaacacgcaattgtttccacgaatgtcggcagtgaacattacgtcaacattttgtcatttgtggacaaagaacagcttgagccgggctgtgccgttctgctgaatcacaaaacccactcaatcgtcggcgttcttgcggaggacgccgatccgatggtgtcggtgatgaaattggagaaagcaccgaccgagacttacgctgacgttggtggcctcgagcagcaaattcaggagatcaaagaggcggtggagttgccgttg

SEQ ID NO: 28 - protein, 191 aa, Heterodera glycines
GQDPEKKRKYHGPPVPTRIGKRKKGSRGPDTANKMPTVTPITRCKLKLLKYDRIKDYLLMEEEFIKNMERLKPQDERQEEERVKVDDLRGTPMSVGSLEEVIDDQHAIVSTNVGSEHYVNILSFVDKEQLEPGCAVLLNHKTHSIVGVLAEDADPMVSVMKLEKAPTETYADVGGLEQQIQEIKEAVELPL

SEQ ID NO: 29 - DNA, 498 bp, Heterodera glycines
taccatggaccgccggttccaacgcgaattggaaaacgcaagaagggctctcgtggtcccgacacagcaaacaaaatgcccaccgtgactccgatcactcgttgtaaactcaagctcctcaagtatgaccggattaaggactatcttttaatggaggaagaattcataaagaacatggagcgtttgaagcctcaggacgaacgtcaggaggaagagcgtgttaaagttgacgaccttcgtgggactccaatgtctgtcggatcattggaagaagtcattgacgatcaacacgcaattgtttccacgaatgtcggcagtgaacattacgtcaacattttgtcatttgtggacaaagaacagcttgagccgggctgtgccgttctgctgaatcacaaaacccactcaatcgtcggcgttcttgcggaggacgccgatccgatggtgtcggtgatgaaattggagaaagcaccgaccgagacttacgctgacgttggtggc

SEQ ID NO: 30 - protein, 166 aa, Heterodera glycines
YHGPPVPTRIGKRKKGSRGPDTANKMPTVTPITRCKLKLLKYDRIKDYLLMEEEFIKNMERLKPQDERQEEERVKVDDLRGTPMSVGSLEEVIDDQHAIVSTNVGSEHYVNILSFVDKEQLEPGCAVLLNHKTHSIVGVLAEDADPMVSVMKLEKAPTETYADVGG

SEQ ID NO: 31 - DNA, 1332 bp, Caenorhabditis briggsae
atgggacagcaacagtctggcttcggaggaagaggtaacgaccggggatccggtgataacgaaaagaaagaaaagaagaagtatgaggctccaattccatcaagaatcggaaaaaagaagaagggatctaaaggaccagatgccgcaagcaaattgccagcagttaccccacacgccagatgccgtctgaagttgttgaaaagtgagagaatcaaggattacttgctgatggagcaggaattcatccaaaaccaagagcgtctgaagccacaagaggagagacaggaggaagaacgtgctaaggttgatgagcttcgtggaactccaatggctgttggatcattggaagaaattattgatgatcaacatgctattgtttctactaacgtcggaagtgagcattatgtcaacatcatgtcattcgtagacaaggaacagttggaaccaggatgctctgtacttctcaaccacaaaaatcatgcagttatcggagtactttccgatgacactgacccaatggtatctgttatgaaactcgagaaagccccacaggaaacctacgcagatgttggaggactcgatcaacaaattcaagaaatcaaagaagctgttgaattgcctcttacccatcctgaatactatgaagaaatgggtattcggccgccaaagggagttatcctctacggttgccctggtactggaaagacccttctagcgaaagccgtagcgaatcaaacatcagcaactttcctccgtattgttggatcagagcttatccagaaatacctcggagacggaccaaaaatggttcgtgagcttttccgcgtcgccgaggaaaatgcaccatcaatcgtcttcatcgacgaaatcgacgctgttggaacgaagcgttacgattccaactctggaggagaacgggaaattcaacgtaccatgcttgagcttctaaatcagctagacggattcgactctcgtggagacgtgaaagtgctcatggctacaaaccgtatcgagtctctcgaccctgcattgatccgtccaggacgtattgatcgtaaaatcgagttcccattgccagatgagaaaaccaagcgccgcatcttccagattcacacatcacgcatgacccttggagacgatgtgaacctcgaagaattcatcactgctaaggatgagttgagtggtgctgacatcaaggcaatgtgtacagaagctggtctcctggctctccgtgaacgtcgaatgcgtgtgaccatggaggatttccagaagtccaaagaaaatgtcctttaccgcaagaaggaaggagcccccgaagagctctacttgtaa

SEQ ID NO: 32 - protein, 443 aa, Caenorhabditis briggsae
MGQQQSGFGGRGNDRGSGDNEKKEKKKYEAPIPSRIGKKKKGSKGPDAASKLPAVTPHARCRLKLLKSERIKDYLLMEQEFIQNQERLKPQEERQEEERAKVDELRGTPMAVGSLEEIIDDQHAIVSTNVGSEHYVNIMSFVDKEQLEPGCSVLLNHKNHAVIGVLSDDTDPMVSVMKLEKAPQETYADVGGLDQQIQEIKEAVELPLTHPEYYEEMGIRPPKGVILYGCPGTGKTLLAKAVANQTSATFLRIVGSELIQKYLGDGPKMVRELFRVAEENAPSIVFIDEIDAVGTKRYDSNSGGEREIQRTMLELLNQLDGFDSRGDVKVLMATNRIESLDPALIRPGRIDRKIEFPLPDEKTKRRIFQIHTSRMTLGDDVNLEEFITAKDELSGADIKAMCTEAGLLALRERRMRVTMEDFQKSKENVLYRKKEGAPEELYL

SEQ ID NO: 33 - DNA, 1332 bp, Caenorhabditis elegans
atggggcaacaacagtcaggtttcggtggaagaggaaatgaccgcggtgccggagatggagagaagaaggaaaagaagaaatatgaggcaccgattccatctaggattggaaagaagaagaaggggtctaagggacctgacgccgctagcaaacttccagctgtcacaccacatgccagatgccgtctgaagttgctgaagagcgaaagaatcaaggattatttgttgatggagcaagaattcattcaaaaccaggagcgtttgaagccacaagaggagagacaagaagaagagcgcgccaaggtggacgagctccgtggaacaccaatggcagtcggatcacttgaggaaattatcgatgatcagcacgccattgtttcaactaacgtcggaagtgagcattacgtcaacatcatgtcattcgtcgacaaggaacaactcgagccgggatgttcagtccttctcaaccacaagaatcacgccgtcatcggagttctctccgatgatacagatcctatggtgtctgtcatgaagctcgaaaaagcaccacaagaaacgtatgccgatgttggtggtcttgatcagcaaattcaagaaattaaagaagcagttgagcttccactgactcatcctgaatactacgaagagatgggaatccggcctccaaagggagttattctatacggttgtcctggtactggaaaaacgctacttgccaaggctgttgctaatcagacgtcagctactttccttcgtattgttggttcggaacttattcagaaatacctcggagatggtccaaagatggtccgtgagctcttccgcgttgctgaggagaatgcaccatcaattgtgttcattgatgaaattgatgctgttggaacaaaacgttacgattcgaactctggtggagaacgtgaaattcagcggactatgcttgaacttctcaatcaacttgatggattcgattcccgtggagatgttaaagttttgatggccactaaccgcatcgaatctctcgaccctgctctcattcgtccaggtcgtattgatcgtaagattgaattccccctgccagatgaaaaaacgaagcgccgtatcttccagatccatacatctcgaatgacactcggcaaagaagttaacctcgaagagttcatcacagcaaaggacgagttgagtggagctgatatcaaggctatgtgcacagaagctggtctcttagctcttcgcgagcgtcgtatgcgtgtcacgatggaagatttccagaagtccaaagaaaatgtgctctaccgtaagaaggaaggagctccagaagaactctatttgtaa

SEQ ID NO: 34 - protein, 443 aa, Caenorhabditis elegans
MGQQQSGFGGRGNDRGAGDGEKKEKKKYEAPIPSRIGKKKKGSKGPDAASKLPAVTPHARCRLKLLKSERIKDYLLMEQEFIQNQERLKPQEERQEEERAKVDELRGTPMAVGSLEEIIDDQHAIVSTNVGSEHYVNIMSFVDKEQLEPGCSVLLNHKNHAVIGVLSDDTDPMVSVMKLEKAPQETYADVGGLDQQIQEIKEAVELPLTHPEYYEEMGIRPPKGVILYGCPGTGKTLLAKAVANQTSATFLRIVGSELIQKYLGDGPKMVRELFRVAEENAPSIVFIDEIDAVGTKRYDSNSGGEREIQRTMLELLNQLDGFDSRGDVKVLMATNRIESLDPALIRPGRIDRKIEFPLPDEKTKRRIFQIHTSRMTLGKEVNLEEFITAKDELSGADIKAMCTEAGLLALRERRMRVTMEDFQKSKENVLYRKKEGAPEELYL

SEQ ID NO: 35 - DNA, 1263 bp, Meloidogyne hapla
aaaaagaaacgccgttttggacctccaatctcgatacgttttggaaagcgaaagaagggaagtaaaggacctgaagctagcaataaaatgccaaatgtggcaccattaacacgttgcaagttacgtcttcttaaatatgaacgtattaaagattatttgttgatggaagaagaatttattcgaaatcaggaaagacttaaacctcaagaggaaagacaagaagaagaacgtacaaaggtcgatgaaatgagagggtcaccaatggctgtgggtacattagaggaggttattgacgatcaacatgcaattttttcaacgaacgttggcagtgaacattatgttaacattctttcatttgtagacaaggaacaactggagccaggatgtgcagttcttctaaatcacaagacacatgcagttgttggtgttttggctgatgacacagacccaatggtttcagtaatgaaattagaaaaggctccaactgaaacatacgctgatgttggtggtttggaacaacaaattcaagaaattaaagaggcagtagaattgccacttacacatccagaatattacgaagaaattggaatcaaacctccaaaaggagttattctttacggcccacctggcacaggcaaaacattgctagctaaggctgtagctaatcaaacatctgctacctttttgcgtgtagttggttctgaacttattcaaaaatatttgggggatggacctaaaatggttagagaactctttcgggttgctgaagaacatgcaccgtcaattgttttcatcgacgaaattgatgctatcggcacaaaacgttatgaatccaattctggtggagaacgtgaaattcaacgtactatgcttgaattgctcaatcaattggatggttttgattctcgtggggatgttaaggttttaatggccacaaatcgaattgattcattagacccagcacttattcgccctggacgtattgaccgaaaaattgaatttcctcttcccgatgagaaaactaaaagacgaattttcggtattcacacagctcgtatgcaattggaaaatgttaatttggaggaatttattgaggcaaaggatgatctttcgggggctgatgttaaagcagtttgcaccgaggcagggcttttggcacttcgagatcgtcgaatgcgtgttactatggaggacatgaagaaagctaaagagaatgtactctacagaaagaaagatggtgccccagagtcgatgtacctttga

SEQ ID NO: 36 - protein, 420 aa, Meloidogyne hapla
KKKRRFGPPISIRFGKRKKGSKGPEASNKMPNVAPLTRCKLRLLKYERIKDYLLMEEEFIRNQERLKPQEERQEEERTKVDEMRGSPMAVGTLEEVIDDQHAIFSTNVGSEHYVNILSFVDKEQLEPGCAVLLNHKTHAVVGVLADDTDPMVSVMKLEKAPTETYADVGGLEQQIQEIKEAVELPLTHPEYYEEIGIKPPKGVILYGPPGTGKTLLAKAVANQTSATFLRVVGSELIQKYLGDGPKMVRELFRVAEEHAPSIVFIDEIDAIGTKRYESNSGGEREIQRTMLELLNQLDGFDSRGDVKVLMATNRIDSLDPALIRPGRIDRKIEFPLPDEKTKRRIFGIHTARMQLENVNLEEFIEAKDDLSGADVKAVCTEAGLLALRDRRMRVTMEDMKKAKENVLYRKKDGAPESMYL

SEQ ID NO: 37 - DNA, 658 bp, Meloidogyne incognita
atgggaaatcaacaattccaacaacaaaacccaggttcaggagataagcaaggagaaggagacaagaagcgtcgttttgggcctccaatccctacacgctttgggaagcgaaagaagggaagtaaaggacctgaagctagcaataaaatgcctaatgtgacaccagtaactcgttgcaaattgcgtcttcttaaatatgaacggattaaagattatttgttaatggaagaagaatttattcgaaatcaggaaagacataaacctcaagaagaaaggcaggaagaagaacggacaaaagtagatgaaatgagagggtcaccaatggctgtggggacattggaggagattattgacgatcaacacgcagtcgtttcgacgaacgttggaagcgaacattatgttaacattctttcatttgtggacaaggaacaattggagccaagatgtgcagttcttctaaatcacaaaacacacgcagttgttggtgttttggctgatgacaccgatccaatggtctctgtaatgaaattggaaaaggctccaaccgaaacatacgctgatgttggcgggttggaacaacaaattcaagaaattaaggaggcagtggaattgccacttacacatccagagtactacgaagaaattggaatcaagcctc

SEQ ID NO: 38 - protein, 219 aa, Meloidogyne incognita
MGNQQFQQQNPGSGDKQGEGDKKRRFGPPIPTRFGKRKKGSKGPEASNKMPNVTPVTRCKLRLLKYERIKDYLLMEEEFIRNQERHKPQEERQEEERTKVDEMRGSPMAVGTLEEIIDDQHAVVSTNVGSEHYVNILSFVDKEQLEPRCAVLLNHKTHAVVGVLADDTDPMVSVMKLEKAPTETYADVGGLEQQIQEIKEAVELPLTHPEYYEEIGIKP

SEQ ID NO: 39 - DNA, 1444 bp, Heterodera glycines
ctttgctttgttgaatttcttccactcaaaaatgtccagcgatattgtcgagaaaaaggagacaaaccccaatgagacggatgacaaaaccaaagaaataaaatcgcttgacgaggatgaaattgccgcacttagtaattacaacatgggaccgtacgcggatcagttgaagcaggcggagaaggacattgatgaaattcagaagcgcataaacactctttgcggagtgaaagagagcgacacggggctggcgccgcccattctttgggacattgcggccgacaaaatggccatgtcccatgagcagccgctgcaggtggctcgctgcacaaaaatcatcaaagaagagggcaaagaaacgcgttacatgatcaatgtgaagcagttcgccaagttcgtcgtggacctgcacgaaaatgtggcgcccactgacattgaggagggaatgcgagtgggtgtggaccgcaacaaataccagattcatttgcctttgccggcaaagattgacgcgtccgttacgatgatgcaagtggaggacaagccggacgttacctacgcggacattggcgggtgcgaagaacagatcaaaaagttgcgtgaagtggtcgagtttccgttgcttcagcctgagcgtttcacgagtttgggcattgagcctccgaagggcgttttgttttttggtccgccgggcaccggcaaaactttgtgtgcccgcgcggtcgccaatcggacggacgcgtgtttcatccgcgtcatcggttccgaattagtcaaaaaatacgttggcgaaggcgcgcgcatggtgcgcgagctgttttcgctggctaaaacgaaaaaggcgtgcattctcttcttcgacgaagtcgacgccatcggcggagcgcgatttgacgacggaaaagggggcgacaacgaagtgcaacggacgatgctcgagttggtcaaccaactggacggattcgactcacgcggggccatcaaggttttgatggccaccaacagaccggacacactcgacccggcgctcattcgtcccggtcgcattgaccgacgcattgaattttccttgcctgacctcaaggcacgaggaaacattctccaaattcacaccaaacggatgagcgtcgaccggaacattcggtacgaattgattgctcgactctgtccaaacacgacgggtgccgacttgcgcagcgtttgcactgaggcgggaatgttcgctttgcgtgcacgtcgaaaggtcataacggagcaagactttctcaaggctgttcagaaagtggtgaaaagttacgccaagttcagttcaacgccggcgtatatgacgcacaactgacaacacagttcttacaaaacggacttttttatatttgtgcacttttgtttcattacaatataaatgaggaaaccaaaaaaaaaaaaaaaaaaaaaaaaaaaaaaaaaa

SEQ ID NO: 40 - protein, 434 aa, Heterodera glycines
MSSDIVEKKETNPNETDDKTKEIKSLDEDEIAALSNYNMGPYADQLKQAEKDIDEIQKRINTLCGVKESDTGLAPPILWDIAADKMAMSHEQPLQVARCTKIIKEEGKETRYMINVKQFAKFVVDLHENVAPTDIEEGMRVGVDRNKYQIHLPLPAKIDASVTMMQVEDKPDVTYADIGGCEEQIKKLREVVEFPLLQPERFTSLGIEPPKGVLFFGPPGTGKTLCARAVANRTDACFIRVIGSELVKKYVGEGARMVRELFSLAKTKKACILFFDEVDAIGGARFDDGKGGDNEVQRTMLELVNQLDGFDSRGAIKVLMATNRPDTLDPALIRPGRIDRRIEFSLPDLKARGNILQIHTKRMSVDRNIRYELIARLCPNTTGADLRSVCTEAGMFALRARRKVITEQDFLKAVQKVVKSYAKFSSTPAYMTHN

SEQ ID NO: 41 - DNA, 368 bp, Heterodera glycines
ctttgctttgttgaatttcttccactcaaaaatgtccagcgatattgtcgagaaaaaggagacaaaccccaatgagacggatgacaaaaccaaagaaataaaatcgcttgacgaggatgaaattgccgcacttagtaattacaacatgggaccgtacgcggatcagttgaagcaggcggagaaggacattgatgaaattcagaagcgcataaacactctttgcggagtgaaagagagcgacacggggctggcgccgcccattctttgggacattgcggccgacaaaatggccatgtcccatgagcagccgctgcaggtggctcgctgcacaaaaatcatcaaagaagagagcaaagaaacgcgttaca

SEQ ID NO: 42 - protein, 122 aa, Heterodera glycines
FALLNFFHSKMSSDIVEKKETNPNETDDKTKEIKSLDEDEIAALSNYNMGPYADQLKQAEKDIDEIQKRINTLCGVKESDTGLAPPILWDIAADKMAMSHEQPLQVARCTKIIKEESKETRY

SEQ ID NO: 43 - DNA, 636 bp, Heterodera glycines
cacgaaaatgtggcgcccactgacattgaggagggaatgcgagtgggtgtggaccgcaacaaataccagattcatttgcctttgccggcaaagattgacgcgtccgttacgatgatgcaagtggaggacaagccggacgttacctacgcggacattggcgggtgcgaagaacagatcaaaaagttgcgtgaagtggtcgagtttccgttgcttcagcctgagcgtttcacgagtttgggcattgagcctccgaagggcgttttgttttttggtccgccgggcaccggcaaaactttgtgtgcccgcgcggtcgccaatcggacggacgcgtgtttcatccgcgtcatcggttccgaattagtcaaaaaatacgttggcgaaggcgcgcgcatggtgcgcgagctgttttcgctggctaaaacgaaaaaggcgtgcattctcttcttcgacgaagtcgacgccatcggcggagcgcgatttgacgacggaaaagggggcgacaacgaagtgcaacggacgatgctcgagttggtcaaccaactggacggattcgactcacgcggggccatcaaggttttgatggccaccaacagaccggacacactcgacccggcgctcattcgtcccggtcgcatt

SEQ ID NO: 44 - protein, 212 aa, Heterodera glycines
HENVAPTDIEEGMRVGVDRNKYQIHLPLPAKIDASVTMMQVEDKPDVTYADIGGCEEQIKKLREVVEFPLLQPERFTSLGIEPPKGVLFFGPPGTGKTLCARAVANRTDACFIRVIGSELVKKYVGEGARMVRELFSLAKTKKACILFFDEVDAIGGARFDDGKGGDNEVQRTMLELVNQLDGFDSRGAIKVLMATNRPDTLDPALIRPGRI

SEQ ID NO: 45 - DNA, 547 bp, Heterodera schachtii
caaagatacgcgttatgtgatcaatgtgaagcagttcgccaagttcgtcgtggacttgcacgaaaatgtggcgcccactgacattgaggagggaatgcgagtaggggtggaccgcaacaaataccagattcatttgcctttgccggcaaagattgacgcgtccgttacgatgatgcaagtggaggacaagccggacgttacctacgcggacattggcgggtgcgaagagcagatcaaaaagttgcgtgaagtggtcgagtttccgttgcttcagccggaacgtttcacgagtttgggcattgagcctccgaagggcgtcttgttttttggtccgccgggcaccggcaaaactttgtgtgcccgcgcggtcgccaatcggacggacgcttgtttcatccgcgtcatcggttccgaattggtcaaaaaatacgttggcgaaggcgcgcgcatggtgcgcgagctgttttcgctggcgaagacgaaaaaggcgtgcattctcttcttcgacgaagtcgacgccatcggcggagctcgttttgatgacgga

SEQ ID NO: 46 - protein, 182 aa, Heterodera schachtii
KDTRYVINVKQFAKFVVDLHENVAPTDIEEGMRVGVDRNKYQIHLPLPAKIDASVTMMQVEDKPDVTYADIGGCEEQIKKLREVVEFPLLQPERFTSLGIEPPKGVLFFGPPGTGKTLCARAVANRTDACFIRVIGSELVKKYVGEGARMVRELFSLAKTKKACILFFDEVDAIGGARFDDG

SEQ ID NO: 47 - DNA, 566 bp, Globodera rostochiensis
gaatcgaaccaccgaagggcgttctgttctttggtccgccgggcaccggcaaaacgctttgcgcccgcgcggtggccaatcggacggacgcgtgtttcattcgcgtcatcggttccgagctggtcaagaagtacgtcggcgaaggcgcccgcatggtgcgtgagctgttctcgcttgccaagacgaaaaaggcgtgcattctcttcttcgacgaggtggacgccatcggcggggcgcgctttgatgacggaaaaggcggcgacaacgaagtgcagcggacaatgctcgagctggtcaaccagctggacgggtttgattcgcgcggggccattaaggttttgatggccaccaacagaccggacacgctcgacccggccctcattcggcctggtcgtattgatcgacgcattgagttctgtttgcccgacctaaaggcccgtgggaacattcttcaaattcacaccaaacggatgagcgttgaccgaaacattcgatacgaattgattgcgcggctctgcccgaacacgacaggtgccgatttgcgaagcgtttgcactgaggcggga

SEQ ID NO: 48 - protein, 189 aa, Globodera rostochiensis
GIEPPKGVLFFGPPGTGKTLCARAVANRTDACFIRVIGSELVKKYVGEGARMVRELFSLAKTKKACILFFDEVDAIGGARFDDGKGGDNEVQRTMLELVNQLDGFDSRGAIKVLMATNRPDTLDPALIRPGRIDRRIEFCLPDLKARGNILQIHTKRMSVDRNIRYELIARLCPNTTGADLRSVCTEAG

SEQ ID NO: 49 - DNA, 583 bp, Globodera rostochiensis
ggcggggacaacgaagtgcagcggacaatgctcgagctggtcaaccagctggacgggtttgattcgcgcggggccattaaggttttgatggccaccaacagaccggacacgctcgacccggccttcattcggcctggtcgtattgatcgacgcattgagttctgtttgcccgacctaaaggcccgtgggaacatttttcaaattcacaccaaacggatgagcgttgaccgaaacattcgatacgaattgattgcggggttctgcccgaacacgacaggtgccgatttgcgaagcgtttgcactgaggcgggaatgttcgttttgcgcgcgcgtcgaaaggttatcacggagcaagacttcctcaaggccgtccaaaaagtggtgaaaagctatgggaagttcagctcaacaccggcctatatgacgcacaattaattggacattttgtcattttgatgaaatggacggttgataattttttgtttgtttacacacattcaattcgtcatttgttcattcgaaaagaagcgtggaaccgtcaaaaaaaaaaaaaaaaaccaaaaaagaaacatgtcggccgcct

SEQ ID NO: 50 - protein, 144 aa, Globodera rostochiensis
GGDNEVQRTMLELVNQLDGFDSRGAIKVLMATNRPDTLDPAFIRPGRIDRRIEFCLPDLKARGNIFQIHTKRMSVDRNIRYELIAGFCPNTTGADLRSVCTEAGMFVLRARRKVITEQDFLKAVQKVVKSYGKFSSTPAYMTHN

SEQ ID NO: 51 - DNA, 1297 bp, Meloidogyne hapla
aaggaagttaaagctttgggagaggaagaaatagccgctttaaaaagttacaatttggggccgtattctgagaaattgaggcaagtcgagactgacattcaagaagcgttaaagaatatcaacacactttgcggtgtcaaagaaagcgatactggccttgctcctcctgctctttgggatcttgttgctgataaaactgccattgctcaagagcaacctcttcaggttgcaagatgtacaaaaataattaaaactgagggtcaagatccacgttatatgattaatgtcaaacaatttgccaaatttgttgttgatttggcctctgcagttgctccaactgacattgaagaaggaatgcgtgttggtgttgatcgaaataaatatcagattcatattcctttaccagcaaaaattgatccttcagtgacaatgatgacagttgaagagaagccagatgtgacttatgctgacgttggtggttgtaaagaacaaatagaaaagcttcgagaagttgtcgaaatgcctctacttcatcctgaacgtttcgtgaatattggtattgaacctcctaagggtgtactcttttatggtcctcctgggacaggaaagacgctttgtgctcgtgctgttgcaaatcgaactgatgcttggtttattcgcgtaattggttctgaattggtacaaaaatatgttggtgaaggagcgagaatggttcgtgaattattcgagatggcaaaaacgaagaaggcttgtattattttctttgatgaagtcgatgctattggtggagctcgttttgatgatggaatgggaggggataatgaagtacaacggacaatgctggaattgatcaatcagttggatggatttgattcgcgcggtgcaattaaagttttaatggccacaaatcgtcccgatactttggatcccgcacttgtacggccaggacgtattgatcgtcgtattgaattcgccgtgcctgatctggaagcacgtgcaaatattctaaagattcatacaaaacggatgagtgttgatcggagtattcgttacgaattgatatcgcgtctttgcccgaatacaacaggtgctgatattcgctctgtctgtacagaagccggaatgtttgcattacgtgccccccgtaaagtgataactgaaaaagattttattgacgctgttcagaaagtggtgaaaggatatgcaaagtttagttcaactccaagttatatgacacataattaaaatcaatttctttgaatttgggcttttgataaatgtttcttaaataaaaaaa

SEQ ID NO: 52 - protein, 414 aa, Meloidogyne hapla
KEVKALGEEEIAALKSYNLGPYSEKLRQVETDIQEALKNINTLCGVKESDTGLAPPALWDLVADKTAIAQEQPLQVARCTKIIKTEGQDPRYMINVKQFAKFVVDLASAVAPTDIEEGMRVGVDRNKYQIHIPLPAKIDPSVTMMTVEEKPDVTYADVGGCKEQIEKLREVVEMPLLHPERFVNIGIEPPKGVLFYGPPGTGKTLCARAVANRTDAWFIRVIGSELVQKYVGEGARMVRELFEMAKTKKACIIFFDEVDAIGGARFDDGMGGDNEVQRTMLELINQLDGFDSRGAIKVLMATNRPDTLDPALVRPGRIDRRIEFAVPDLEARANILKIHTKRMSVDRSIRYELISRLCPNTTGADIRSVCTEAGMFALRAPRKVITEKDFIDAVQKVVKGYAKFSSTPSYMTHN

SEQ ID NO: 53 - DNA, 1308 bp, Caenorhabditis elegans
atgccagatcaccttggagatgacatgcgcaaaactaaaaaagatgataccaaggaggaagagaagaatttccaggctctcgacgaaggagacattgctgttttgaaaagatacgggcaaggtccatacgcggaacagctcaaaacattggacgcggatattgaaaactgtctgaagaaggtgaacgaactttctggagtgaaagaatcagacaccgggttggctccaccagctctatgggatattgctgctgataaacaagcaatgcagcaagaacagcctctccaagttgccagatgcacaaaaattatcaccagcgacaagcacgatccgcgttacttgatcaacgtaaaacagttcgctaagtttgttgtagaccttgctgattccgttgcacctactgatattgaggagggcatgagagtaggagtagaccgtaacaagtatcaaattcatcttccgttgcccgccaaaattgatccaaccgtcacaatgatgcaggttgaagaaaaaccagatgtaacatattcggatgtcggaggctgcaaggatcaaattgaaaagcttcgagaagttgtcgagactccacttcttcatcccgagcgttacgttaatctcggaatcgagccaccaaaaggagttttgctctacggtccaccaggaacaggaaagacgctttgcgctcgtgccgttgccaatcgaacagatgcttgcttcattcgtgttattggatcagagttggttcagaaatatgtcggagaaggagctcgaatggttcgtgagttgttcgaaatggctcgtaccaagaaggcatgtcttatcttctttgatgaaattgatgctgttggaggtgctcgttttgatgatggacaaggaggtgacaatgaagttcaacgtactatgctcgagttgattaaccaacttgacggattcgatccacgtggaaacatcaaggtgcttatggcaacaaacagaccggacactctcgatcccgctctcatgagacctggtcgattggatcgtaaagtcgaattcgctcttccagaccttgcaggtcgtgctcacattctcaagattcatgcaaaacaaatgagcgttgaaagagatattcgttatgatttacttgctcgtctgtgcccaaacagtacaggagccgaaattcgctcagtctgcaccgaagctggaatgtttgcaattcgtgctagaagaaaggtggcaactgaaaaagatttccttgaagctatcaataaggttgtcaagggatatgccaaattcagcgccactccaagatatctgacacataattaa

SEQ ID NO: 54 - protein, 435 aa, Caenorhabditis elegans
MPDHLGDDMRKTKKDDTKEEEKNFQALDEGDIAVLKRYGQGPYAEQLKTLDADIENCLKKVNELSGVKESDTGLAPPALWDIAADKQAMQQEQPLQVARCTKIITSDKHDPRYLINVKQFAKFVVDLADSVAPTDIEEGMRVGVDRNKYQIHLPLPAKIDPTVTMMQVEEKPDVTYSDVGGCKDQIEKLREVVETPLLHPERYVNLGIEPPKGVLLYGPPGTGKTLCARAVANRTDACFIRVIGSELVQKYVGEGARMVRELFEMARTKKACLIFFDEIDAVGGARFDDGQGGDNEVQRTMLELINQLDGFDPRGNIKVLMATNRPDTLDPALMRPGRLDRKVEFALPDLAGRAHILKIHAKQMSVERDIRYDLLARLCPNSTGAEIRSVCTEAGMFAIRARRKVATEKDFLEAINKVVKGYAKFSATPRYLTHN

SEQ ID NO: 55 - DNA, 1308 bp, Caenorhabditis briggsae
atgccagatcaccttggagatgatatgcgtaaaacaaagaagggcgagacaaccgaagaagagaaaaacttccaagcactcgacgaaggagatattgctgttttgaagagatacggtcaaggtccatatgccgagcagctgaaacagttggatactgacattgaaaactgtttgaagaaggtcaatgagctatccggcgtgaaagaatcggatacaggattggcaccgccagcgttatgggatattgctgctgacaagcaagctatgcagcaagaacaacctcttcaggtcgccagatgcacgaaaattatcaccagtgacaagcatgatccaagatatttgatcaatgtgaaacagttcgcaaaattcgtcgttgatcttgctgattccgttgcgccaactgatattgaggaaggaatgagagtcggagtcgatcgcaacaagtatcaaattcatcttccacttccagcaaaaattgatccaaccgtaacaatgatgcaagttgaagagaagccagatgtgacatactctgacgttggaggttgcaaggatcaaattgaaaaacttcgtgaagtagtcgaaactccacttcttcatccagagcgctacgtgaatctcggaattgaaccaccaaaaggagtgctgctctacggtccccctggaactggtaaaactctttgcgctcgtgctgttgccaatcgtaccgatgcttgcttcattcgagttatcggatccgaactcgttcaaaagtacgtcggagaaggagctcgtatggttcgtgagcttttcgaaatggctcgcaccaaaaaggcgtgtcttatcttcttcgatgaaatcgacgctgttggaggtgctcgctttgacgacggtcagggaggagacaacgaagttcaacgtactatgcttgaattgattaaccagcttgatggtttcgacccaagaggaaacattaaggtactgatggcaacaaacagaccagatacacttgaccctgccctcatgagaccaggtcgcctggatcgtaaagtcgaattcgctcttcctgatcttgtgggacgtgctcacattctcaagattcatgctaagcagatgagtgtggaaagagacattcgctacgacttgcttgctcgtctttgccctaacagtactggagctgaaattcgttcagtgtgcaccgaagctggaatgttcgctattcgtgccagaagaaaggttgccaccgaaaaggatttcctcgaagctatcaataaagttgtgaagggatatgccaaattcagtgcaacaccgagatacctcacacataactga

SEQ ID NO: 56 - protein, 435 aa, Caenorhabditis briggsae
MPDHLGDDMRKTKKGETTEEEKNFQALDEGDIAVLKRYGQGPYAEQLKQLDTDIENCLKKVNELSGVKESDTGLAPPALWDIAADKQAMQQEQPLQVARCTKIITSDKHDPRYLINVKQFAKFVVDLADSVAPTDIEEGMRVGVDRNKYQIHLPLPAKIDPTVTMMQVEEKPDVTYSDVGGCKDQIEKLREVVETPLLHPERYVNLGIEPPKGVLLYGPPGTGKTLCARAVANRTDACFIRVIGSELVQKYVGEGARMVRELFEMARTKKACLIFFDEIDAVGGARFDDGQGGDNEVQRTMLELINQLDGFDPRGNIKVLMATNRPDTLDPALMRPGRLDRKVEFALPDLVGRAHILKIHAKQMSVERDIRYDLLARLCPNSTGAEIRSVCTEAGMFAIRARRKVATEKDFLEAINKVVKGYAKFSATPRYLTHN

SEQ ID NO: 57 - DNA, 1652 bp, Heterodera glycines
ggtttaattacccaagtttgagtgaagataaaagtaattaatggacccttttcaccggaacgaggctaaagttgaggtcgccgttgagccaccaacggcgatagaccaacgggtggacgacataatgcaaacgggcagatttggagacggccgattggtgaagatggaagtggactatagcgctcaagtggacagtcagttgatagtggcagacaatttggccaaggaggggaagactgccgaggcaattgagtccttggaaaagctggaaaaggacagtcgcataaattgcgacatgcgttccaaccagcgcctgttgtgccacatggtcaaattggcatttgacgcgaataattggcaattgctctgcgaaactgcgaagacattgtgcaagaagcgtctgctgatcaagtcgagcatcaagaaaatggtccaagaatgctgcgaaatggtgccaaaagcgccagacgcgtcgtccaaatcgacgctcatcgacacactccgcgcagtgactgcgggaaagatttacctagaggtcgaaagagcgcggctgaccaaacaagtggccgaaaagttggaagccgagggaaaattggacgaagcgcgcgaaatgatgatggaactgcaagtggagacgtacggcacgatggaagtggaggaaaaggtcaattatttgctgcatcaaatgcgcctttccattgccaataatcattttacgcgtgcttcaattatttcacgtaaaatcagcacaaaatttttcgaacgcgaaggcactcaagtgcaattgatgaaattggaattttacaaatatatggtgcaaatcggactgagcgaaaacaactatttggatgtgtgcaaacactttctggcaattcttaacactccgcagatccaagaaaacaacgtcaagaaaattgagattctcaagtgtgtcgtgttttacttgctgctttcggctcatgacaacgaaaaatgggaacttttgcatcgagtgaatgcgatgagagaattggaacaaatacccaaacacaaagaactgctggaactgttcatccaccaggaattgatcttttggagcaaaaccattgagtccgaattcgccccaattttattcgctgctcaaccgccggtcgaagtcatttcggattcgtttctcccgtccacccacgtgtttccgatgaccaaagaggatggtcaaaagcgcagagaacgccttcatgactgtgtgggggaacataatgtgcgaatggtggccaaatattactcgcggatcactttccaacgaatggccaaacttctcgaattcggaattgagcaaatggaagcctttgtgtgcaaaatgattgtcgacggagtaatccccgaagtgaaaattcaccgcccttcgcaaattatttatttgagcccgaaaaagaacggcgcagaagtgctggacgaatgggtttttaacgttcgcaaattgaccgacacaatgaacaaagtcagtcagctgatcgcaaaggaggaaatggttcacggattgcaaatttctcagcggatttgaccgatgatatcgatacaaagaatcaattgattgttttattattgttttcccaaaaaaataaacgaattaaaaaaaaaaaaaaaaaaaaaaaaaaaaaaaaaaaaaaaaa

SEQ ID NO: 58 - protein, 500 aa, Heterodera glycines
MDPFHRNEAKVEVAVEPPTAIDQRVDDIMQTGRFGDGRLVKMEVDYSAQVDSQLIVADNLAKEGKTAEAIESLEKLEKDSRINCDMRSNQRLLCHMVKLAFDANNWQLLCETAKTLCKKRLLIKSSIKKMVQECCEMVPKAPDASSKSTLIDTLRAVTAGKIYLEVERARLTKQVAEKLEAEGKLDEAREMMMELQVETYGTMEVEEKVNYLLHQMRLSIANNHFTRASIISRKISTKFFEREGTQVQLMKLEFYKYMVQIGLSENNYLDVCKHFLAILNTPQIQENNVKKIEILKCVVFYLLLSAHDNEKWELLHRVNAMRELEQIPKHKELLELFIHQELIFWSKTIESEFAPILFAAQPPVEVISDSFLPSTHVFPMTKEDGQKRRERLHDCVGEHNVRMVAKYYSRITFQRMAKLLEFGIEQMEAFVCKMIVDGVIPEVKIHRPSQIIYLSPKKNGAEVLDEWVFNVRKLTDTMNKVSQLIAKEEMVHGLQISQRI

SEQ ID NO: 59 - DNA, 537 bp, Heterodera glycines
tcggactgagcgaaaacaactatttggatgtgtgcaaacactttctggcaattcttaacactccgcagatccaagaaaacaacgtcaagaaaattgagattctcaagtgtgtcgtgttttacttgctgctttcggctcatgacaacgaaaaatgggaacttttgcatcgagtgaatgcgatgagagaattggaacaaatacccaaacacaaagaactgctggaactgttcatccaccaggaattgatcttttggagcaaaaccattgagtccgaattcgccccaattttattcgctgctcaaccgccggtcgaagtcatttcggattcgtttctcccgtccacccacgtgtttccgatgaccaaagaggatggtcaaaagcgcagagaacgccttcatgactgtgtgggggaacataatgtgcgaatggtggccaaatattactcgcggatcactttccaacgaatggccaaacttctcgaattcggaattgagcaaatggaagcctttgtgtgcaaaatgattgtcgacggagtaa

SEQ ID NO: 60 - protein, 178 aa, Heterodera glycines
GLSENNYLDVCKHFLAILNTPQIQENNVKKIEILKCVVFYLLLSAHDNEKWELLHRVNAMRELEQIPKHKELLELFIHQELIFWSKTIESEFAPILFAAQPPVEVISDSFLPSTHVFPMTKEDGQKRRERLHDCVGEHNVRMVAKYYSRITFQRMAKLLEFGIEQMEAFVCKMIVDGV

SEQ ID NO: 61 - DNA, 790 bp, Heterodera glycines
aaaatccgtagccgagggaaaattggacgaagcgcgcgaaatgatgcttgaactgcaagtggagacgtacggcacgatggaagtggaggaaaaggtcaattatttgctgcatcaaatgcgcctttccattgccaataatcattttacgcgtgcttcaattatttcacgtaaaatcagcacaaaatttttcgaacgcgaaggcactcaagtgcaattgatgaaattggaattttacaaatatatggtgcaaatcggactgagcgaaaacaactatttggatgtgtgcaaacactttctggcaattcttaacactccgcagatccaagaaaacaacgtcaagaaaattgagattctcaagtgtgtcgtgttttacttgctgctttcggctcatgacaacgaaaaatgggaacttttgcatcgagtgaatgcgatgagagaattggaacaaatacccaaacacaaagaactgctggaactgttcatccaccaggaattgatcttttggagcaaaaccattgagtccgaattcgccccaattttattcgctgctcaaccgccggtcgaagtcatttcggattcgtttctcccgtccacccacgtgtttccgatgaccaaagaggatggtcaaaagcgcagagaacgccttcatgactgtgtgggggaacataatgtgcgaatggtggccaaatattactcgcggatcactttccaacgaatggccaaacttctcgaattcggaattgagcaaatgggagcctttgtgtgcaaaatgattgtcgacggagtaatc

SEQ ID NO: 62 - protein, 263 aa, Heterodera glycines
KSVAEGKLDEAREMMLELQVETYGTMEVEEKVNYLLHQMRLSIANNHFTRASIISRKISTKFFEREGTQVQLMKLEFYKYMVQIGLSENNYLDVCKHFLAILNTPQIQENNVKKIEILKCVVFYLLLSAHDNEKWELLHRVNAMRELEQIPKHKELLELFIHQELIFWSKTIESEFAPILFAAQPPVEVISDSFLPSTHVFPMTKEDGQKRRERLHDCVGEHNVRMVAKYYSRITFQRMAKLLEFGIEQMGAFVCKMIVDGVI

SEQ ID NO: 63 - DNA, 672 bp, Globodera rostochiensis
gatgcgcgaattggaacatttgccgacgcataaagagctgctggagctgttcatccaccaggaactgattttctggagtaaagccattgagtccaaatatgcaccaattttgtttggagctgatccgcctcccgaacttgtccgggacacgttgctgccgtctactcacgtgttcccgatgacccaagcggatggtcgaaggcgtagagaacgtcttcacgactgtgtgggcgaacataatgtgaggatggtgtcgaagtactactcgcggattacgtttcaacgtatggccaaactgctcgaatttggcgttgagcaaatggaaacttttgtgtgcaaaatgattgtcgacggagttattcctgaggcgaaaattcaccgcccctcgcaaataatttatttgagtccgaagaagaacagtgtggaagtgctagacgcgtgggtttttaatgttcgcaaattgaccgacacaatgaacaaagtgagtcagctgactgcaaaggaggaaatggtgcccggattgcaaattgcgcagcgagtttgacgatcagcagaagttgaacgaatgttttgcaacttttgttattaaatacattttattgttttgcaggagatcaatgaggttttaaaaaaaaaaaaaaaaccccgggtgaggggacatgtcggccgcct

SEQ ID NO: 64 - protein, 180 aa, Globodera rostochiensis
MRELEHLPTHKELLELFIHQELIFWSKAIESKYAPILFGADPPPELVRDTLLPSTHVFPMTQADGRRRRERLHDCVGEHNVRMVSKYYSRITFQRMAKLLEFGVEQMETFVCKMIVDGVIPEAKIHRPSQIIYLSPKKNSVEVLDAWVFNVRKLTDTMNKVSQLTAKEEMVPGLQIAQRV

SEQ ID NO: 65 - DNA, 1473 bp, Caenorhabditis elegans
atggccgacagacgcgaaccgatcccggttgacccggtcgatgtcggagaactaagcgaacttgacaatttggctcatttggccgctcatggtggagatggtcgtttgttcaaaatggagcaggactactcgaaacaagtcgacgaagcacttctgaaagcccgagatatcgctcaaaaggacgctgttgctgctgtagagagtctgaacaacatcgaaaagctcactcgtcttggagctgacatgaagagcaatacacgcgttgttcaatatatgacgaagctttgtttcgaagggcagaaatgggatcttcttatggaaacaatcatgacgctttcaaagaaaagacttctcatcaaaatggctatcgcaaagatggtccgtgatgctgtcgcaatgattgacaaaatgccaaccgaggacctcaagatgaagcttattgaaactcttcgcactgttacagctggaaagatctatgtggaagtggaacgtgctcgcctcacctcgatggttgtgaagaagttggagagagagggaaagcttgacgaagctgcaaccatgcttcttgaacttcaagtggaaacatatggctccatggaaatgcgagagaaggtgcaataccttctcgagcaaatgagatattctctcgttcgtaatgattttgtgcgagccactattatctctaagaaaatcaatatcaagttcttcaacaagtcagacgaagatgaagtacagaacctgaagctgaaatactacgattcgatgatccgtatcggacttcacgatggaaactatctggacgtgtgccgtcatcatcgtgaaatttatgagacgaaaaagattaaagctgattcagccaaggcgacctcacatcttcgttctgccattgtctactgtcttttggctccgcacacaaacgagcaatgggatctcctcaaccgaattgccattcaaagagaattggaaaccgttccagactacaagatcattctggatcttttcatcaaccaagaactcatttctttcaaaggaaccattgtcgcaaagtacgagaagcttttaagacgtggaacaactagctcaccagacaccggaattttcgacaaatcaactgaaggagagaagcggtggtctgatttgcaccttcgtgttggagagcacaacatgcgtatgattgccaagtactatactcaaatcactttcgaacgtctcgctgagctcctcgacttcccagttgatgaaatggaatcatttgtgtgtaacctcattgttaccggtcagatcaccggagcaaaacttcatcgcccttcccgtattgtcaatcttcgtttgaaaaaggcaaatgttgagcaattggatgtatgggcgagcaacgtgcacaaactgacggatacattgaacaaagtttcccatctcattctgaaagagcaaatggttcacaagaacttggatctcgcggccccacgggcataa

SEQ ID NO: 66 - protein, 490 aa, Caenorhabditis elegans
MADRREPIPVDPVDVGELSELDNLAHLAAHGGDGRLFKMEQDYSKQVDEALLKARDIAQKDAVAAVESLNNIEKLTRLGADMKSNTRVVQYMTKLCFEGQKWDLLMETIMTLSKKRLLIKMAIAKMVRDAVAMIDKMPTEDLKMKLIETLRTVTAGKIYVEVERARLTSMVVKKLEREGKLDEAATMLLELQVETYGSMEMREKVQYLLEQMRYSLVRNDFVRATIISKKINIKFFNKSDEDEVQNLKLKYYDSMIRIGLHDGNYLDVCRHHREIYETKKIKADSAKATSHLRSAIVYCLLAPHTNEQWDLLNRIAIQRELETVPDYKIILDLFINQELISFKGTIVAKYEKLLRRGTTSSPDTGIFDKSTEGEKRWSDLHLRVGEHNMRMIAKYYTQITFERLAELLDFPVDEMESFVCNLIVTGQITGAKLHRPSRIVNLRLKKANVEQLDVWASNVHKLTDTLNKVSHLILKEQMVHKNLDLAAPRA

SEQ ID NO: 67 - DNA, 1476 bp, Caenorhabditis briggsae
atggccgataagcgtgagccaattccggttgatcctgtcgacatcggcgagaccaccgatctcgacagtttggctcatttggcggctcatggcggagatggtcgtcttttcaaaatggagcaagattacacaaaacaagtagacgaagcgctcctcaaagctcgtgatctggctcaaaaagatgttctcgctgctgtagagagtcttaacaatattgagaagctcactcgtcttggtgcagatatgaagagcaatactcgtgttgttcagtacatgaccaaactttgcttcgaaggtcaaaaatgggatcttctgatggaaactatcatgacactctccaagaagagacttctcatcaagatggccatcgccaaaatggttcgtgacgcagtcgccatgatagataagatgccaagcgacgatctcaaaatgaaactcatcgaaacccttcgcactgtcaccgctggcaaaatctatgttgaagttgagcgtgctcgtcttacatctatggttgtcaagaagttggaagctgagggaaagcttgatgaggccgctacgatgcttctcgagcttcaagtcgagacatatggttctatggagatgaaagagaaggtttcctaccttttggagcaaatgagacactcgttggttcgcaacgactacgttcgtgcaacaatcatttccaagaagattaacattaaatttttcaacaaatctgacgctgaggatgtgcaagatttgaagttgaagtactatgatttgatgatccgtattggactccacgacggaaactatttggatgtctgtcgccaccatcgtgaaatttacgaaaccataaagatcagagcggatccggcaaaagcggcatcgcaccttcgctcggctatcgtctactgtcttctggcacctcacaacaatgagcaatgggatctgttgaatagaattgcgattcagagagaattggaaacagttccggactacaaaatcattctcgatttgtttatcaatcaggaattgatttctttcaagggaaccattgtggctcagtacgagaagttgttgagacgtggaacaccgacgtctccagacactggaatcttcgacaagtcgcaggaaggagaaaagcggtggtcggatttgcaccttcgtgttggagagcataacatgcgtatgatcgccaaatactacacccaaatcacattcgaacgccttgccgagcttctagacttcccagttgacgagatggaatccttcgtttgcaatctcattgtcagcggccagatcatcggcgccaaacttcatcggccatctcgtatcgtgaatctgcgtttgaagaaggcaaatgtggagcagttggatgtttgggcgagcaatgtgcacaaactgacggatacgttgaacaaagtgtcccatttgattctcaaggagcagatggttcataagaatttggatctctcggcggcgccacgtgcatga

SEQ ID NO: 68 - protein, 491 aa, Caenorhabditis briggsae
MADKREPIPVDPVDIGETTDLDSLAHLAAHGGDGRLFKMEQDYTKQVDEALLKARDLAQKDVLAAVESLNNIEKLTRLGADMKSNTRVVQYMTKLCFEGQKWDLLMETIMTLSKKRLLIKMAIAKMVRDAVAMIDKMPSDDLKMKLIETLRTVTAGKIYVEVERARLTSMVVKKLEAEGKLDEAATMLLELQVETYGSMEMKEKVSYLLEQMRHSLVRNDYVRATIISKKINIKFFNKSDAEDVQDLKLKYYDLMIRIGLHDGNYLDVCRHHREIYETIKIRADPAKAASHLRSAIVYCLLAPHNNEQWDLLNRIAIQRELETVPDYKIILDLFINQELISFKGTIVAQYEKLLRRGTPTSPDTGIFDKSQEGEKRWSDLHLRVGEHNMRMIAKYYTQITFERLAELLDFPVDEMESFVCNLIVSGQIIGAKLHRPSRIVNLRLKKANVEQLDVWASNVHKLTDTLNKVSHLILKEQMVHKNLDLSAAPRA

SEQ ID NO: 69 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta

SEQ ID NO: 70 - DNA, 609 bp, Glycine max
gaagccacgtcatgaagagtatatcatttcagtaatgttttgagacgcctctataatgctttaccaacaaaacaaaacaaaaaaaagaacatttgaaaccatttgtattaaaaaaaaaaaggtatattaggccataatattataggtaacatgaaatatcaaatgacacgcaagagttttgtcaaaaatgaaaccatcacacatcagagattatggcaaataatgttttgtgtgtctcttgcttcacccataacataagcctctataactggagagaagaaaaaaaaaagtggaggggctagggtgggaatttggaagaatacagttatattgagcattgagcaagttgatagaaagcttctcaatttgtacaaaatttgcatccacatgattattaaagacgtagacagcacttcttccttctttttttctataagtttcttatatattgttcttcatgttttaatattattactttatgtacgcgtctaacagtagtcctcccaaactgctataaatagagcctcttcaacgcacctcttggcagtacaaaaattattcatctcttctaagttctaattttctaagcattcagtaaaagaactaacc

SEQ ID NO: 71 - DNA, 1476 bp, Arabidopsis thaliana
gctcgcgttagttccactcaaggagtatcctttcttccttgcgcaactctccaccttcgggtaaagtaccatctctagcatcttgagtcttgatcaacttctgttttgcttactctcaaaatgcattaatttttttttatactagatcatagtattatatctcttaatctacctattgaaatctacttaatgtttttactaaaacctacgtgtttctctttagagaattttgtgctatgcatgaattagaggttagtaatgtgtaatacttcataagtctagatttatttgttggttaacacgtttagtaattcacacacacacaccaccttagatattttactgtgaattagaaaaagatacatagttaggagtgtttttttaaaaaaattcaatcatgagaaaattagaggtgtgatgttatacattatgaaaatgcaaagggcagatacgaataaattagaaacttgtttaacgggtcagagttggcttctagtctctttcgacttggatacttcttcttctacaattgggacattattgtaggcgcattatatcatttctctacatgcaatgaatgtacatacattaattcacatttatttttggaataatcatatgagtgatcgaagtttgtatttatatattcaatcttcacaaactacttttatttaaaaatcatttgcaaaatgctattttattgacaaaaagatatatgctataaaataaaataaaattcacaaactatagtcattaatacaaaaagaaatcattgaatatggtagaggggaaacaaaaaaaaaacacgacgatgtaagttggtggaaccacattatcaaaataaaagaaggtggtggaaccaaattgaataaagtccgtccatatcattatccgtcccttaggagcctctaattagtaatattcttatgggtccactgtggcttagaggacttgattaaaaccattcttatttagtgctaactttgtgagggttggaataacgaaccaagctgattcaaaccattccaaaacaaagttgtcacatatttcaaaaccaaagtttaccggacagagaaatatggtgtgtttttctcaaaccaagctaaatggaatccattgtaaaccaaaatgttcacacctacctattcttttggagtcccttttccatgtgtttgctgtctgctagtcaagtttcattagctgattgccttgcatcatattcttggatcaactttttttttttttttttttggggtaattaacaaaatgcttaaatttctcaagactataggatcacattacctgtgtgcttaacataacttttagataggctagagaattgatctattacaagataatcaataatttacagaagaaaacattcttttttttgttctatttccttcatgtaggtatgtagctgtatattatactatcttgtattttcgatatcgtgctggaactgtcacagatgca

SEQ ID NO: 72 - DNA, 23 bp, Unknown
  motif
atgacncgngggtcaagcgccgg

SEQ ID NO: 73 - DNA, 21 bp, Unknown
  motif
cacatcacnattttctcaccn

SEQ ID NO: 74 - DNA, 24 bp, Unknown
  motif
caagttgaatangcgttcaaagct

SEQ ID NO: 75 - DNA, 23 bp, Unknown
  motif
gatganactgccgtaattgccgt

SEQ ID NO: 76 - DNA, 38 bp, Unknown
  motif
ttgtcntcaacngtcagttgcggtgttatcggcattgt

SEQ ID NO: 77 - DNA, 26 bp, Unknown
  motif
tatgagaatggntatgaaatgccaat

SEQ ID NO: 78 - DNA, 26 bp, Unknown
  motif
aaaatggccgagatcaaccagtacta

SEQ ID NO: 79 - DNA, 27 bp, Unknown
  motif
ctgctgganctgttcatccaccaggaa

SEQ ID NO: 80 - DNA, 24 bp, Unknown
  motif
gactgtgtgggngaacataatgtg

SEQ ID NO: 81 - DNA, 26 bp, Unknown
  motif
caacgnatggccaaactnctcgaatt

SEQ ID NO: 82 - DNA, 46 bp, Unknown
  motif
ttgagcaaatggaancntttgtgtgcaaaatgattgtcgacggagt

SEQ ID NO: 83 - DNA, 36 bp, Unknown
  motif
gaaaattcaccgcccntcgcaaatnatttatttgag

SEQ ID NO: 84 - DNA, 44 bp, Unknown
  motif
tgggtttttaangttcgcaaattgaccgacacaatgaacaaagt

SEQ ID NO: 85 - DNA, 33 bp, Unknown
  motif
gcaaaggaggaaatggtncncggattgcaaatt

SEQ ID NO: 86 - DNA, 21 bp, Unknown
  motif
aatgtgttcatcaatcgtcag

SEQ ID NO: 87 - DNA, 26 bp, Unknown
  motif
ggtggcgaaattgcttcaacatttga

SEQ ID NO: 88 - DNA, 35 bp, Unknown
  motif
ttggcgaagacactttgctccgcttttccggtgtc

SEQ ID NO: 89 - DNA, 22 bp, Unknown
  motif
tgcacgacgcgctttgtgtgct

SEQ ID NO: 90 - DNA, 21 bp, Unknown
  motif
tcaaaaggtggccggnaagga

SEQ ID NO: 91 - DNA, 25 bp, Unknown
  motif
aattgccnacaatcatttgcgacaa

SEQ ID NO: 92 - DNA, 23 bp, Unknown
  motif
gtnattgacgatcaacacgcaat

SEQ ID NO: 93 - DNA, 22 bp, Unknown
  motif
tntcatttgtggacaangaaca

SEQ ID NO: 94 - DNA, 44 bp, Unknown
  motif
ccgaagggcgtnntgttntttggtccgccgggcaccggcaaaac

SEQ ID NO: 95 - DNA, 56 bp, Unknown
  motif
gcccgcgcggtngccaatcggacggacgcntgtttcatncgcgtcatcggttccga

SEQ ID NO: 96 - DNA, 23 bp, Unknown
  motif
ggcgaaggcgcncgcatggtgcg

SEQ ID NO: 97 - DNA, 56 bp, Unknown
  motif
aanacgaaaaaggcgtgcattctcttcttcgacgangtngacgccatcggcggngc

SEQ ID NO: 98 - DNA, 56 bp, Unknown
  motif
cgcggggccatnaaggttttgatggccaccaacagaccggacacnctcgacccggc

SEQ ID NO: 99 - DNA, 26 bp, Unknown
  motif
caaattcacaccaaacggatgagcgt

SEQ ID NO: 100 - DNA, 23 bp, Unknown
  motif
aacattcgntacgaattgattgc

SEQ ID NO: 101 - DNA, 34 bp, Unknown
  motif
ttgcgnagcgtttgcactgaggcgggaatgttcg

SEQ ID NO: 102 - DNA, 26 bp, Unknown
  motif
atnacggagcaagacttnctcaaggc

SEQ ID NO: 103 - DNA, 26 bp, Unknown
  motif
tcaacnccggcntatatgacgcacaa

SEQ ID NO: 104 - DNA, 1425 bp, Heterodera glycines
ggtttaattacccaagtttgagatatttattaattatcatcatttttggttttacaaatgggtcaaaaccaaccgcagggaggtgggcgaaagccaggcgatgacaaaggacaggaccctgaaaaaaaacggaaataccatggaccgccggttccaacgcgaattggaaaacgcaagaagggctctcgtggtcccgacacagcaaacaaaatgcccaccgtgactccgatcactcgttgtaaactcaagctcctcaagtatgaccggattaaggactatcttttaatggaggaagaattcataaagaacatggagcgtttgaagcctcaggacgaacgtcaggaggaagagcgtgttaaagttgacgaccttcgtgggactccaatgtctgtcggatcattggaagaagtcattgacgatcaacacgcaattgtttccacgaatgtcggcagtgaacattacgtcaacattttgtcatttgtggacaaagaacagcttgagccgggctgtgccgttctgctgaatcacaaaacccactcaatcgtcggcgttcttgcggaggacgccgatccgatggtgtcggtgatgaaattggagaaagcaccgaccgagacttacgctgacgttggtggcctcgagcagcaaattcaggagatcaaagaggcggtggagttgccgttgacacacccggaatattacgaggaaatgggcatcaaaccgcccaaaggcgtcattctctacgggtcgcccggcactggcaaaacgctgcttgcaaaggccgttgccaaccaaacttctgccacctttttgcgcgtcgttggctccgaactcatccaaaagtatttgggcgacgggccgaaaatggtccgcgaattgtttcgcgttgcagaagaacatgcgccgtcaatcgttttcatcgacgaaatcgacgccattggaaccaaacggtacgattcgaattccggcggagagcgcgagattcagcgcacgatgctggaactgctcaatcagttggacggctttgactcgcgtggcgacgtcaaagtgctgatggcgaccaatcggatcgactcgttggacccggcacttatccgtcccggacgaatcgaccgcaaaatcgaattcccacttccggacgaaaagaccaagcggaggattttccacattcacactgctcggatgcaactggaaaatgtggacttggaggaatttatagcagccaaagacgatctgtcgggagccgacataaaggcaatgtgcactgaggcgggtttgcttgcactgcgcgaacggcgaatgaaagtgacaatggacgacatgcgaaaggcgaaggaaaatgttctttaccggaagaaagacaacgcacccgaaacgatgtatctttgaagggttcccaaaattggaatttgtgttgacttttattca

SEQ ID NO: 105 - protein, 442 aa, Heterodera glycines
MGQNQPQGGGRKPGDDKGQDPEKKRKYHGPPVPTRIGKRKKGSRGPDTANKMPTVTPITRCKLKLLKYDRIKDYLLMEEEFIKNMERLKPQDERQEEERVKVDDLRGTPMSVGSLEEVIDDQHAIVSTNVGSEHYVNILSFVDKEQLEPGCAVLLNHKTHSIVGVLAEDADPMVSVMKLEKAPTETYADVGGLEQQIQEIKEAVELPLTHPEYYEEMGIKPPKGVILYGSPGTGKTLLAKAVANQTSATFLRVVGSELIQKYLGDGPKMVRELFRVAEEHAPSIVFIDEIDAIGTKRYDSNSGGEREIQRTMLELLNQLDGFDSRGDVKVLMATNRIDSLDPALIRPGRIDRKIEFPLPDEKTKRRIFHIHTARMQLENVDLEEFIAAKDDLSGADIKAMCTEAGLLALRERRMKVTMDDMRKAKENVLYRKKDNAPETMYL

SEQ ID NO: 106 - DNA, 23 bp, Unknown
  motif sequence
caaacntctgcnacctttttgcg

SEQ ID NO: 107 - DNA, 38 bp, Unknown
  motif sequence
gaagaacatgcnccgtcaatngttttcatcgacgaaat